(19) United States
(12) Patent Application Publication
(10) Pub. No.: US 2026/0356872 A1
(43) Pub. Date: Nov. 12, 2026

(54) Glucosamine Orotate Salt, Compositions Thereof, and Methods of Making and Use Thereof

(71) Applicant: Pharma Cinq, LLC, Rockville, MD (US)

(72) Inventors: Reid Von Borstel, Rockville, MD (US); David Simpson, Rockville, MD (US); Fedor Romantsev, rockville, MD (US); Rolando Alejandro Garcia Garcia, Rockville, MD (US); Surya P. Aryal, Rockville, MD (US)

(21) Appl. No.: 19/661,197

(22) Filed: Apr. 28, 2026

Related U.S. Application Data
(60) Provisional application No. 63/796,088, filed on Apr. 28, 2025.
(60) Provisional application No. 63/894,445, filed on Oct. 6, 2025.

(51) Int. Cl. C07H 5/06; A61K 31/198; A61K 31/7008; A61P 1/16; A61P 19/02; A61P 43/00; C07H 1/06

(52) U.S. Cl. CPC C07H 5/06; A61K 31/198; A61K 31/7008; A61P 1/16; A61P 19/02; A61P 43/00; C07H 1/06

(57) ABSTRACT

Glucosamine orotate salt and methods of production thereof have been developed. The glucosamine orotate salt allows co-delivery of two precursors, orotate for augmenting uridine nucleotides and glucosamine. Together, they achieve augmentation of intracellular UDP-sugars for glycosaminoglycans (GAGs) and hyaluronan (HA) synthesis. The glucosamine orotate salt shows surprisingly high water solubility (e.g., a water solubility of at least 1 g/mL, at a temperature from 25 °C to 27 °C and under a pressure of 1 atm), which is at least one order of magnitude greater than any previously reported orotate salts. The glucosamine orotate salt can be prepared by passing glucosamine hydrochloride through an ion exchange column and into a suspension of orotic acid. Compositions containing glucosamine orotate salt and methods of using thereof are also disclosed.

### CROSS-REFERENCE TO RELATED APPLICATIONS

This application claims the benefit of and priority to U.S. Provisional Application No. 63/796,088 filed Apr. 28, 2025, and U.S. Provisional Application No. 63/894,445 filed Oct. 6, 2025, which are herein incorporated by reference in their entireties.

### TECHNICAL FIELD

The present disclosure relates to glucosamine orotate salt, compositions thereof, and methods of making and use thereof.

### BACKGROUND

A number of clinical conditions are related to impaired synthesis of the classes of oligosaccharides known as glycosaminoglycans (GAGs) and hyaluronan (HA), also known as hyaluronic acid. The vascular endothelial glycocalyx contains GAGs and HA, which are covalently bound to anchor proteins, such as Syndecans and CD44, or attached by entanglement with other GAGs and HA. The endothelial glycocalyx is the lining on the interior surface of blood vessels including capillaries. The endothelial glycocalyx can prevent contact of blood cells, including pro-inflammatory neutrophils, with the endothelial surface. The glycocalyx is also important for vascular endothelial mechanosensing of laminar versus turbulent blood flow, which in turn regulates endothelial growth patterns. Impaired glycocalyx integrity predisposes blood vessels for formation of atherosclerotic plaques, resulting in impaired blood-brain barrier function, which contributes to neurological problems, including cognitive decline, “brain fog”, and dementia. Inflammatory stimuli originating in endothelial cells or other tissues can trigger glycocalyx shedding, contributing to clinical pathology in acute trauma, systemic inflammation, and infections, such as SARS-CoV2 (COVID) infections. For example, patients infected with SARS-CoV2 (COVID) who have a thin glycocalyx are more likely to develop an endothelial cell viral infection, which in turn further damages the glycocalyx, causing its shedding. This shedding of glycocalyx correlates with infection severity and poor clinical outcomes, including mortality.

Glycocalyx integrity represents a balance between degradation and resynthesis. Biosynthesis of GAGs and HA occurs at the cell membrane, where intracellular uridine-diphospho-sugars (UDP-sugars) provide the saccharide units that are joined by hyaluronic acid synthases and corresponding GAG synthases, and extrude into the extracellular space to produce or restore the glycocalyx. Similarly, synthesis of cartilage in joints, tendons, and other connective tissues including skin, requires formation of GAGs and HA as major components. Synthesis of GAGs and HA for cartilage also requires UDP-sugars as immediate precursors. Cartilage maintenance involves a balance between degradation (due to inflammation, wear, and age-related attrition of active cells that produce GAGs and HA in joints and tendons) and synthesis by viable chondrocytes and synoviocytes. Osteoarthritis is a common condition reflecting diminished capacity for cartilage maintenance and repair.

Glucosamine, an amino sugar, can be synthesized in cells or taken up into cells from the circulation and incorporated into intracellular amino sugar metabolite pools. UDP-glucosamine (UDP-GlcN and its downstream congener UDP-N-acetylglucosamine (UDP-GlcNac) can serve as GAG or HA precursors. Glucosamine, especially a salt thereof, glucosamine sulfate or glucosamine hydrochloride, is widely used to reduce joint pain due to osteoarthritis. However, the glucosamine sulfate salt alone provides only marginal therapeutic effects on GAG, HA or cartilage when administered at recommended dosages, delivering concentrations to synovial fluid below those required to augment GAG synthesis in vitro. The downstream metabolite N-acetylglucosamine that is one biochemical step closer to UDP-N-acetylglucosamine, the immediate precursor for HA synthesis, is poorly transported and incorporated into cells, and thus has limited potential activity.

Orotic acid is an intermediate in de novo synthesis of uridine nucleotides. It is converted into intracellular uridine monophosphate (UMP) by the enzyme complex UMP Synthase, which has two enzymatic activities. This conversion includes: (1) reaction between phosphoribosyl pyrophosphate (PRPP) and orotic acid catalyzed by orotate phosphoribosyl transferase (OPRTase) to yield orotidine 5′-monophosphate (OMP), and (2) conversion of OMP to UMP catalyzed by OMP Decarboxylase. UMP is then phosphorylated by nucleotide kinases to yield UDP and UTP. UTP undergoes enzymatic reaction with various intracellular sugars to produce UDP-sugars, the immediate precursors to both GAGs and HA. However, orotic acid and existing orotate salts have very poor water solubility, which can limit the rate and extent of dissolution after oral administration, thereby limiting delivery into the circulation. Further, orotic acid cannot be delivered in effective amounts via the intravenous route, again due to poor solubility.

There remains a need for compositions for effectively delivering glucosamine and orotate to mammals.

Therefore, it is the object of the present invention to provide a glucosamine orotate salt.

It is a further object of the present invention to provide compositions containing a glucosamine orotate salt.

It is a further object of the present invention to provide methods for making a glucosamine orotate salt.

It is a further object of the present invention to provide methods for supporting the health of blood vessels.

It is a further object of the present invention to provide methods for supporting synthesis and maintenance of cartilage in joints and tendons.

It is a further object of the present invention to provide methods for supporting the health of the liver.

### SUMMARY OF THE INVENTION

Glucosamine orotate salt (also referred to herein as “glucosamine orotate” or “GO”) and methods of production thereof have been developed. The glucosamine orotate salt allows co-delivery of two precursors, a uridine nucleotides precursor (i.e., orotate) and glucosamine, to achieve augmentation of intracellular UDP-sugars for GAG and HA synthesis. The two precursors are provided in a single salt, without the need for inorganic counter ions that may affect ion homeostasis. In some forms, the glucosamine orotate salt is in a tetrahydrate, dihydrate, and/or 0.8 hydrate crystal form that is stable (e.g., the percentage of weight change is less than 1% for at least 6 months) at room temperature (e.g., from 20 °C to 30 °C under a pressure of 1 atm, such as 25 °C to 27 °C under a pressure of 1 atm) in open air. The glucosamine orotate salt shows surprisingly high water solubility (e.g., a water solubility of at least 1 g/mL, such as about 1.56 g/mL, at a temperature from 25 °C to 27 °C and under a pressure of 1 atm), which is at least one order of magnitude greater than any previously reported orotate salts, such as arginine orotate, lysine orotate, potassium orotate, and meglumine orotate. The high water solubility (≥1 g/mL) of glucosamine orotate salt may result in improved bioavailability compared to free orotic acid (with a solubility of about 0.1 mg/mL in water) and poorly soluble orotate salts of metal ions such as potassium or magnesium, where slow dissolution after administration may limit the rate and extent of absorption. Further, surprisingly, the glucosamine orotate salt has an improved taste (neutral-sweet) compared to glucosamine, which has an unpleasant salty and bitter taste, and orotate, which has a sour taste, separately.

Methods for producing glucosamine orotate salt are disclosed. Previously reported methods for producing other organic or metal cation salts of orotic acid (arginine orotate, lysine orotate, potassium orotate, meglumine orotate, etc.) by simple mixing a suspension of orotic acid in water with a solution of a cationic compound or a metal ion, for example, using glucosamine hydrochloride, fails to produce glucosamine orotate salt (other than at most a negligible amount). For example, simple mixing of orotic acid in water with glucosamine hydrochloride fails to produce glucosamine orotate salt in an amount that can be separated from the mixing solution using known methods, such as crystallization. In some forms, the methods for producing glucosamine orotate salt involve combining glucosamine free base with orotic acid to form the glucosamine orotate salt. For example, the methods for producing glucosamine orotate salt include passing glucosamine hydrochloride through an ion exchange column, to form glucosamine free base, and into a suspension of orotic acid to form the glucosamine orotate salt. The molar ratio of the glucosamine hydrochloride to orotic acid is preferably about 1:1, and can range from 0.8:1 to 1.1:0.9, such as 0.8:1, 0.85:1, 0.9:1, 1:1.1, 1:1, 1:0.9, 1:0.85, 1.1:1, 1.2:1, 1.2:1.1, or 1.1:0.9. In some other forms, the methods for producing glucosamine orotate salt involve combining a silver salt of orotate with an aqueous solution of glucosamine hydrochloride to form the glucosamine orotate salt; silver hydrochloride has low water solubility and precipitates, leaving glucosamine orotate in solution. The silver chloride is removed by settling, centrifugation or filtration.

Compositions containing glucosamine orotate salt are provided herein. The compositions are pharmaceutical compositions, cosmetic compositions, or nutraceutical (dietary supplement compounds derived from foods with potential pharmacologic activity) compositions. Pharmaceutical, cosmetic, and nutraceutical compositions containing glucosamine orotate salt and optionally one or more suitable carrier(s) and/or excipient(s) are disclosed. In some forms, the composition (e.g., pharmaceutical composition or nutraceutical composition) is in a suitable form, such as a pill, capsule, tablet, dry powder, or liquid form, for oral administration to a subject. For example, the composition is in the form of a liquid (e.g., a ready-to-drink liquid), dry powder (e.g., a drink mix), a capsule, a tablet, a soft or hard chew, or a gummy, that is suitable for oral administration. In some forms, the improved solubility of glucosamine orotate salt allows for liquid solution formulations thereof for intravenous or subcutaneous injection or infusion. In some forms, the composition containing glucosamine orotate salt is in a suitable form for topical application to the skin. Compositions containing glucosamine orotate salt in combination with additional active ingredients are also disclosed.

In some forms, the glucosamine orotate salt in the composition is in an effective amount to restore, improve, or support the health of blood vessels; to improve maintenance or repair of cartilage in joints or tendons; and/or to restore, improve, or support the health of liver. In some forms, the glucosamine orotate salt in the composition is in an effective amount to promote synthesis of glycosaminoglycans (GAGs) and hyaluronan (HA) and/or reduce local and/or systemic inflammation, thereby restoring, improving, or supporting the health of blood vessels, improving maintenance or repair of cartilage in joints or tendons, and/or restoring, improving, or supporting the health of liver in a subject. In some forms, the glucosamine orotate salt in a topical composition is in an effective amount to support GAG and HA synthesis locally in the skin. In some forms, the glucosamine orotate salt in the composition is in an effective amount to improve or maintain the health of the endothelial glycocalyx of a subject. In some forms, the glucosamine orotate salt in the composition is in an effective amount to reduce and/or ameliorate one or more symptom(s) (e.g., exacerbation of a chronic inflammation) associated with a mitochondrial disease or disorder of a subject. In some forms, the glucosamine orotate salt in the composition is in an effective amount to reduce and/or ameliorate one or more symptom(s) (e.g., chronic inflammation) associated with a disease or disorder associated with cGAS-STING activation, such as trauma, infection, or a disease or disorder that is at least partially a pathophysiological consequence of cGAS-STING activation (e.g., cytokine storm).

Methods for restoring, improving, or supporting the health of blood vessels in a subject, methods for improving maintenance or repair of cartilage in joints or tendons in a subject, methods for supporting the health of the liver (such as by protecting the liver against inflammatory damage or reducing inflammation in the liver) in a subject, and methods for improving the appearance of a subject's skin (such as by reducing wrinkles), using glucosamine orotate salt or compositions containing glucosamine orotate salt are disclosed. Methods for improving or maintaining mitochondrial function of a subject, methods for reducing and/or ameliorating one or more symptom(s) (e.g., chronic inflammation) associated with a mitochondrial disease or disorder in a subject, and methods for reducing and/or ameliorating one or more symptom(s) (e.g., chronic inflammation) associated with a disease or disorder associated with cGAS-STING activation, such as trauma, infection, or a disease or disorder that is at least partially a pathophysiological consequence of cGAS-STING activation (e.g., cytokine storm) in a subject using glucosamine orotate salt or compositions containing glucosamine orotate salt are also disclosed.

### BRIEF DESCRIPTION OF THE DRAWINGS

FIGS. 1A-1B are graphs showing the PC2-D plasma orotic acid levels (FIG. 1A) and the plasma uridine+uracil levels (FIG. 1B) in mice administered with glucosamine orotate, magnesium orotate, and orotic acid.

FIGS. 2A-2B are graphs showing the plasma orotate levels (FIG. 2A) and the plasma uridine+uracil levels (FIG. 2B) in mice administered with different levels of glucosamine orotate tetrahydrate.

FIGS. 3A-3B are graphs showing the chromatographs of glucosamine orotate tetrahydrate (FIG. 3A) and orotic acid (FIG. 3B).

FIGS. 4A-4B are microscopic images of glucosamine orotate tetrahydrate crystals at 40× magnification (FIG. 4A) and 10× magnification (FIG. 4B).

FIGS. 5A-5D are bar graphs showing the serum levels of representative markers for inflammatory liver cell injury in GO-treated mice and vehicle-treated mice: Alanine Aminotransferase (FIG. 5A), Aspartate Aminotransferase (FIG. 5B), Total Bilirubin (FIG. 5C), and Direct Bilirubin (FIG. 5D).

### DETAILED DESCRIPTION OF THE INVENTION

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### I. Glucosamine Orotate Salt

Glucosamine orotate salt and methods for producing thereof are described herein. Typically, the molar ratio of glucosamine cation and orotate anion in the glucosamine orotate salt is about 1:1.

The glucosamine in the salt is in a racemic form or optionally in an isomer form. For example, preferably the glucosamine orotate salt is D-glucosamine orotate salt. In some forms, the glucosamine orotate salt is an isolated glucosamine orotate compound. For example, preferably the isolated glucosamine orotate salt is D-glucosamine orotate salt. In some forms, the glucosamine orotate salt has the following structure:

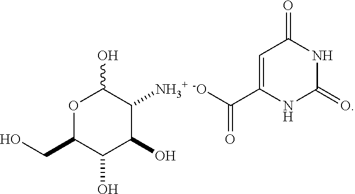

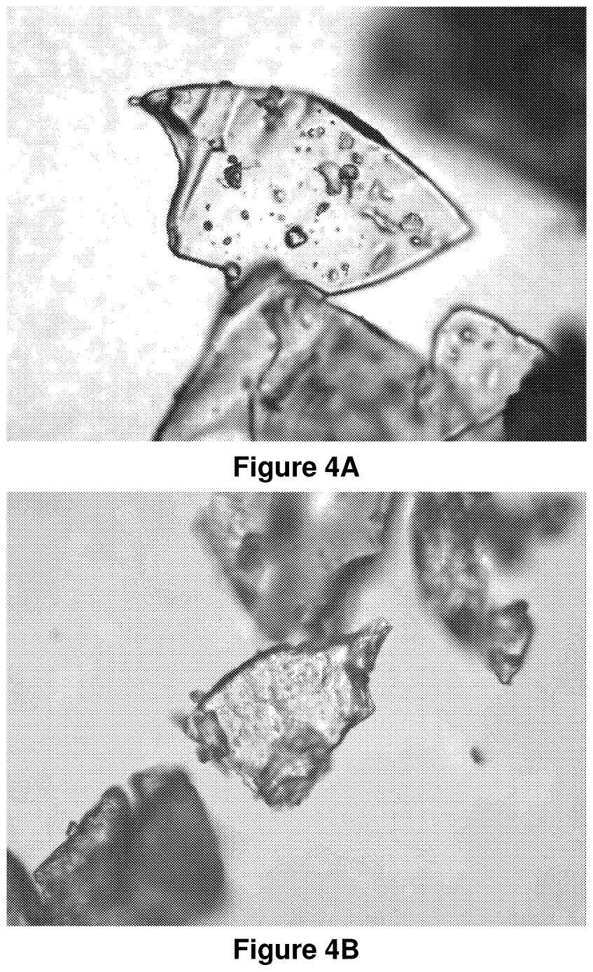

In some forms, the glucosamine orotate salt is a white powder that is stable at room temperature in open air. In these forms, the glucosamine orotate salt may be in a crystal form. For example, the glucosamine orotate salt is in a tetrahydrate crystal form, as illustrated in FIGS. 4A and 4B. As demonstrated in Example 6 below, the HPLC analysis revealed the stoichiometric inclusion of four water molecules per molecule of glucosamine orotate; and the microscopic images showed the crystal form of the glucosamine orotate salt. The glucosamine orotate tetrahydrate represents the first report of stoichiometric hydrate salt of glucosamine. Without being bound to any theories, one factor underlying the stability of glucosamine orotate salt may be that the crystalline lattice of glucosamine orotate includes water molecules present in stoichiometric whole numbers of water molecules per molecule of glucosamine orotate, and the water molecules may fill holes in the crystal lattice structure of glucosamine orotate powder, resulting in stable glucosamine orotate tetrahydrate. Nonstoichiometric hydrates of crystals also are known, though stoichiometric hydrates are generally more stable, and hydrate content can be affected by heating, resulting in alterations in crystal lattice structure.

The glucosamine orotate salt can also be in other hydrate crystal forms, such as a dihydrate crystal form or 0.8 hydrate crystal form. For example, the glucosamine orotate salt is in a tetrahydrate crystal form, a dihydrate crystal form, and/or a 0.8 hydrate crystal form.

In some forms, the glucosamine orotate salt in a hydrate crystal and/or amorphous form is an isolated compound, such as D-glucosamine orotate salt.

In some forms, the glucosamine orotate salt in tetrahydrate crystal form is an isolated compound, such as D-glucosamine orotate salt in tetrahydrate crystal form, designated as Glucosamine Orotate·4H₂O.

In some forms, the glucosamine orotate salt in dihydrate crystal form is an isolated compound, such as D-glucosamine orotate salt in dihydrate crystal form, designated as Glucosamine Orotate·2H₂O.

In some forms, the glucosamine orotate salt in 0.8 hydrate crystal form is an isolated compound, such as D-glucosamine orotate salt in 0.8 hydrate crystal form, designated as Glucosamine Orotate·0.8H₂O.

In some forms, the glucosamine orotate salt may be in an amorphous form, such as a tetrahydrate amorphous form, a dihydrate amorphous form, and/or a 0.8 hydrate amorphous form.

In some forms, the glucosamine orotate salt may be in a crystal form and/or an amorphous form, such as a tetrahydrate crystal form, a tetrahydrate amorphous form, a dihydrate crystal form, a dihydrate amorphous form, a 0.8 hydrate crystal form, and/or 0.8 hydrate amorphous form.

In some forms, the glucosamine orotate salt may be in an anhydrous form, such as an anhydrous crystal form and/or an anhydrous amorphous form.

In some forms, the glucosamine orotate salt may be in a hydrate form, optionally in different hydration states, and/or an anhydrous form, wherein the hydrate from and the anhydrous form each can be in crystal form and/or amorphous form.

In some forms, the glucosamine orotate salt in an anhydrous crystal and/or amorphous form is an isolated compound, such as D-glucosamine orotate salt.

The glucosamine orotate salt allows co-delivery of two precursors, a uridine nucleotide precursor (i.e., orotate) and glucosamine, to achieve augmentation of intracellular UDP-sugars for GAG and HA synthesis. The two precursors are provided in a single salt, without the need for inorganic counter ions that may affect ion homeostasis. The molecular mass of glucosamine is approximately 179 g/mol. In contrast, two molecules of glucosamine complexed with one sulfate and two sodium chlorides, a stable crystalline form widely used for treatment of osteoarthritis pain, has a formula mass of about 573 g/mol. Thus, about 35% of the molar mass of glucosamine sulfate-NaCl crystal contains the inorganic ions sulfate and NaCl, which may limit tolerable doses of oral glucosamine sulfate, as sodium sulfate has osmotic activity that can result in laxative effects.

The glucosamine orotate salt provided herein has a high water solubility (e.g., a water solubility of at least 1 g/mL, and up to 1.56 g/mL, at a temperature from 25 °C to 27 °C and under a pressure of 1 atm), which is approximately one order of magnitude greater than previously reported water solubilities for orotate salts, such as arginine orotate, lysine orotate, potassium orotate, and meglumine (methylglucosamine, which, unlike glucosamine, is a secondary amine and does not participate in glycosaminoglycan synthesis or other pharmacological activities of glucosamine) orotate; and approximately three orders of magnitude higher than the water solubility of orotic acid or its metal salts, such as sodium orotate, potassium orotate, magnesium orotate, or lithium orotate. Optionally, the glucosamine orotate salt has a water solubility that is about four orders of magnitude higher than the water solubility of free orotic acid. The high water-solubility of glucosamine orotate salt may result in improved bioavailability when compared to free orotic acid (with a solubility of about 0.1 mg/mL in water) and poorly soluble orotate salts, which may limit the rate and extent of dissolution and absorption after oral administration.

Further, the glucosamine orotate salt has an improved taste (neutral-sweet) compared to glucosamine, which has an unpleasant bitter taste, and orotate, which has a sour taste, separately.

### A. Preparation

Methods of producing glucosamine orotate salt are disclosed. Previously reported organic and inorganic cation salts of orotic acid (arginine orotate, lysine orotate, potassium orotate, meglumine orotate, etc.) are readily produced by mixing a suspension of orotic acid in water with the respective cationic component (e.g., arginine, lysine, potassium, or meglumine), with heating and stirring. As the salts are formed, the orotate goes into solution and the salts are then isolated by crystallization during cooling. As described in Example 1, this method did not produce glucosamine orotate salt (other than at most a negligible amount). In contrast, the methods disclosed herein are successful in preparing glucosamine orotate salt.

In some forms, the methods for producing glucosamine orotate salt involve combining glucosamine free base with orotic acid to form the glucosamine orotate salt. The glucosamine free base can be prepared using any suitable method, such as using ion exchange chromatography, precipitation, or a method described in U.S. Pat. No. 7,622,576B1, the disclosure of which is incorporated herein by its entirety.

### 1. Ion Exchange Chromatography

In some forms, the methods for producing glucosamine orotate salt use ion exchange chromatography to generate transient glucosamine free base from glucosamine hydrochloride or other suitable glucosamine salts, where the glucosamine free base combines with orotic acid to produce the glucosamine orotate salt. The methods using ion exchange chromatography do not introduce potentially hazardous reagents into the process and are scalable without producing large amounts of hazardous side products.

a. Passing Ion Exchange Column

In some forms, the method for preparing glucosamine orotate salt using ion exchange chromatography includes: (i) passing glucosamine hydrochloride through a column and into a suspension of orotic acid to form the glucosamine orotate salt. Typically, glucosamine free base is generated as the glucosamine hydrochloride passes through the ion exchange column, which in turn forms an ionic bond with orotic acid. In some forms, the molar ratio of the glucosamine hydrochloride to orotic acid is preferably about 1:1. For example, the molar ratio of the glucosamine hydrochloride to orotic acid ranges from 0.8:1 to 1.1:0.9, such as 0.8:1, 0.85:1, 0.9:1, 1:1.1, 1:1, 1:0.9, 1:0.85, 1.1:1, 1.2:1, 1.2:1.1, or 1.1:0.9. Optionally, the suspension of orotic acid is under stirring during step (i). The stirring is optionally performed using any suitable technique known in the art, such as by using magnetic stir bars or rotating paddles.

The column through which the glucosamine hydrochloride is passed is optionally an ion exchange column that contains an ion exchange resin loaded therein. Typically, the ion exchange resin capable of removing chloride ion from glucosamine hydrochloride to produce glucosamine free base. Any suitable base anion exchange resin is useful in the column, such as those made from acrylic gel (e.g., Amberlite™ IRA-67, Amberlite™ FPA-53) or macroporous styrene-divinylbenzene copolymer matrix (Dowex™ Marathon™ WBA-2, Dowex™-66). For example, the anion exchange resin used in the column in step (i) is a resin made from acrylic gel, such as Amberlite™ IRA-67.

Techniques for passing a compound through an ion exchange column are known. For example, step (i) is performed by (a) loading an aqueous solution of glucosamine hydrochloride onto the column, and (b) washing the column with deionized water. The aqueous solution of glucosamine hydrochloride is prepared by dissolving glucosamine hydrochloride in a suitable aqueous solvent, such as deionized water. The molar amount of the glucosamine hydrochloride in the aqueous solution is the same as the molar amount of the orotic acid in the suspension. After loading the aqueous solution of glucosamine hydrochloride, the column is washed with a suitable amount of eluent (e.g., deionized water), such as by continuously flowing deionized water through the column at a suitable rate. In some forms, the amount of deionized water and/or flow rate is determined based on the pH of the eluate. The suitable pH of the eluate indicating the end point is about 6-7.

The suspension of orotic acid is prepared by mixing orotic acid in water or in a suitable organic solvent (such as a volatile organic solvent), with or without water. For example, the orotic acid is mixed with the organic solvent under stirring. Optionally, the organic solvent is miscible with deionized water. Examples of organic solvents that are suitable for forming the orotic acid suspension include, but are not limited to, methanol, ethanol, 2-propanol, acetone, acetonitrile, dimethyl sulfoxide (DMSO), tetrahydrofuran (THF), propylene glycol, formamide, and N,N-dimethylformamide (DMF). In some forms, the organic solvent for forming the orotic acid suspension contains methanol and/or ethanol. For example, the organic solvent for forming the orotic acid suspension is methanol, ethanol, or a mixture thereof. For example, the organic solvent for forming the orotic acid suspension is methanol.

The eluate is collected into a container then combined with the suspension of orotic acid or directly into the suspension of orotic acid. For example, the eluate is directly collected into a container containing the suspension of orotic acid. Upon mixing the eluate and orotic acid suspension, the glucosamine orotate salt is formed. The salt-forming reaction is carefully controlled, as free glucosamine without a counter ion for its amino group is unstable in solution, and can undergo oxidative decomposition. For example, the eluate is collected dropwise directly into the suspension of orotic acid. Typically, during the addition of the eluate, the suspension becomes a clear homogeneous solution, indicating improved water solubility of the glucosamine orotate salt produced therein compared to orotic acid.

b. Optional Steps

Optionally, the method for preparing glucosamine orotate salt further includes (c) washing the ion exchange resin and (d) loading the washed ion exchange resin into the column; and/or (f) isolating the glucosamine orotate salt, optionally the isolation is performed by evaporation, optionally at room temperature or with warming, such as from about 25 °C to 50 °C, about 30 °C to 50 °C, about 35 °C to 50 °C, about 40 °C to 50 °C, about 25 °C to 45 °C, or about 25 °C to 35 °C, of the deionized water and organic solvent under vacuum.

In some forms, the method for preparing glucosamine orotate salt further includes, prior to step (i), (c) washing the ion exchange resin and (d) loading the washed ion exchange resin into the column. The ion exchange resin is washed using any suitable techniques. For example, the ion exchange resin is stirred in a suitable amount of deionized water, allowed to settle for a suitable amount of time, and then the water layer is decanted. The process is repeated one or more times until the water layer appears clear.

In some forms, the method for preparing glucosamine orotate salt further includes, after step (i), (f) isolating the glucosamine orotate salt from the solution. The isolation is performed using any suitable techniques, such as drying. For example, the isolation is performed by evaporation, optionally at room temperature or with warming, such as from about 25 °C to 50 °C, bout 30 °C to 50 °C, about 35 °C to 50 °C, about 40 °C to 50 °C, about 25 °C to 45 °C, or about 25 °C to 35 °C, of the deionized water and organic solvent in the solution under vacuum, optionally in the presence of a desiccant such as phosphorous pentoxide, to produce isolated glucosamine orotate salt as a solid. For example, the isolation is performed by precipitating glucosamine orotate salt from the solution using a suitable volume of an organic solvent, in which glucosamine orotate salt is poorly soluble, such as methanol or ethanol.

In some forms, the number of water molecules in the glucosamine orotate salt is controlled by the drying time period, such as under evaporation optionally at room temperature or with warming, such as at a temperature from about 25 °C to 50 °C, such as about 30 °C to 50 °C, about 35 °C to 50 °C, about 40 °C to 50 °C, about 25 °C to 45 °C, or about 25 °C to 35 °C. For example, the number of water molecules in the glucosamine orotate salt is varied by varying the drying time period in a range from about 7 days to about 14 days or from about 7 days to about 28 days, such as 7 days, 14 days, 15 days, 16 days, 17 days, 18 days, 19 days, 20 days, or 21 days, under vacuum at room temperature and optionally in the presence of a desiccant, such as phosphorous pentoxide. For example, the tetrahydrate crystal form of the glucosamine orotate salt is produced by evaporating the glucosamine orotate salt under vacuum for 7 days at room temperature and optionally in the presence of a desiccant, such as phosphorous pentoxide, while the dihydrate and/or 0.8 hydrate crystal form(s) of the glucosamine orotate salt is produced by evaporating the glucosamine orotate salt under vacuum for 14 days or more (e.g., from 14 days to 28 days) at room temperature and optionally in the presence of a desiccant, such as phosphorous pentoxide.

### 2. Other Methods for Generating Glucosamine Free Base

Besides ion exchange chromatography, the glucosamine free base can be prepared using alternative methods, which can then be combined with orotic acid to form glucosamine orotate salt. Exemplary alternative methods are described in U.S. Pat. No. 7,622,576B1.

For example, glucosamine free base can be prepared by reacting glucosamine hydrochloride (or another halide salt) with a lithium base (including but not limited to lithium hydroxide, lithium methoxide, lithium ethoxide, or lithium isopropoxide) in an alcohol with 1 to 4 carbon atoms (including but not limited to methanol, ethanol, isopropanol), with lithium hydroxide as the preferred reagent and methanol as the preferred solvent. While stirring at ambient temperature for 2 hours, the reaction product, glucosamine free base, precipitates and is isolated by filtration and washed with more alcohol. The resultant glucosamine free base is then used to prepare glucosamine orotate by addition to a stirred suspension of orotic acid in methanol, ethanol, or water, or mixtures of one or more of these solvents, followed by removal of the solvent under vacuum.

Alternatively, glucosamine free base can be prepared by treatment of glucosamine hydrochloride with an ethanolic solution of a tertiary base such as triethanolamine. Triethylamine hydrochloride is then filtered off and glucosamine free base is recovered from the reaction mixture. The glucosamine free base is then reacted with a suspension of orotic acid methanol, ethanol, or water, or mixtures of one or more of these solvents, followed by removal of the solvent under vacuum.

In some other forms, the methods for producing glucosamine orotate salt involve combining a silver salt of orotate with an aqueous solution of glucosamine hydrochloride to form the glucosamine orotate salt. Silver chloride (AgCl) is insoluble in water, so that it precipitates, yielding glucosamine free base and orotic acid as ion pairs that form glucosamine orotate in solution, which is recovered by removal of solvent under vacuum.

### B. Properties

The glucosamine orotate salt is highly water soluble and has an intrinsic acidic pH in aqueous solution. The glucosamine orotate salt also has a high purity and high stability.

### 1. Water Solubility

The glucosamine orotate salt has a high water solubility (e.g., a water solubility of at least 1 g/mL, and up to about 1.56 g/mL, at a temperature from 25 °C to 27 °C and under a pressure of 1 atm).

The water solubility of glucosamine orotate salt is approximately one order of magnitude greater than previously reported water solubilities for orotate salts (such as arginine orotate, lysine orotate, potassium orotate, and meglumine orotate), the highest reported water solubility of which is about 5% (1:20 w/v). For example, the reported water solubility for meglumine orotate salt (N-methylglucosamine orotate) is 5% (1:20 w/v). Solubility of potassium orotate is 1:500 w/v or 0.2% in water. The solubility of free orotic acid in water is approximately 0.1 mg/ml or 1:1000 w/v. Micronization of orotic acid increases its solubility by approximately 13-fold, to about 1.3%. In contrast, the solubility of glucosamine orotate salt reached 1.56:1 w/v or 156%, with more glucosamine orotate salt than water in the solution. The water solubility of glucosamine orotate salt is therefore far greater than that of other reported orotate salts.

### 2. Bioavailability

The high water solubility of glucosamine orotate salt may result in improved bioavailability compared to free orotic acid (with a solubility of about 0.1 mg/mL in water) and poorly soluble orotate salts, where slow dissolution after administration may limit the rate and extent of absorption. For example, as demonstrated in Example 4 below, glucosamine orotate displayed a higher Cₘₐₓ (maximum concentration achieved) and a higher AUC (“area under the curve”, the total amount measured in plasma integrated over time) than did each of magnesium orotate and orotic acid following oral administration to mice (Table 3). Further, glucosamine orotate yielded a higher concentration of circulating orotate at the 4-hour time point than did each of magnesium orotate and orotic acid following oral administration to mice (Table 3).

3. pH

The glucosamine orotate salt has an intrinsic pH of about 4, when measured in the form of a 14.2% water solution of glucosamine orotate salt. The pH of a water solution of glucosamine orotate salt is capable of being measured using a known technique, such as a calibrated pH meter or pH paper. A pH of about 4 of the glucosamine orotate salt is suitable for orally administered liquid formulations.

### 4. Purity

The glucosamine orotate salt has a high purity (e.g., at least 80%, at least 90%, or at least 99.8%). A person of skill in the art would understand that the glucosamine orotate salt may also contain impurities.

“Purity” refers to the percentage of the glucosamine orotate salt present with respect to any additional impurity or impurities. For example, when the glucosamine salt has a purity of 99.8%, one or more impurities constitute the remaining 0.2%. Impurities present in the glucosamine orotate salt may include organic impurities (e.g., starting materials, by-products, intermediates, degradation products), inorganic impurities, and/or residual solvents, which may be introduced in the manufacturing process or during storage of the glucosamine salt. In some forms, one or more degradation products of glucosamine (e.g., 5-(hydroxymethyl) furfural (HMF)) are impurities that are potentially present in the glucosamine orotate salt. Analytical methods for testing for impurities (e.g., thin-layer chromatography) and other potential impurities are known and further described in U.S. Food and Drug Administration. Guidance for Industry, Q3A Impurities in New Drug Substances. June 2008, International Conference on Harmonization (ICH) Q2B, “Validation of Analytical Procedures: Methodology,” Federal Register, Vol. 62, No. 96, 1997, pp. 27463-27467, and Food and Drug Administration. (2024). Q2 (R2) Validation of Analytical Procedures: Guidance for Industry. Silver Spring, MD: U.S. Department of Health and Human Services, FDA.

In some forms, the glucosamine orotate salt has a purity of at least 80%, at least 85%, at least 90%, at least 95%, at least 98%, or at least 99% or from 85% to 100%, from 90% to 100%, from 95% to 100%, from 98% to 100%, or from 99% to 100%.

In some forms, the glucosamine orotate salt has a purity of at least 99.8%.

### 5. Stability

The glucosamine orotate salt has high stability. For example, the glucosamine orotate salt does not change shapes, morphology, appearance, sizes, and/or the percentage of weight change is less than 1% for at least 1 month, at least 3 months, at least 6 months, at least 1 year, at least 2 years, or at least 3 years, at standard pressure, at a temperature ranging from about 3 °C to about 45 °C, and at a humidity of ≤10% or ranging from about 30% to about 60%. Methods for measuring the stability of the glucosamine orotate salt are known and include methods such as chromatography (e.g., high-performance liquid chromatography (HPLC) using an evaporative light scattering detector, microscope, etc.).

In some forms, the glucosamine orotate salt has higher stability than other glucosamine salts, such as glucose sulfate salt. Glucosamine is typically vulnerable to degradation which is a problem with other glucosamine salts such as glucosamine sulfate. Glucosamine hydrochloride is the only stable form currently on the market. For example, when glucosamine sulfate is stabilized with sodium chloride, mixed glucosamine hydrochloride and sodium sulfate crystals are formed instead of glucosamine sulfate crystals.

In some forms, the high stability of the glucosamine orotate salt permits storage of the glucosamine orotate salt for at least 6 months, at least 1 year, at least 2 years, or at least 3 years under ambient conditions (e.g., from about 15 °C up to 30 °C or from about 15 °C to about 25 °C, such as at room temperature, at 30% to 60% humidity) or in fridge (e.g., from about 3 °C to about 10 °C at 30% to 60% humidity) without a decrease in purity, such as measured by HPLC.

In some forms, the glucosamine orotate salt is stable at temperatures higher than ambient temperatures (e.g., from above 30 °C to about 45 °C at 30% to 60% humidity) and can be stored for at least 1 month, at least 3 months, at least 4 months, at least 5 months, or at least 6 months without a decrease in purity, such as measured by HPLC.

In some forms, the glucosamine orotate salt is stable at temperatures higher than ambient temperatures (e.g., from above 30 °C to about 45 °C at ≤10% or 30% to 60% humidity) for a period from 1 day to 30 days (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) without a decrease in purity, such as measured by HPLC. For example, the glucosamine orotate salt is stable at an elevated temperature of about 45 °C at ≤10% humidity for 5 days, 10 days, or 14 days without a decrease in purity, such as measured by HPLC.

In some forms, the glucosamine orotate salt in crystal form has high stability, which permits storage of the glucosamine orotate salt for at least 6 months, at least 1 year, at least 2 years, or at least 3 years under ambient conditions (e.g., from about 15 °C up to 30 °C or from about 15 °C to about 25 °C, such as at room temperature, at 30% to 60% humidity) or in fridge (e.g., from about 3 °C to about 10 °C at 30% to 60% humidity) without a decrease in crystallinity, such as measured using a microscope (e.g., OMAX™ microscope).

In some forms, the glucosamine orotate salt in crystal form is stable at temperatures higher than ambient temperatures (e.g., from above 30 °C to about 45 °C at 30% to 60% humidity) and can be stored for at least 3 months, at least 4 months, at least 5 months, or at least 6 months without a decrease in crystallinity, such as measured using a microscope (e.g., OMAX™ microscope).

In some forms, the glucosamine orotate salt in crystal form is stable at temperatures higher than ambient temperatures (e.g., from above 30 °C to about 45 °C at ≤10% or 30% to 60% humidity) for a period from 1 day to 30 days (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days) without a decrease in crystallinity, such as measured using a microscope (e.g., OMAX™ microscope). For example, the glucosamine orotate salt in crystal form (such as tetrahydrate crystal form, dihydrate crystal form, and/or 0.8 hydrate crystal form) is stable at an elevated temperature of about 45 °C at ≤10% humidity for 5 days, 10 days, or 14 days without a decrease in crystallinity, such as measured using a microscope (e.g., OMAX™ microscope).

### II. Compositions

Compositions containing glucosamine orotate salt are described herein. The glucosamine in the salt is optionally in an isomer form. For example, the glucosamine orotate salt in the composition is D-glucosamine orotate salt. In some forms, the glucosamine orotate salt in the compositions is an isolated glucosamine orotate salt. For example, the isolated glucosamine orotate salt in the composition is D-glucosamine orotate salt.

The compositions include pharmaceutical compositions, cosmetic compositions, or nutraceutical (dietary supplement) compositions. Pharmaceutical, cosmetic, and nutraceutical compositions contain the glucosamine orotate salt and, optionally, one or more suitable carrier(s) and/or excipient(s). The excipients used in the compositions containing glucosamine orotate salt are typically non-ionic to prevent disruption of the ionic bond of glucosamine orotate salt. In some forms, the compositions contain the glucosamine orotate salt with high purity and high stability and, optionally, one or more suitable carrier(s) and/or excipient(s). In some forms, the compositions contain the glucosamine orotate salt in crystal form, such as tetrahydrate crystal form, dihydrate crystal form, and/or 0.8 hydrate crystal form.

In some forms, the composition (e.g., pharmaceutical composition or nutraceutical composition) is in a suitable form, such as a pill, capsule, tablet, dry powder, a soft or hard chew, a gummy, or liquid form, for oral administration to a subject. For example, the composition is in a liquid form (e.g., a ready-to-drink liquid), dry powder form (e.g., a drink mix), capsule form, or tablet form, that is suitable for oral administration. In some forms, the improved solubility of glucosamine orotate salt allows for liquid formulations (e.g., liquid pharmaceutical composition or nutraceutical composition) thereof for intravenous or subcutaneous injection or infusion. In some forms, composition containing glucosamine orotate salt (e.g., pharmaceutical composition or cosmetic composition) is in a suitable form for topical application to the skin.

In some forms, the glucosamine orotate salt in the composition is in an effective amount to restore, improve, or support the health of blood vessels; to improve maintenance or repair of cartilage in joints or tendons; and/or to restore, improve, or support the health of liver. In some forms, the glucosamine orotate salt in the composition is in an effective amount to (1) promote synthesis of glycosaminoglycans (GAGs) and hyaluronan (HA), (2) reduce local and/or systemic inflammation, and/or (3) improve or maintain mitochondrial function, thereby restoring, improving, or supporting the health of blood vessels, improving maintenance or repair of cartilage in joints or tendons, and/or restoring, improving, or supporting the health of liver in a subject. In some forms, the glucosamine orotate salt in a topical composition is in an effective amount to support GAG and HA synthesis locally in the skin. In some forms, the glucosamine orotate salt in the composition is in an effective amount to improve or maintain the health of the endothelial glycocalyx of a subject. In some forms, the glucosamine orotate salt in the composition is in an effective amount to reduce and/or ameliorate one or more symptom(s) (e.g., chronic inflammation) associated with a mitochondrial disease or disorder of a subject. In some forms, the glucosamine orotate salt in the composition is in an effective amount to reduce and/or ameliorate one or more symptom(s) (e.g., chronic inflammation) associated with a disease or disorder associated with cGAS-STING activation, such as trauma, infection, or a disease or disorder that is at least partially a pathophysiological consequence of cGAS-STING activation (e.g., cytokine storm).

### A. Orally Administered Pharmaceutical Compositions

Pharmaceutical compositions that contain glucosamine orotate salt in a form suitable for oral administration to a mammal are disclosed. The pharmaceutical composition may include one or more suitable carriers and/or one or more suitable excipients for oral administration, such as in solid or liquid forms. In the forms, the glucosamine orotate salt is provided as pure compound, optionally in tetrahydrate, dihydrate, and/or 0.8 hydrate crystal form, without any excipients or carriers. Representative carriers and excipients include water, ethanol, polyethylene glycol, propylene glycol, bulking agents (e.g., microcrystalline cellulose, silicon dioxide, etc.), drying agents, diluents, pH modifying agents, preservatives, antioxidants, suspending agents, wetting agents, viscosity modifiers, tonicity agents, and stabilizing agents, and a combination thereof.

The pharmaceutical composition containing glucosamine orotate salt is in a form suitable for oral administration to a subject, such as a mammal (i.e. an oral formulation). Oral administration typically involves swallowing, so that the glucosamine orotate salt containing pharmaceutical composition enters the gastrointestinal tract, or buccal or sublingual administration may be employed by which the pharmaceutical composition containing glucosamine orotate salt enters the blood stream directly from the mouth.

Compositions suitable for oral administration include solid compositions such as tablets, capsules, powders, lozenges (including liquid-filled lozenges), chews (soft or hard), gummy, dry blend, multi- and nano-particulates, gels, films, ovules, and/or dry powder sprays.

Liquid compositions for oral administration include suspensions, solutions, syrups, and/or elixirs. Optionally, the liquid compositions are delivered in soft or hard capsules. Liquid compositions for oral administration may also be prepared by the reconstitution of a solid (e.g., a drink mix), for example, from a sachet shortly before ingestion.

Optionally, the pharmaceutical composition is in the form of a liquid, such as a solution or a suspension, and contains the glucosamine orotate salt in an aqueous medium, optionally with one or more suitable excipients for the liquid composition, such as water, ethanol, polyethylene glycol, propylene glycol, methylcellulose, a suitable oil, one or more emulsifying agents, and/or suspending agents. When provided in a liquid form, the liquid pharmaceutical composition may be a ready-to-drink liquid.

When in the form of a liquid composition, in addition to glucosamine orotate salt, the liquid composition may contain buffering agent, antioxidants, colorants, flavoring agents, preservatives, or taste-masking agents, or a combination thereof. Exemplary liquid unit dose formulations have a unit dose volume of up to about 30 mL, and contain from 1 gram to 6 grams of glucosamine orotate salt, which corresponds to a range of about 3% to 25% w/v glucosamine orotate salt with a corresponding 75-97% w/v liquid carrier, which optionally includes flavorants, thickeners, and/or other suitable excipients.

Optionally, the pharmaceutical composition is in a solid form, such as dry powder form, and contains the glucosamine orotate salt and optionally one or more suitable excipients for a solid composition, such as one or more drying agent(s). The solid pharmaceutical composition, such as dry powder (e.g., a drink mix), may be added to water or other suitable liquid for oral administration.

Optionally, the glucosamine orotate salt is included in a fast-dissolving and/or fast-disintegrating dosage form. Optionally, the glucosamine orotate salt is provided as dry powder form, such as produced by freeze drying, spray drying, or micronization.

For a solid composition, such as pill, tablet, capsule, chew (soft or hard), gummy, or dry powder composition, in addition to glucosamine orotate salt, tablets may contain drying agents, disintegrants, binders, diluents, surface active agents, lubricants, glidants, antioxidants, colorants, flavoring agents, preservatives, or taste masking agents, or a combination thereof.

Examples of suitable drying agents for forming a solid composition, such as a pill, tablet, capsule, chew (soft or hard), gummy, or dry powder composition containing glucosamine orotate salt include, but are not limited to, silica gel and molecular sieves.

Examples of suitable disintegrants for forming a solid composition, such as a pill, tablet, capsule, chew (soft or hard), gummy, or dry powder composition containing glucosamine orotate salt include, but are not limited to, sodium starch glycolate, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant has a concentration in a range from about 1 weight % to about 25 weight %, from about 5 weight % to about 20 weight % of the composition containing glucosamine orotate salt.

Binders are generally used to impart cohesive qualities to a solid composition. Suitable binders for forming a solid composition, such as a pill, tablet, capsule, chew (soft or hard), gummy, or dry powder composition containing glucosamine orotate salt include, but are not limited to, microcrystalline cellulose, gelatin, sugars, polyethylene glycol, natural and synthetic gums, polyvinylpyrrolidone, pregelatinized starch, hydroxypropyl cellulose, and hydroxypropyl methylcellulose.

Suitable diluents for forming a solid composition, such as a pill, tablet, capsule, chew (soft or hard), gummy, or dry powder composition containing glucosamine orotate salt include, but are not limited to, lactose (as, for example, the monohydrate, spray-dried monohydrate or anhydrous form), mannitol, xylitol, dextrose, sucrose, sorbitol, microcrystalline cellulose, and starch.

Solid compositions, such as pill, tablet, capsule, chew (soft or hard), gummy, or dry powder compositions containing glucosamine orotate salt may also contain surface active agents, such as sodium lauryl sulfate and polysorbate 80, and glidants, such as silicon dioxide and talc. When present, surface active agents have a concentration in a range from about 0.2 weight % to 5 weight % of the composition.

Other possible excipients included in a solid composition, such as a pill, tablet, capsule, chew (soft or hard), gummy, or dry powder composition containing glucosamine orotate salt include glidants (e.g. Talc or colloidal anhydrous silica at about 0.1 weight % to about 3 weight % of the composition), antioxidants, colorants, flavoring agents, preservatives, and taste-masking agents. When present, glidants have a concentration in a range from about 0.2 weight % to 1 weight % of the composition.

In some instances, the pharmaceutical composition is in the form of a tablet. Exemplary tablets include glucosamine orotate salt, and one or more drying agents, and optionally one or more binders, diluents, disintegrants, and/or lubricants. An exemplary tablet composition contains up to about 99 weight % (such as up to about 95 weight %, up to about 90 weight %, up to 85 weight %, or up to about 80 weight %) of glucosamine orotate salt described herein, from about 1 weight % to about 50 weight % drying agent, and optionally from about 1 weight % to about 90 weight % binder, from about 0 weight % to about 85 weight % diluent, from about 1 weight % to about 10 weight % disintegrant, and from about 0.1 weight % to about 10 weight % lubricant.

Pills or tablets, including glucosamine orotate salt and one or more suitable excipients, may be compressed directly or by roller to form pills or tablets. Dry blends or portions of the blends may alternatively be wet-, dry-, or melt-granulated, melt-congealed, or extruded before tableting.

The final solid composition, such as pill, tablet, capsule, chew (soft or hard), gummy, or dry powder composition may contain one or more layers and may be coated or uncoated. Solid compositions containing glucosamine orotate salt for oral administration may be formulated to be immediate and/or to provide modified release. Modified release compositions include delayed, sustained, pulsed, controlled, targeted and/or programmed release formulations.

### B. Orally Administered Nutritional Supplement Compositions

The compounds from which glucosamine orotate salt is prepared are both endogenous biochemicals and are found in many foods. The production of glucosamine orotate salt does not alter the covalent bonding of its constituents. Therefore, glucosamine orotate salt falls under regulatory guidelines as a nutritional supplement or “nutraceutical” (a food-derived substance with potential pharmacologic activity). Pharmaceutical compositions, including their carriers and excipients, disclosed above can be used as nutraceutical compositions. Powder of pure glucosamine orotate salt, without an excipient, is also a form of a nutraceutical composition, similar to the form of common nutraceuticals such as creatine, provided in bulk to be mixed into liquids. Nutraceutical compositions can also contain mixtures of two or more active agents, including botanical materials and/or extracts. Nutraceutical compositions containing glucosamine orotate salt in combination or co-administered with any other active ingredients or excipients can be selected for complementary activity in a given health condition.

Optionally, in addition to glucosamine orotate salt, the nutraceutical compositions further contain one or more additional active agents. In some forms, the additional active agent(s) in the composition can act additively or collectively with glucosamine orotate salt to achieve a therapeutic or prophylactic treatment goal.

The specific additional active agent(s) in the composition can be selected based on the intended therapeutic goals. For example, for protection and repair of the vascular endothelial glycocalyx, in addition to glucosamine orotate salt, the nutraceutical composition includes one or more flavonoid compounds and/or one or more arginine source(s). For example, for protection of the liver, in addition to glucosamine orotate salt, the nutraceutical composition includes one or more nitric oxide elevating compound(s).

Suitable flavonoid compounds for use in the compositions, such as a nutraceutical composition, include, but are not limited to diosmin, hesperidin, naringenin, narengin, hesperetin, rutin, tangereretin, eriodictyol, and other related citrus flavones or citrus flavanones. Such botanical flavonoids are believed to have vasculoprotective and other beneficial health effects that are further described in Mahmoud, A. M. et al. (2019). Beneficial Effects of Citrus Flavonoids on Cardiovascular and Metabolic Health. Oxidative medicine and cellular longevity, 2019, 5484138.

Suitable nitric oxide elevating compounds for use in the composition, such as a nutraceutical composition, include, but are not limited to, arginine sources (e.g., arginine, citrulline), and plant extracts with high nitrate content (e.g., beet extract, arugula extract, etc.). Plant extracts with “high nitrate content” as used herein refers to extracts from plants that contain at least 100 mg of nitrate per 100 grams of plant matter. For example, arugula has ~480 mg nitrate per 100 grams.

In some forms, the composition, such as pharmaceutical composition, includes one or more nitric oxide elevating compounds. Nitric oxide elevating compounds suitable for use in pharmaceutical compositions include arginine sources and plant extracts as described above for nutraceutical compositions, as well as nitrate, isosorbide mononitrate, isosorbide dinitrate, nitroglycerin, nicorandil, molsidomine, and phosphodiesterase 5 (PDE5) inhibitors (e.g., sildenafil, vardenafil, or tadalafil).

Suitable arginine sources for use in the composition, such as a nutraceutical composition, include, but are not limited to, citrulline and arginine. Arginine (and indirectly, its precursor citrulline) regulate the production of the mediator Nitric Oxide, which is involved both in health of vascular endothelial cells and in their transmission of signals favoring vasorelaxation to their underlying smooth muscle, thereby improving blood flow and reducing risk of vascular occlusion and hypertension. Citrulline is converted to arginine after oral administration and can provide elevated circulating arginine concentrations better than does orally administered arginine.

In some forms, in addition to glucosamine orotate salt, the composition includes one or more citrus flavonoids and/or one or more nitric oxide elevating compounds. For example, in addition to glucosamine orotate salt, the composition includes one or more of the following active agents: diosmin, hesperidin, citrulline, and arginine. For example, in addition to glucosamine orotate salt, the composition includes: (1) diosmin and/or hesperidin; (2) citrulline and/or arginine; or a combination of (1) and (2). For example, in addition to glucosamine orotate salt, the composition includes diosmin and citrulline.

When glucosamine orotate is intended for treatment of liver inflammation associated with metabolic conditions, such as fatty liver or steatohepatitis, or liver inflammation secondary to inflammatory bowel disease, potential additional components of a dietary supplement product include carnitine or its acyl derivatives, including but not limited to, acetyl-carnitine and/or propionyl carnitine, in daily dosages ranging from 50 to 3000 milligrams. Carnitine aids in metabolism of fat in tissues including the liver by mediating fatty acid transport into mitochondria.

The one or more additional active agents can present in any suitable amounts in the composition. In some forms, the composition contains nitric oxide elevating compounds, such as sodium nitrate, beetroot juice, or beetroot concentrate or powder, in an amount to provide a nitrate dose of 4 to 12 mg/kg or 300 to 800 mg/day. In some forms, the composition contains 1 gram to 6 grams of arginine or citrulline or a combination thereof. In some forms, the composition alternatively or additional contains 0.1 gram to 1 gram of diosmin, hesperidin, or another citrus flavone or citrus flavanone, or a combination thereof.

### C. Parenterally Administered Pharmaceutical Compositions

In some forms, the pharmaceutical composition is in a form suitable for administration directly into the blood stream, into muscle, or into an internal organ. In some forms, the pharmaceutical composition is in a form suitable for local injections, for example, direct injection into joints or synovial fluid to support local cartilage maintenance or repair. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical composition containing glucosamine orotate salt is in a form suitable for intravenous administration or subcutaneous administration. In some forms, intravenous or subcutaneous administered pharmaceutical composition is suitable for treatment of acute conditions, for example, vascular endothelial glycocalyx deterioration that occurs after trauma or during infections (including sepsis, bacterial infections, fungal infections, and viral infections, such as infections with SARS-CoV2).

Parenteral compositions containing glucosamine orotate salt are typically liquid formulations, which may be a solution, a suspension, or an emulsion. In some forms, the parenteral compositions are aqueous solutions that optionally contain excipients such as salts, carbohydrates and pH buffering agents (e.g., from about pH 6.0 to about pH 7.4, from about pH 6.5 to about pH 7.0, but, for some applications, they may be more suitably formulated as a sterile aqueous solution or as a dried form to be used in conjunction with a suitable vehicle such as sterile, pyrogen-free water.

Exemplary solutions for intravenous or subcutaneous injection or infusion contain glucosamine orotate salt, water, and optionally salts or osmolytes, such as glucose or glycerol, to yield an adequately isotonic solution for systemic administration.

The preparation of parenteral compositions containing glucosamine orotate salt is typically under sterile conditions, for example, by lyophilization, which can be accomplished using standard pharmaceutical techniques known to those skilled in the art.

### D. Topically Administered Pharmaceutical Compositions

In some forms, the pharmaceutical composition is in a form suitable for topical application to the skin. In some forms, topical administration of pharmaceutical composition containing glucosamine orotate is suitable for local treatment of skin, for example, to support local synthesis of GAG and HA in thin or wrinkled skin. Such topical compositions can be in various forms suitable for direct application to the skin, such as gels, creams, ointments, or patches. Optionally, the topical compositions can be in suitable forms for delivery into the skin, such as using needles or arrays of microneedles.

When glucosamine orotate salt in the topical composition is administered directly to the external surface of the skin, the glucosamine orotate salt crosses the external surface of the skin and enters the underlying tissues.

Compositions for topical administration generally contain a dermatologically acceptable carrier and/or excipient that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, and the like). Examples of topical carrier systems useful in the topical compositions are described in the following four references all of which are incorporated herein by reference in their entirety: “Sun Products Formulary” Cosmetics & Toiletries, vol. 105, pp. 122-139 (December 1990); “Sun Products Formulary,” Cosmetics & Toiletries, vol. 102, pp. 117-136 (March 1987); U.S. Pat. No. 5,605,894 to Blank et al., and U.S. Pat. No. 5,681,852 to Bissett.

Compositions containing glucosamine orotate salt for topical administration may be formulated to be immediate and/or to provide modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of glucosamine orotate salt. Examples of such formulations include drug-coated stents and poly(dl-lactic-coglycolic) acid (PGLA) microspheres.

### E. Cosmetic Compositions

The disclosed compositions containing glucosamine orotate salt are formulated as cosmetic compositions, with, optionally, one or more additional active agents, one or more cosmetically acceptable excipients, or one or more cosmetically acceptable excipients, or combinations thereof. Carriers and excipients for the cosmetic compositions are formed with materials that are generally recognized as safe. Carriers suitable for dermatological applications, injections, implantations, oral administration, and other suitable routes administered to an individual without causing undesirable biological side effects or unwanted interactions. Representative carriers and excipients include solvents, diluents, pH modifying agents, preservatives, antioxidants, suspending agents, wetting agents, viscosity modifiers, tonicity agents, stabilizing agents, and combinations thereof. The carriers and excipients of the cosmetic composition can contain from about 5% to about 95% by weight of the composition, such as from about 10% to about 90% or from about 15% to about 85% by weight of the composition.

In some forms, the cosmetic compositions are formulated for topical administration. In some forms, the cosmetic composition contains glucosamine orotate salt and one or more dermatologically acceptable carrier(s) and/or excipient(s) as described above for the topical compositions.

In some forms, the cosmetic compositions are formulated for oral administration. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, soft or hard chews, gummies, troches or lozenges, cachets, pellets, powders, or granules or incorporation of the material into particulate preparations of polymeric compounds such as polylactic acid, polyglycolic acid, etc. or into liposomes. The cosmetic compositions may be prepared in liquid form, or may be in dried powder (e.g., lyophilized) form. Liposomal or proteinoid encapsulation may be used to formulate the cosmetic compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes, Chapter 10, 1979. Other forms provide liquid dosage forms for oral administration, including emulsions, solutions, suspensions, and syrups, which may contain other components such as inert diluents; adjuvants (e.g., wetting agents), emulsifying and suspending agents; and sweetening and flavoring agents.

### F. Dosages and Concentrations

Suitable doses and dosing regimens for the compositions disclosed herein (e.g., nutraceutical compositions, pharmaceutical compositions, and cosmetic compositions) deliver into the circulation or into target tissues effective amounts of glucosamine orotate salt for attaining one or more therapeutic goals, including, but not limited to, supporting the health of blood vessels; mitigating pain and joint damage due to osteoarthritis; reducing inflammation and injury in both local and systemic inflammations, such as SIRS (Systemic Inflammatory Response Syndrome), sepsis, or multiple organ failure syndrome; supporting the health of the liver (such as by protecting the liver against inflammatory damage, or reducing hepatic inflammation and/or local hepatic injury in a subject); reducing and/or ameliorating one or more symptom(s) (e.g., chronic inflammation) associated with a mitochondrial disease or disorder; and/or reducing and/or ameliorating one or more symptom(s) (e.g., chronic inflammation) associated with a disease or disorder related to cGAS-STING activation (such as trauma, infection, or disease/disorder that is at least partially a pathophysiological consequence of cGAS-STING activation, e.g., cytokine storm).

The term “treat”, “treatment”, or “treating” means to ameliorate or reduce or otherwise stop a disease, disorder or condition from progressing in an animal; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis.

In some forms, the doses of glucosamine orotate salt in the composition deliver into the circulation or into target tissues effective amounts of glucosamine orotate salt for promoting synthesis of glycosaminoglycans (GAGs) and hyaluronan (HA), reducing local and/or systemic inflammation, and/or improving or maintaining mitochondrial function, thereby attaining one or more therapeutic goals, including, but not limited to, supporting the health of blood vessels, mitigating pain and joint damage due to osteoarthritis, and reducing inflammation and injury in both local and systemic inflammations, such as SIRS, sepsis, or multiple organ failure syndrome; supporting the health of the liver (such as by protecting the liver against inflammatory damage, or reducing hepatic inflammation and/or local hepatic injury in a subject); reducing and/or ameliorating one or more symptom(s) (e.g., chronic inflammation) associated with a mitochondrial disease or disorder; and/or reducing and/or ameliorating one or more symptom(s) (e.g., chronic inflammation) associated with a disease or disorder associated with cGAS-STING activation (such as trauma, infection, or disease/disorder that is at least partially a pathophysiological consequence of cGAS-STING activation, e.g., cytokine storm).

In some forms, the doses of glucosamine orotate salt in the composition deliver into the circulation or into target tissues effective amounts of glucosamine orotate salt for promoting synthesis of glycosaminoglycans (GAGs) and hyaluronan (HA) and/or improving or maintaining mitochondrial function, thereby attaining one or more therapeutic goals, including, but not limited to, supporting the health of blood vessels, mitigating pain and joint damage due to osteoarthritis, and reducing inflammation and injury in both local and systemic inflammations, such as SIRS, sepsis, or multiple organ failure syndrome; supporting the health of the liver (such as by protecting the liver against inflammatory damage, or reducing hepatic inflammation and/or local hepatic injury in a subject); reducing and/or ameliorating one or more symptom(s) (e.g., chronic inflammation) associated with a mitochondrial disease or disorder; and/or reducing and/or ameliorating one or more symptom(s) (e.g., chronic inflammation) associated with a disease or disorder associated with cGAS-STING activation (such as trauma, infection, or disease/disorder that is at least partially a pathophysiological consequence of cGAS-STING activation, e.g., cytokine storm).

Generally, the concentration of glucosamine orotate salt in the composition is in a range from about 1 weight % to about 99 weight % or from about 10 weight % to about 99 weight %, such as from about 2 weight % to about 99 weight %, from about 2 weight % to about 90 weight %, from about 2 weight % to about 80 weight %, from about 2 weight % to about 60 weight %, from about 3 weight % to about 99 weight %, from about 3 weight % to about 90 weight %, from about 3 weight % to about 80 weight %, from about 3 weight % to about 60 weight %, from about 10 weight % to about 95 weight %, from about 10 weight % to about 90 weight %, from about 10 weight % to about 80 weight %, from about 20 weight % to about 60 weight %, from about 20 weight % to about 99 weight %, or from about 20 weight % to about 90 weight %. The specific concentration of glucosamine orotate salt in the composition depends on the specific therapeutic goal, the specific form of the composition, the presence of carriers and excipients, and the presence of any additional active agents. In some forms, solid oral compositions of relatively high-dose glucosamine orotate salt (e.g., in grams) are designed to minimize the amounts of excipients such as binders, drying agents, and the like in the compositions. For example, a capsule can encapsulate 100% glucosamine orotate salt within a gelatin or cellulosic capsule. For example, in nutraceutical compositions, the concentration of glucosamine orotate salt can be affected by the presence of other active agents (such as arginine/citrulline, diosmin, etc.) in the composition.

The composition is optionally provided in a dosage form. The dosage form refers to a physical form in which a drug or nutraceutical is manufactured or administered. Examples of dosage forms include tablets, capsules, powders, pills, chews (soft or hard), gummies, oral and injectable solutions, etc. A drug or nutraceutical may be available in various dosage forms.

Without being bound to any theories, the daily dose of glucosamine orotate salt is believed to overcompensate for feedback inhibition of de novo pyrimidine synthesis, and its conversion to UMP by UMP synthase (which catalyzes both phosphoribosylation of orotic acid with PRPP as a ribose donor, and decarboxylation of the resultant orotidine monophosphate to yield UMP) is not inhibited by orotic acid. Further, the daily dose of glucosamine orotate salt is not too much such that it is not entirely assimilated into uridine nucleotides but spills over into the urine, as excess orotate can crystallize in and obstruct renal tubules, as occurs in the genetic disease Hereditary Orotic Aciduria (“HOA”). HOA is a deficiency of UMP Synthase activity, resulting in an inability to convert orotic acid into UMP, causing systemic uridine nucleotide deficiency (with resultant anemia, developmental delays and failure to thrive) and accumulation of orotic acid in the blood and uridine.

Suitable dosages of glucosamine orotate salt in the composition generally range from about 2 grams per day up to about 8 grams per day, from about 2 grams per day up to about 6 grams per day, from about 2 grams per day up to about 4 grams per day, from about 3 grams per day up to about 8 grams per day, from about 3 grams per day up to about 6 grams per day, from about 4 grams per day up to about 8 grams per day, from about 4 grams per day up to about 6 grams per day, or from about 6 grams per day up to about 8 grams per day. The daily glucosamine orotate salt dose delivered to the subject can be in a single dosage form or divided into two or more doses per day, such as two or three doses per day. The specific dose of glucosamine orotate salt in the daily total dose depends on the specific therapeutic goal, target subject, specific administration route, specific form of the composition, etc.

In some forms, the composition containing glucosamine orotate salt is in a dosage form, such as any one of those described above, for administration to a subject, where the glucosamine orotate salt is present in the dosage form in an amount ranging from about 300 mg to about 8 g, from about 300 mg to about 4 g, from about 300 mg to about 1 g, from about 300 mg to about 500 mg, 1 g to about 8 g, from about 1 g to about 4 g, from about 2 g to about 8 g, from about 3 g to about 8 g, from about 1 g to about 6 g, from about 2 g to about 6 g, from about 3 g to about 6 g, or from about 4 g to about 6 g. The daily dose can be in a single dosage form or divided into two or more doses per day, such as two or three doses per day.

For example, the composition containing glucosamine orotate salt is in a dosage form, wherein the glucosamine orotate salt is present in the dosage form is in an amount ranging from about 1 g to about 8 g, such as from about 2 g to about 8 g, or from about 4 g to about 6 g that is administered once or twice daily. For example, the composition containing glucosamine orotate salt is in a dosage form, wherein the glucosamine orotate salt is present in the dosage form is in an amount ranging from 300 mg to 1 g or from 300 mg to 800 mg that is administered twice or three times daily. For example, the composition containing glucosamine orotate salt is in a dosage form, wherein the glucosamine orotate salt is present in the dosage form is in an amount ranging from 300 mg to 1 g or from 300 mg to 800 mg and multiple dosage forms are administered at once. For example, more than one capsule each containing 300 or 500 mg glucosamine orotate salt is administered at once, which can be repeated one or more times daily.

The doses of drug or nutraceutical found to be active in small laboratory mammals can be translated into predicted active and sufficient doses for human subjects by “allometric scaling” based on relative body surface areas with empirical validation across multiple drug or nutraceutical categories. Thus, a dose in mg/kg units in mice, for example 500 mg/kg, would translate to a dose of 500/12 mg/kg or about 41.7 mg/kg in a human subject. In a 70 kg human adult, 41.7 mg/kg of glucosamine orotate converts to an absolute dose of 70×41.7-2,919 mg, or very close to 3 grams. This dose corresponds closely to the mouse dose of 522 mg/kg that provided strong protection of the liver against inflammatory damage caused by bacterial endotoxin in Examples 5 and 6, below. Appropriate doses in human subjects of different body sizes are approximately proportional to body weight. Thus, a dose of 3 grams in a 70 kg adult corresponds to a dose of 1.5 grams in a 35 kg child.

The improved health of blood vessels and joint health, optionally as a result of the promoted synthesis of GAGs and HA and/or reduced local and/or systemic inflammation, achieved by the composition may be indicated by inspection of the appearance of cartilage and/or endothelial glycocalyx of the subject using an imaging tool compared to the appearance of the cartilage and/or endothelial glycocalyx prior to administration of the composition.

The health of cartilage and related factors in joint health can be assessed with imaging tools, such as X-rays, CT scans, or MRI, which can quantify the thickness and density of cartilage in joints, optionally by comparing a subject's joints before and after treatment with the composition.

The health of the liver can be assessed with imaging tools, such as X-rays, CT scans, ultrasounds (e.g., FibroScan), MRI, or Magnetic resonance spectroscopy (MRS), for detecting and measuring lesions, tumors, fat accumulation, fibrosis, abnormal blood flow, and liver stiffness. For example, a reduction in fat (lower echogenicity on ultrasound), decreased fibrosis/stiffness (lower kPa on FibroScan), improved texture, decreased liver size (if previously enlarged due to inflammation), and/or reduced ascites (fluid) shown on the imaging results indicate improvement in the health of the liver of a subject.

Imaging tools for inspecting and evaluating the health of endothelial glycocalyx are known. For example, the health of the endothelial glycocalyx can be assessed using suitable biosensor devices, such as those described in U.S. Pat. No. 8,759,095, the entirety of which is incorporated by reference herein. One suitable tool for assessing the health of endothelial glycocalyx is GLYCOCHECK® Microvascular Health Monitor available from Micro Vascular Health Solutions, which can be used to screen a subject's or patient's perfused boundary region (“PBR”) by measuring and monitoring changes in the PBR in real time. PBR is a measure for the depth of penetration of red blood cells in the glycocalyx (or into the region where healthy glycocalyx should be found). Low values of PBR, such as a value of less than 2.5 microns in vessels 5 to 20 microns in diameter, indicate a mechanically stable glycocalyx that protects the vessel wall against damage by circulating blood cells and other constituents, molecules or reagents circulating in the blood. Other measurable indicators include (blood) volume, width and dimension of the glycocalyx, vessel density, the number of perfused vessels per tissue surface versus total number of vessels, RBC filling percentage, capillary volume reserve, enzyme activity, and presence or absence of glycocalyx contributing constituents, nitric oxide concentration in the blood, etc. Changes in any of the foregoing parameters, alone or in combination, can be useful indicators for assessing glycocalyx health.

Alternatively and additionally, the improved health of blood vessels and joint health, optionally as a result of the promoted synthesis of GAGs and HA, reduced local and/or systematic inflammation, and/or improved mitochondrial function, may be indicated by the level(s) of one or more biological marker(s) in a biological sample of the subject compared to the level(s) of the same biological marker(s) in the same type of biological sample of the subject prior to administration of the composition.

Suitable biological samples for assessing the health of glycocalyx include, but are not limited to, whole blood, plasma, serum, and urine. Circulating markers for assessing the degradation of endothelial glycocalyx include, but are not limited to, Syndecan proteins (which tether GAGs and HA to the luminal surface of vascular endothelium) or fragments thereof, CD44 and its circulating extracellular domain (CD44 tether HA to endothelial cells), and circulating glycosaminoglycan and hyaluronic acid fragments. Several degradative enzymes that contribute to glycocalyx damage are known, such as Angiopoietin 2, Heparinase, Cathepsin B, Matrix metalloproteases (especially MMP-2, MMP-7 and MMP-9), and C-reactive protein (CRP). CRP is primarily produced by the liver as an index of systemic inflammatory status, but also directly damages endothelial glycocalyx. Elevated serum uric acid is also known to damage glycocalyx, and orotate can lower systemic uric acid concentrations. These markers primarily reflect factors that damage the glycocalyx. The beneficial biological activity of the disclosed compositions is not restricted to direct metabolic support of glycocalyx oligosaccharide synthesis, and includes anti-inflammatory activities that reduce glycocalyx shedding.

In some forms, the improved health of blood vessels and joint health, optionally as a result of the promoted synthesis of GAGs and HA, reduced local and/or systematic inflammation, and/or improved mitochondrial function, achieved by the disclosed composition is indicated by a change (e.g., an increase or decrease of at least 10%) in the level of one or more biological marker(s), such as any of those described above, in a subject, as indicated by the measurement of a biological sample (such as in the blood) of the subject following administration, such as oral administration of the composition to the subject.

In some forms, the improved health of blood vessels and joint health, optionally as a result of the promoted synthesis of GAGs and HA, reduced local and/or systematic inflammation, and/or improved mitochondrial function, achieved by the disclosed composition is indicated by the change (increase or decrease by at least 10%) in the level of one or more biological marker(s) in a subject, as indicated by the measurement of a biological sample (such as in the blood) of the subject compared to a control subject, as measured under the same conditions. A control subject is a subject or group of subjects that possess similar physiological conditions (e.g., same sex, similar age, and comparable pre-existing conditions, etc.) to the subject receiving the composition of glucosamine orotate salt and are administered a placebo composition. A placebo composition refers to a composition that contains all components of the composition of glucosamine orotate salt except for the glucosamine orotate salt and optionally in place of glucosamine orotate salt, contains a pharmacologically inactive substance with an appearance similar to that of glucosamine orotate salt, such as microcrystalline cellulose or lactose. The placebo composition is administered to the control subject or group of control subjects using the same administration regimen (e.g., dosage, frequency, time period) as the subject receiving the composition of glucosamine orotate salt. The measurement conditions (e.g., biological sample, imaging tool, pain assessment scale, time period, etc.) used to assess the biological markers and/or perform a visual inspection of the cell, tissue, or organ are also the same for the control subject or the group of control subjects as the subject receiving the composition of glucosamine orotate salt.

In some forms, the improved health of the liver, optionally as a result of hepatic protection against inflammatory damage, reduced hepatic inflammation and/or local hepatic injury, and/or improved mitochondrial function, achieved by the disclosed composition is indicated by body temperature and/or a change (e.g., an increase or decrease of at least 10%) in the level of one or more biological marker(s), such as liver transaminases (aspartate aminotransferase (AST), alanine aminotransferase (ALT), gamma-glutamyl transferase (GGT)), total Bilirubin and/or direct (conjugated) Bilirubin, one or more inflammatory cytokine(s), and/or other markers that reflect hepatocyte death or permeabilization and specifically indicate liver damage or protection, in a subject, as indicated by the measurement of a biological sample (such as in the blood) of the subject following administration, such as oral administration of the composition to the subject.

For example, the improved health of the liver, optionally as a result of hepatic protection against inflammatory damage, reduced hepatic inflammation and/or local hepatic injury, and/or improved mitochondrial function, achieved by the disclosed composition is indicated by a stable body temperature (e.g., body temperature fluctuation, such as body temperature drop, of less than 1%) of a subject following administration, such as oral administration of the composition to the subject.

For example, the improved health of the liver, optionally as a result of hepatic protection against inflammatory damage, reduced hepatic inflammation and/or local hepatic injury, and/or improved mitochondrial function, achieved by the disclosed composition is indicated by a change (e.g., an increase or decrease of at least 10%) in the level of one or more liver transaminases (aspartate aminotransferase (AST), alanine aminotransferase (ALT), and/or gamma-glutamyl transferase (GGT)), Total Bilirubin, and/or Direct (conjugated) Bilirubin, in a serum sample of a subject following administration, such as oral administration of the composition to the subject. For example, the glucosamine orotate salt in the composition is in an effective amount to lower the level of aspartate aminotransferase (AST) alanine aminotransferase (ALT), total Bilirubin and/or direct (conjugated) Bilirubin in a subject, as indicated by the measurement of a biological sample (such as in the blood) of the subject following administration, such as oral administration of the composition to the subject.

For example, the improved health of the liver, optionally as a result of hepatic protection against inflammatory damage, reduced hepatic inflammation and/or local hepatic injury, and/or improved mitochondrial function, achieved by the disclosed composition is indicated by reducing and/or ameliorating of one or more symptoms associated with a liver disease, such as iron overload, alcoholic liver disease, autoimmune hepatitis, and fatty liver disease (e.g., metabolism-associated fatty liver, steatohepatitis).

### III. Methods of Using Glucosamine Orotate Salt

Methods of using the glucosamine orotate salt in a subject in need thereof to promote synthesis of GAGs and HA, reduce local and/or systematic inflammation, and/or improve or maintain mitochondrial function, and thereby achieve one or more therapeutic goals, such as improving or maintaining the health of the endothelial glycocalyx of a subject; supporting the health of the blood vessels in a subject; supporting synthesis and/or maintenance of cartilage in joints and tendons in a subject; mitigating pain and/or joint damage due to osteoarthritis in a subject; reducing hepatic inflammation and/or injury in local hepatic and/or systemic inflammation, such as SIRS, SEPSIS, or multiple organ failure syndrome; protecting the liver against inflammatory damage; reducing and/or ameliorating one or more symptom(s) (e.g., chronic inflammation) associated with mitochondrial disease(s)/disorder(s); and/or reducing and/or ameliorating one or more symptom(s) (e.g., chronic inflammation) associated with disease(s)/disorder(s) related to cGAS-STING activation.

In some forms, the glucosamine orotate salt is administered for a sufficient period of time and in an effective amount to improve or maintain the health of the endothelial glycocalyx of a subject.

In some forms, the glucosamine orotate is administered for a sufficient period of time and in an effective amount to improve or maintain mitochondrial function of a subject.

In some forms, the glucosamine orotate salt is administered for a sufficient period of time and in an effective amount to support the health of the blood vessels in a subject.

In some forms, the method using the glucosamine orotate salt is administered for a sufficient period of time and in an effective amount to support synthesis and/or maintenance of cartilage in joints and tendons in a subject.

In some forms, the glucosamine orotate salt is administered for a sufficient period of time and in an effective amount to mitigate pain and/or joint damage due to osteoarthritis in a subject.

In some forms, the glucosamine orotate salt is administered for a sufficient period of time and in an effective amount to reduce hepatic inflammation and/or injury in local hepatic and/or systemic inflammation, such as SIRS, SEPSIS, or multiple organ failure syndrome.

In some forms, the glucosamine orotate salt is administered for a sufficient period of time and in an effective amount to protect the liver against inflammatory damage. For example, the glucosamine orotate salt is administered for a sufficient period of time and in an effective amount to protect otherwise normal animals (e.g., animal models in which the organism has not received a clinically-irrelevant hepatotoxic sensitizing agent like galactosamine or TCDD) from toxicity due to bacterial endotoxin (such as Salmonella Typhimurium endotoxin (LPS)) and other inflammatory stimuli which produce tissue damage through elicitation of endogenous inflammatory mediators.

In some other forms, the glucosamine orotate salt is administered for a sufficient period of time and in an effective amount to improve the appearance of skin (such as by reducing wrinkles and/or increasing the skin thickness) in a subject. In these forms, a topical composition containing glucosamine orotate salt can be applied topically to the skin. Reduced GAG and HA synthesis by fibroblasts in skin as a consequence of aging and photoaging results in thinning of skin, which enhances wrinkling. Application of the topical compositions can support GAG and HA synthesis locally in the skin and thereby reducing the appearance of wrinkles (e.g., filling in wrinkles secondary to skin thinning due to age or photodamage) and/or increasing the skin thickness. Methods for measuring reduction of wrinkles and skin thickness are known. Additionally, GAG and HA can be directly assessed by histology or through assays in the tissue (e.g., skin). Test kits and histology dyes for these measurements are known and commercially available.

In some other forms, the glucosamine orotate salt is administered for a sufficient period of time and in an effective amount to reduce and/or ameliorate one or more symptom(s) (e.g., chronic inflammation) associated with mitochondrial disease(s)/disorder(s) in a subject.

In some other forms, the glucosamine orotate salt is administered for a sufficient period of time and in an effective amount to reduce and/or ameliorate one or more symptom(s) (e.g., chronic inflammation) associated with disease(s)/disorder(s) associated with cGAS-STING activation in a subject.

Generally, the method of using the glucosamine orotate salt for any of the treatments described above includes (i) administering to the subject the composition containing glucosamine orotate salt, such as the nutraceutical composition, pharmaceutical composition, or cosmetic composition. The composition may be administered once daily, or repeated one or more times in a day. Optionally, the method further includes administering one or more additional active agent(s) before, during, or after administration of the composition containing glucosamine orotate salt.

The composition is administered via any suitable route. For example, a nutraceutical composition is administered orally; a pharmaceutical composition is administered orally or parenterally (e.g., intravenously, subcutaneously, or topically); and a cosmetic composition is administered orally, by injection, or topically, to a subject. The specific administration route is selected based on the specific therapeutic goal, composition type (e.g., nutraceutical, pharmaceutical, or cosmetic), and form of composition (solid, liquid, etc.).

For example, a pill, table, capsule, or liquid nutraceutical composition is orally administered for supporting the health of the blood vessels in a subject. For example, a tablet, capsule, solution, or suspension, nutraceutical or pharmaceutical composition is orally administered for alleviating osteoarthritis pain and/or repairing cartilage degradation. For example, a pill, table, capsule, or liquid nutraceutical or pharmaceutical composition is orally administered for mitigating pain and joint damage due to osteoarthritis in a subject and/or for reducing hepatic inflammation and injury in both local hepatic and systemic inflammations. For example, a liquid pharmaceutical composition is injected locally, for example, directly injected into joints or synovial fluid to support local cartilage maintenance or repair. For example, a liquid pharmaceutical composition is injected intravenously or subcutaneously for treatment of acute conditions, for example, vascular endothelial glycocalyx deterioration that occurs after trauma or during infections (including sepsis, bacterial infections, fungal infections, and viral infections, such as infections with SARS-CoV2). For example, a pill, table, capsule, or liquid nutraceutical or pharmaceutical composition is orally administered for protecting the liver against inflammatory damage or reducing hepatic inflammation and/or local hepatic injury in a subject. For example, a liquid pharmaceutical composition is injected intravenously or subcutaneously for protecting the liver against inflammatory damage or reducing hepatic inflammation and/or local hepatic injury in a subject. For example, a cosmetic composition, such as in the form of a gel, cream, ointment, or patch, is directly applied to the skin. For example, a liquid cosmetic composition is injected into the skin.

Optionally, the subject being treated using the methods disclosed herein has a disorder characterized by inadequate synthesis of glycosaminoglycans and hyaluronan (such as osteoarthritis) or a degraded endothelial glycocalyx. Optionally, the subject being treated has one or more symptom(s) characterized by impaired glycocalyx integrity or impaired blood-brain barrier function, such as neurological problems (e.g., cognitive decline, “brain fog”, and dementia). Glycocalyx integrity diminishes with aging, and consequent vascular endothelial problems contribute to numerous pathologies associated with aging, including peripheral artery disease, effects of weight loss (with or without use of weight-reduction drugs such as GLP-1 agonists), or sarcopenia. Glycocalyx structure and function are also compromised in both Type 1 and Type 2 diabetes. Optionally, the subject being treated has a disease or disorder characterized by glycocalyx shedding, such as acute trauma, systemic inflammation, or infections, such as SARS-CoV2 (COVID) infections.

Optionally, the subject being treated has experienced or is experiencing one or more symptom(s) associated with a disease or disorder characterized by mitochondrial dysfunction or damage (also referred to herein as “mitochondrial disease or disorder”), such as those involving mitochondrial dysfunction in their pathogenesis. Clinical symptoms of mitochondrial diseases affecting oxidative phosphorylation by impairing the electron transport chain are generally attributable to impaired mitochondrial reserve energy capacity (ability to respond to fluctuations in cellular energy demands), oxidative damage due to spillover of oxygen radicals from the electron transport chain, or inflammation due to mitochondrial fragility and osmotic swelling or shrinkage of intramitochondrial compartments. In defective mitochondria, or in situations where cellular energy demands exceed mitochondrial energy production capacity, consequent mitochondrial disruption results in release of mitochondrial DNA is released into the cytosol, which leads to activation of pro-inflammatory pathways (e.g., cGAS-STING). Examples of mitochondrial diseases or disorders include, but are not limited to, congenital mitochondrial cytopathy, neurodevelopmental delay, and age-related neurodegenerative disease. Augmentation of uridine nucleotides with glucosamine orotate can reduce likelihood of mitochondrial disruption and permeabilization, thereby improving reserve energy capacity and reducing mitochondrial DNA leakage into the cytosol or circulation. Increased intracellular and specifically intramitochondrial uridine nucleotides can protect mitochondrial structure and function by activating the mitochondrial ATP-sensitive potassium channel, which compensates for osmotic deformation of mitochondrial compartments related to proton pumping across the mitochondrial inner membrane during mitochondrial respiration and oxidative phosphorylation.

Other examples of mitochondrial disorders include LHON (Lebers hereditary optic neuropathy) “Mitochondrial blindness”, KSS (Kearns-Sayre Syndrome), PMPS (Pearson Marrow-Pancreas Syndrome), CPEO (Chronic progressive external opthalmoplegia), Multiple mtDNA deletion syndrome, MtDNA depletion syndrome, Complex I deficiency, Complex II (SDH) deficiency, Complex III deficiency, Cytochrome c oxidase (COX, Complex TV) deficiency, Complex V deficiency, Adenine Nucleotide Translocator (ANT) deficiency, Pyruvate dehydrogenase (PDH) deficiency, Ethylmalonic aciduria with lactic acidemia, 3-Methyl glutaconic aciduria with lactic acidemia, materially inherited thrombocytopenia and leukemia syndrome, MARIAHS syndrome (Mitrochondrial ataxia, recurrent infections, aphasia, hypouricemia/hypomyelination, seizures, and dicarboxylic aciduria), Cyclic vomiting syndrome with declines during infection, 3-Hydroxy isobutyric aciduria with lactic acidemia, Diabetes mellitus with lactic acidemia, Aminoglycoside-associated deafness, Dilated cardiomyopathy, Splenic Lymphoma, Wolfram syndrome, Multiple mitochondrial DNA deletion syndromes, and Renal Tubular Acidosis/Diabetes/Ataxis syndrome. Mitochondrial diseases have complex and heterogeneous phenotypes, but at the cellular level, they are defined by genetic defects in mitochondrial oxidative phosphorylation. Mutations or deletions in approximately 300 different genes have been reported to result in clinical mitochondrial disease symptoms. Diagnosis typically involves whole exome sequencing of both nuclear and mitochondrial DNA, combined with clinical phenotypes or symptom clusters

In some forms, the subject being treated with the composition has experienced or is experiencing one or more symptom(s) associated with one or more mitochondrial diseases and/or disorders (e.g., diseases and/or disorders that involve mitochondrial dysfunction in their pathogenesis) including, but not limited to, Alzheimer's dementia, Lewy body dementia, Huntington's disease, Amyotrophic lateral sclerosis, Parkinson's disease, Hepatorenal syndrome, Acute liver failure, NASH (non-alcoholic steatohepatitis), Diabetes mellitus (particularly Type II), reperfusion injury in acute myocardial infarctions, Eclampsia/pre-eclampsia, Oopause infertility, and Ischemic heart disease/Angina.

Common symptoms of mitochondrial diseases which may be present alone or in conjunction with the mitochondrial diseases and disorders described above include chronic inflammation, cardiomyopathy, muscle weakness and atrophy, ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

In some forms, the subject being treated has experienced or is experiencing one or more symptom(s) associated with a disease or disorder associated with cGAS-STING activation, such as trauma, infection, or a disease/disorder that is at least partially a pathophysiological consequence of cGAS-STING activation. Unregulated or overactivation of the cGAS-STING pathway can lead to both systemic and localized inflammation, which can be detrimental to tissue health (e.g., liver tissue health) if this activation is not dampened. A pathophysiological consequence of cGAS-STING activation can be cytokine storms, also known as cytokine release syndrome, which is a disorder that is characterized by excessive pro-inflammatory signaling proteins (e.g., cytokines) in the subject's blood or a tissue. Cytokine storms can be triggered by severe trauma, infection, or cancer therapies and can result in multiple organ failure, also known as sequential organ failure. This often begins with inflammatory liver injury and progresses towards the failure of other tissues, including kidney and lungs. Cytokine storms can also manifest as systemic inflammatory response syndrome (SIRS) after severe trauma or various infections, including coronaviruses. Cytokine storms are also a known adverse effect of some cancer chemotherapies and cancer immunotherapies, such as administration of CAR-T cells that deplete B lymphocytes for treating lymphomas. Other examples of diseases or disorders that are pathophysiological consequences of cGAS-STING activation include, but are not limited to, sequential organ failure syndrome, sepsis, neuroinflammation, chronic inflammation associated with aging, vascular inflammation (e.g., atherosclerosis), Type 2 diabetes, fatty liver, and inflammatory bowel disease.

Further, hepatic cGAS-STING activation mediates or exacerbates both acute liver injuries and chronic liver diseases. Examples of acute liver injuries mediated or exacerbated by cGAS-STING actions include, but are not limited to, systemic bacterial or viral sepsis, liver inflammation driven by severe trauma (which can be extrahepatic injury), radiation injury, chemical damage (e.g. carbon tetrachloride), acetaminophen toxicity, surgical stress during liver resection or transplant, and liver ischemia-reperfusion injury or blood loss (hypovolemic shock). Examples of chronic liver diseases injuries mediated or exacerbated by cGAS-STING actions include, but are not limited to, iron overload, alcoholic liver disease, autoimmune hepatitis, and fatty liver disease (e.g., metabolism-associated fatty liver, steatohepatitis).

Optionally, the subject being treated using the methods disclosed herein has osteoarthritis and its associated joint pain. Compared to standard treatments for osteoarthritis using nonsteroidal anti-inflammatory drugs (NSAIDs) and glucosamine sulfate, the methods using glucosamine orotate salt offer several advantages. For example, although NSAIDs can reduce pain, they have long term adverse effects that can require cessation or dose reduction, notably diminishing thickness and integrity of the lining of the intestinal tract. NSAIDs also do not promote active cartilage maintenance or repair. Glucosamine sulfate is used for treatment of osteoarthritis pain, with only moderate efficacy in clinical trials. The plasma concentrations of glucosamine after oral administration of glucosamine sulfate at the standard recommended dose of 1500 mg per day as a single dose reach a peak concentration of approximately 10 micromolar, which is below the threshold for elevating glycosaminoglycan synthesis in chondrocytes in vitro, indicating that clinical efficacy primarily attributable to anti-inflammatory effects. Further, as discussed above, glucosamine sulfate includes both sulfate and 2 molar equivalents of sodium chloride (NaCl). The molecular weight of crystalline glucosamine sulfate+2NaCl is 573.3 g/mol, whereas the molecular weight of glucosamine base is 179, only 35% of the total MW for glucosamine sulfate. Dose escalation of glucosamine sulfate to attempt to enhance cartilage synthesis by precursor loading may be limited by the inorganic salt load co-delivered with the glucosamine, which can be a concern for exacerbation of hypertension with NaCl, or for osmotic fluid retention in the intestines with sulfate. In contrast, glucosamine orotate salt used in the disclosed methods contains no inorganic counter ions, and therefore is not subject to such limitations on dose escalation. Further, without being bound to any theories, orotate elevates intracellular uridine nucleotides, providing the essential cofactor that must accompany glucosamine for boosting intracellular concentrations of UDP-sugars, the immediate precursors for glycosaminoglycan synthesis. Additionally, orotate is uricosuric, promoting clearance of uric acid from the circulation by blocking its reuptake from renal tubules after initial excretion via glomeruli in the kidney. Elevated serum uric acid is an established risk factor contributing to severity of osteoarthritis pain, inflammation and cartilage degradation. Thus, glucosamine orotate salt provides superior efficacy versus glucosamine sulfate.

Optionally, the subject being treated has a disease or disorder in which hepatoxicity due to endotoxin or other inflammatory stimuli or mediators are implicated in their etiology (whether or not systemic sepsis syndrome is present). Diseases and disorders in which damage to the liver by endotoxin or its mediators (e.g. TNF) are implicated include, but are not limited to, Reye's Syndrome, Alcoholic Liver Damage, Fulminant Hepatitis, Viral Hepatitis, Parasitic Infections, Hepatic damage during total parenteral nutrition Hepatic complications, Lead Poisoning, Partial Hepatectomy, Postanesthetic Hepatitis, Cholestatic Hepatitis, Liver Transplantation, etc. Optionally, the subject being treated is at risk of being exposed to a bacterial endotoxin (LPS) derived from gram negative bacteria, such as Salmonella typhimurium or E. coli.

Optionally, the method further includes administering one or more additional active agent(s) to the subject that are used with glucosamine orotate salt as a combination therapy, such as to further reduce inflammation. For example, one or more active agent(s) that modulate monocyte cytokine release can be administered to the subject prior to, simultaneously with, and/or after administration of the glucosamine orotate salt or composition thereof. Examples of active agents that modulate monocyte cytokine release include any suitable anti-inflammatory or immunosuppressant agents which include, but are not limited to, pharmacological, natural, and biological agents that inhibit pro-inflammatory pathways, such as corticosteroids (e.g., dexamethasone), antibodies (e.g., adalimumab, sarilumab), cytokine inhibitors (e.g., anakinra, tocilizumab), natural polyphenols (e.g., curcumin, resveratrol), phosphodiesterase 4 inhibitors (e.g., apremilast, roflumilast), JAK/STAT inhibitors (e.g., upadacitinib, tofacitinib), and omega-3 fatty acids (e.g., EPA, DHA).

The composition can be administered for any suitable time period as desired. In some forms, following daily administration of the composition for a suitable time period, an effective amount of the glucosamine orotate salt to achieve a desired therapeutic benefit is delivered to the subject. In other words, following daily administration of the composition for a suitable time period, the amount of glucosamine orotate salt administered to the subject is effective for one or more therapeutic benefit(s) occur(s) and be observed. Generally, following daily administration of the composition for 1 to 8 weeks or 1 to 4 weeks or weekly administration for a time period ranging from 2 to 8 weeks or from 2 to 4 weeks, the desired therapeutic benefit occurs and can be observed using suitable means, such as those described above in the “Dosages and concentrations” section (e.g., imaging and biomarkers). The specific time period for a desired therapeutic benefit to occur depends on the specific health benefit, the dose of actives being administered, the administration route, the health condition of the subject, etc. For example, for acute conditions with extensive glycocalyx shedding, such as trauma, systemic inflammation, or viral infections, a period of treatment for 1 to 4 weeks may help reduce severity of such conditions and support recovery from the health crisis (where 30-day mortality is often a marker for clinical success and likelihood of recovery). For counteracting age-related pathologies (such as supporting joint health and/or the health of endothelial glycocalyx), therapeutic benefits will likely be apparent within a few weeks (e.g., within 8 weeks, within 6 weeks, or within 4 weeks), and can be sustained during continued treatment.

In some forms, the therapeutic benefits of the composition, such as supporting the joint health, the health of endothelial glycocalyx, and/or the health of the liver, can be indicated by visual inspection of cartilage, endothelial glycocalyx, and/or liver of the subject using an imaging tool. For example, the joint health, health of endothelial glycocalyx, and/or health of the liver is indicated by visual inspection of cartilage, endothelial glycocalyx, the liver of the subject using an imaging tool compared to the appearance of the cartilage, endothelial glycocalyx, and/or the liver prior to administering the composition using the same imaging tool. For example, the joint health, health of endothelial glycocalyx, and/or the health of the liver is indicated by visual inspection of cartilage, endothelial glycocalyx, and/or the liver of the subject using an imaging tool compared to the appearance of the cartilage, endothelial glycocalyx, and/or the liver following administration, such as oral administration to a control subject, as measured under the same conditions.

As discussed above, imaging tools suitable for assessing the health of cartilage and related factors in joint health include, but are not limited to X-rays, CT Scans, and MRI, which can quantify the thickness and density of cartilage in joints. Imaging tools for inspecting and evaluating the health of endothelial glycocalyx are known and described above in the “Dosages and concentrations” section, for example, biosensor devices, such as those described in U.S. Pat. No. 8,759,095 and GLYCOCHECK® Microvascular Health Monitor available from Micro Vascular Health Solutions. Imaging tools suitable for assessing the health of the liver include, but are not limited to, X-rays, CT scans, ultrasounds (e.g., FibroScan), MRI, and MRS, which can be used to detect lesions, tumors, fat accumulation, and fibrosis.

In some forms, the therapeutic benefits of the composition, such as supporting joint health, can be indicated by the health of cartilage assessed using a joint fluid sample, by inspecting its appearance, color, and/or viscosity; and/or by the level of glucose, inflammation-related proteins, uric acid, and/or lactate dehydrogenase.

In some forms, the therapeutic benefits of orally administered composition, such as supporting joint health, supporting the health of endothelial glycocalyx, and/or supporting the health of liver, can be indicated by the level of one or more biological markers in a biological sample from the subject. Suitable biological samples and biological markers for assessing the joint health and health of endothelial glycocalyx are described above in the “Dosages and concentrations” section. For example, the support of joint health, health of endothelial glycocalyx, and/or health of liver in a subject is indicated by a change (e.g., an increase or decrease of at least 10%) in the level of one or more biological marker(s), such as any of those described above, in a subject, as indicated by the measurement of a biological sample (such as in the blood) of the subject following administration, such as oral administration of the composition to the subject. For example, the support of joint health, health of endothelial glycocalyx, and/or health of liver in a subject is indicated by the change (increase or decrease by at least 10%) in the level of one or more biological marker(s) in a subject, as indicated by the measurement of a biological sample (such as in the blood) of the subject compared to the oral administration to a control subject, as measured under the same conditions.

In some forms, the therapeutic benefits of the composition, such as mitigating pain and joint damage due to osteoarthritis in a subject, can be indicated by reduced pain level in the subject. Pain assessment scales allow quantitative measurement of a subject's pain and its changes over time are useful for assessing therapeutic benefits in subjects with osteoarthritis in response to ingestion of the compositions containing glucosamine orotate salt. For example, the pain level associated with osteoarthritis in the subject as measured using a suitable pain assessment scale, such as the Numerical Pain Rating Scale (NPRS), is reduced compared to the pain level associated with the osteoarthritis in the subject prior to administering the composition as measured using the same pain assessment scale. For example, the pain level associated with osteoarthritis in the subject as measured using a suitable pain assessment scale, such as the Numerical Pain Rating Scale (NPRS), is lower compared to the pain level associated with the osteoarthritis in the subject following administration, such as oral administration to a control subject, as measured using the same pain assessment scale.

In some forms, the therapeutic benefits of the composition, such as reducing hepatic inflammation and/or injury in local hepatic and/or systemic inflammations, such as SIRS, SEPSIS, or multiple organ failure syndrome in a subject, can be indicated by one or more inflammatory biomarkers and/or 30-day mortality. For example, the reduced hepatic inflammation and/or injury in local hepatic and/or systemic inflammations in a subject is indicated by a change (e.g., an increase or decrease of at least 10%) in the level of one or more inflammatory biomarker(s) in a subject, as indicated by the measurement of a biological sample (such as in the blood) of the subject following administration, such as oral administration of the composition to the subject. For example, the reduced hepatic inflammation and/or injury in local hepatic and/or systemic inflammations in a subject is indicated by the change (increase or decrease by at least 10%) in the level of one or more inflammatory biomarker(s) in a subject, as indicated by the measurement of a biological sample (such as in the blood) of the subject compared to a control subject, as measured under the same conditions.

In some forms, the support of the health of the liver is indicated by, over a 30-day, 60-day, or 90-day time period maintaining the level of each of one or more biological marker(s) in the subject within a range from a value that is 50% below the lower limit of a normal range to a value that is 50% above the upper limit of a normal range of the respective biological biomarker following administration, such as oral administration of the glucosamine orotate salt or the composition to the subject once or twice daily.

In some forms, the support of the health of the liver is indicated by, over a 30-day, 60-day, or 90-day time period maintaining the level of each of one or more biological marker(s) in the subject within the normal range of the respective biological biomarker following administration, such as oral administration of the glucosamine orotate salt or the composition to the subject once or twice daily.

For example, administration of the composition results in maintaining levels of AST in a range from 4 U/L to 72 U/L, maintaining levels of ALT in a range from 3.5 U/L to 82.5 U/L; maintaining levels of total Bilirubin in a range from 0.0 mg/dL to 1.8 mg/dL; and/or maintaining levels of direct Bilirubin in a range from 0.0 mg/dL to 0.45 mg/dL. For example, administration of the composition results in maintaining levels of AST in a range from 8 U/L to 48 U/L, maintaining levels of ALT in a range from 7 U/L to 55 U/L; maintaining levels of total Bilirubin in a range from 0.0 mg/dL to 1.2 mg/dL; and/or maintaining levels of direct Bilirubin in a range from 0.0 mg/dL to 0.3 mg/dL.

In some forms, the support of the health of the liver is indicated by reduce and/or ameliorate one or more symptoms associated with a liver disease, such as iron overload, alcoholic liver disease, autoimmune hepatitis, and fatty liver disease (e.g., metabolism-associated fatty liver, steatohepatitis).

In some forms, the therapeutic benefits of the composition, such as protecting the liver against inflammatory damage (such as in Metabolic Dysfunction-Associated Steatohepatitis, fatty liver disease, bacterial endotoxin, etc.) in a subject, can be indicated by biomarkers, imaging results, and/or body temperature. Suitable biomarkers for assessing liver health include, but are not limited to, liver transaminases, such as aspartate aminotransferase (AST), alanine aminotransferase (ALT), gamma-glutamyl transferase (GGT), total Bilirubin, and/or direct (conjugated) Bilirubin, inflammatory cytokine(s); and/or other markers that reflect hepatocyte death or permeabilization and specifically indicate liver damage or protection. For example, the liver protection effect in a subject is indicated by a change (e.g., an increase or decrease of at least 10%) in the level of one or more liver transaminase(s) (such as AST and/or ALT), Total Bilirubin, and/or Direct Bilirubin in a serum sample of the subject following administration, such as oral administration of the composition to the subject. For example, the liver protection effect in a subject is indicated by a restoration toward a normal level of one or more liver transaminase(s) in a subject, as indicated by the measurement of a biological sample (such as in the blood) of the subject following administration, such as oral administration of the composition to the subject.

In some forms, protection of the liver is indicated by (a) a stable body temperature (e.g., body temperature fluctuation, such as body temperature drop, of less than 1%); (b) a level of aspartate aminotransferase (AST) below about 96 U/L and/or a level of alanine aminotransferase (ALT) below about 110 U/L in a subject, as indicated by the measurement of a biological sample (such as in the blood) of the subject; and/or (c) a normal liver imaging result of the subject using an imaging tool, following administration, such as oral administration of the composition to the subject once or twice daily over a time period of 30 days, 60 days, or 90 days.

In some forms, the reduction of the inflammation in the liver or a health benefit is indicated by (a) a lowered level of aspartate aminotransferase (AST) and/or alanine aminotransferase (ALT) in a subject, as indicated by the measurement of a biological sample (such as in the blood) of the subject compared to the AST and/or ALT level in the subject prior to administering the composition; and/or (b) visual inspection of a liver imaging result of the subject using an imaging tool compared to the result of the subject prior to administering the composition, following administration, such as oral administration of the composition to the subject.

In some forms, administration of the composition results in the lowering of abnormally high alanine aminotransferase (ALT) and/or aspartate aminotransferase (AST) in the subject toward the normal range of ALT and/or AST. For example, administration, such as oral administration, of the composition once or twice daily results in a general trend of lowering ALT and/or AST levels in a subject toward the normal range of ALT and/or AST over a time period of 30 days, 60 days, or 90 days. In some forms, administration of the composition results in lowering AST levels in a subject from above 96 U/L to ≤96 U/L (e.g., below about 100 U/L, 90 U/L, 80 U/L, 70 U/L, 60 U/L, 50 U/L, 40 U/L, 30 U/L, 20 U/L, 10 U/L or 4 U/L, such as below 48 U/L). In some forms, administration of the composition results in lowering ALT from above 110 U/L to ≤110 U/L (e.g., below about 100 U/L, 90 U/L, 80 U/L, 70 U/L, 60 U/L, 50 U/L, 40 U/L, 30 U/L, 20 U/L, 10 U/L, or 3 U/L, such as below 55 U/L). In some forms, administration of the composition results in improvement of one or more tissue characteristics from the imaging results, such as reduction in fat (lower echogenicity on ultrasound), decreased fibrosis/stiffness (lower kPa on FibroScan), improved texture, decreased liver size (if previously enlarged due to inflammation), and/or reduced ascites (fluid).

In some forms, the therapeutic benefits of the composition, such as reducing and/or ameliorating one or more symptom(s) (e.g., chronic inflammation) associated with a mitochondrial disease or disorder, are indicated by (a) a lowered level of aspartate aminotransferase (AST) and/or alanine aminotransferase (ALT) in a subject, as indicated by the measurement of a biological sample (such as in the blood) of the subject compared to the AST and/or ALT level in the subject prior to administering the composition; and/or (b) visual inspection of the imaging result of a target organ of the subject using an imaging tool compared to the result of the subject prior to administering the composition, following administration, such as oral administration of the composition to the subject.

In some forms, administration of the composition results in lowering AST levels in a subject to ≤96 U/L (e.g., below about 100 U/L, 90 U/L, 80 U/L, 70 U/L, 60 U/L, 50 U/L, 40 U/L, 30 U/L, 20 U/L, 10 U/L or 4 U/L, such as below 48 U/L). In some forms, administration of the composition results in lowering ALT≤110 U/L (e.g., below about 100 U/L, 90 U/L, 80 U/L, 70 U/L, 60 U/L, 50 U/L, 40 U/L, 30 U/L, 20 U/L, 10 U/L, or 3 U/L, such as below 55 U/L).

In some forms, the therapeutic benefits of the composition, such as reducing and/or ameliorating one or more symptom(s) (e.g., chronic inflammation) associated with a disease or disorder associated with cGAS-STING activation, are indicated by (a) a lowered level of aspartate aminotransferase (AST) and/or alanine aminotransferase (ALT) in a subject, as indicated by the measurement of a biological sample (such as in the blood) of the subject compared to the AST and/or ALT level in the subject prior to administering the composition; and/or (b) visual inspection of the imaging result of a target organ of the subject using an imaging tool compared to the result of the subject prior to administering the composition, following administration, such as oral administration of the composition to the subject.

Generally, the glucosamine orotate salt in the composition is delivered to the subject at a daily dose ranging from about 1 g to about 8 g, such as from about 2 g to about 8 g, or from about 4 g to about 6 g. The daily dose delivered to the subject can be in a single dosage form or divided into two or more doses per day, such as two or three doses per day.

For example, the nutraceutical composition is delivered to a subject at a daily dose ranging from about 2 g to about 8 g of glucosamine orotate salt, where the subject has a disease or disorder characterized by inadequate synthesis of glycosaminoglycans and hyaluronan (such as osteoarthritis) or a degraded endothelial glycocalyx.

Without being bound to any theories, it is believed that the therapeutic benefits achieved by glucosamine orotate salt are achieved via various mechanisms. Although metabolic support for glycocalyx synthesis by precursor loading is a fundamental feature of glucosamine orotate salt, additional mechanisms of action can contribute. For example, orotate has uricosuric properties, enhancing excretion of uric acid via the urine by blocking uric acid reuptake after its filtration into the renal tubules. Orotic acid is an inhibitor of URAT1, the primary transporter involved in uric acid reuptake, and URAT1 inhibitors in general lower serum uric acid. Additionally, de novo synthesis of purines, from which uric acid is derived as a degradation product, requires PRPP as a ribose phosphate donor. By competing with purine metabolites for PRPP, orotate can reduce de novo purine synthesis and thereby further lower serum uric acid beyond the effect on URAT1 in renal tubules.

Further, without being bound to any theory, another mechanism of action in some tissues is a consequence of elevated intracellular uridine nucleotides with orotate, including and especially in the form of glucosamine orotate salt. Energy stress in endothelial cells, whether due to impaired mitochondrial function or excessive energy demand on cells, can result in permeabilization of the mitochondrial inner and outer membranes, in part due to osmotic shrinkage and swelling of mitochondrial compartments, specifically the intermembrane space between the inner and outer membranes, and the matrix, the inner space where mitochondrial DNA and many enzymes involved in energy metabolism are located. Energy stress or other forms of mitochondrial damage can result in leakage of mitochondrial DNA into the cytosol. In the cytosol, there is a system for detecting and responding to ectopic double-stranded DNA, whether from viruses, nuclear DNA damage, endocytosis of bacteria, or, with greatest relevance to glycocalyx integrity, mitochondrial DNA. This system involves cGAS (cyclic Guanosine monophosphate-Adenosine monophosphate synthase), which produces cGA (cyclic Guanosine monophosphate-Adenosine monophosphate) in response to contact with double-stranded DNA. cGAS in turn activated STING (STimulator of INterferon Genes), resulting in activation of interferon signaling and NRP3 inflammasome activation. Inflammation in endothelial cells can result in glycocalyx shedding. Elevation of intracellular uridine nucleotides, in this case using orotate as a precursor, results in elevation of uridine nucleotides within the mitochondrial intermembrane space. Uridine diphosphate (UDP) is a direct activating ligand of the mitochondrial ATP-sensitive potassium channel (mito-KATP), which regulates mitochondrial architecture by counteracting osmotic changes due to proton pumping via the mitochondrial electron transport chain, by regulating potassium entry into the mitochondrial matrix. This protective effect on mitochondrial architecture by elevated uridine nucleotides reduces the risk of mitochondrial permeabilization during energy stress and in other pathological conditions, in turn reducing mitochondrial DNA leakage into the cytosol and consequent activation of cGAS-STING pro-inflammatory signaling. Through this and other mechanisms, elevation of intracellular uridine nucleotides in the endothelial cells and other tissues including the liver, can dampen inflammatory processes that damage the endothelial glycocalyx, including expression and secretion into the circulation of markers and mediators listed above, which can be quantified with immunoassays or other standard methods.

For example, osteoarthritis and its associated joint pain are related to a shift in the balance of cartilage degradation and repair during its normal turnover toward net loss of cartilage, as chondrocytes and synoviocytes undergo age-related decline in cell numbers and functional capacity, combined with “wear and tear” in weight-bearing joints over time. Cartilage contains glycosaminoglycans and proteoglycans (for which glucosamine and uridine nucleotides are essential precursors), along with collagen and elastin. Inflammation generally accompanies cartilage deterioration in osteoarthritis and contributes to pain. In osteoarthritis, cartilage between bones in joints becomes thinner and less resilient. Eventually, the cartilage cushion between bones breaks down to the point where joint replacement may be necessary to alleviate pain and improve functional capacity. Medical treatment for osteoarthritis should alleviate pain by dampening inflammation, not just interfering with pain perception. Furthermore, medical treatment should slow the rate of cartilage degradation and actively promote cartilage maintenance and repair. It is believed that the compositions disclosed herein have anti-inflammatory activity and also directly support cartilage synthesis by providing immediate precursors for synthesis of glycosaminoglycans, which are important structural components of cartilage.

Additionally, without being bound to any theory, in addition to serving as a precursor for glycosaminoglycans, glucosamine can also act in signaling pathways affecting several aspects of cell function. In addition to the familiar posttranslational phosphorylation of proteins in signaling cascades, N-acetylglucosamine can undergo via reversable attachment to serine or threonine residues on proteins (“O-GlcNAc” groups) by the enzyme O-GlcNAc transferase with UDP-N-acetylglucosamine as the donor molecule. This process is known as “O-GlcNAcylation”, and the enzyme β-N-acetylglucosaminidase (O-GlcNAcase) can remove the O-GlcNac groups from proteins. Glucosamine is metabolized to form intracellular N-acetylglucosamine, which is not efficiently taken up into cells from extracellular fluids, and intracellular concentrations of UDP-GlcNAc can be regulated by availability of both UTP (which can be augmented with exogenous orotate) and GlcNac. The rate and extent of O-GlcNAcylation of regulatory proteins can in turn be regulated by UDP-GLcNAc availability. Thus, the glucosamine orotate salt provides both precursors for O-GlcNAc modulation of protein functions. GlcNAcylated proteins are known to regulate transcription, ubiquitination, cell cycling, cell stress, and nutrient utilization. Acute increases in protein O-GlcNAcylation are generally cytoprotective.

The disclosed glucosamine orotate salt, compositions thereof, methods of making thereof, and methods of using thereof can be further understood through the following enumerated paragraphs.
Paragraph 1. A glucosamine orotate salt.
Paragraph 2. A glucosamine orotate salt produced by a process comprising:
   (i) passing glucosamine hydrochloride through a column and into a suspension of orotic acid to form the glucosamine orotate salt,
   wherein the column comprises an ion exchange resin, and
   optionally wherein the suspension of orotate acid is under stirring during step (i).
Paragraph 3. The glucosamine orotate of paragraph 2, wherein the molar ratio of glucosamine hydrochloride to orotic acid is about 1:1.
Paragraph 4. The glucosamine orotate salt of paragraph 2 or 3, wherein step (i) comprises:
   (a) loading an aqueous solution of glucosamine hydrochloride onto the column, and
   (b) washing the column with deionized water.
Paragraph 5. The glucosamine orotate salt of any one of paragraphs 2-4, wherein the process further comprises, prior to step (i): (c) washing the ion exchange resin, and (d) loading the washed ion exchange resin into the column.
Paragraph 6. The glucosamine orotate salt of any one of paragraphs 2-5, wherein the process further comprises, prior to step (i): (e) mixing orotic acid in an organic solvent, optionally the mixing is performed by stirring.
Paragraph 7. The glucosamine orotate salt of paragraph 6, wherein the organic solvent is methanol.
Paragraph 8. The glucosamine orotate salt of any one of paragraphs 2-6, wherein the process further comprises, after step (i): (f) isolating the glucosamine orotate salt, optionally the isolation is performed by evaporation of the deionized water and organic solvent under vacuum.
Paragraph 9. The glucosamine orotate salt of any one of paragraphs 1-8, represented by the following formula:

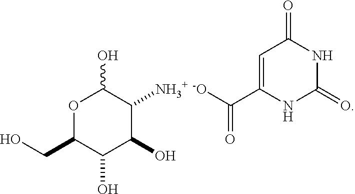

Paragraph 10. The glucosamine orotate salt of any one of paragraphs 1-8, in a tetrahydrate crystal form.
Paragraph 11. The glucosamine orotate salt of paragraph 10, represented by Glucosamine Orotate·4H₂O.
Paragraph 12. A method for producing glucosamine orotate salt, comprising:
   (i) passing glucosamine hydrochloride through a column and into a suspension of orotic acid to form the glucosamine orotate salt,
   wherein the column comprises an ion exchange resin, and
   optionally wherein the suspension of orotate acid is under stirring during step (i).
Paragraph 13. The method of paragraph 12, wherein the molar ratio of glucosamine hydrochloride to orotic acid is about 1:1.
Paragraph 14. The method of paragraph 12 or 13, wherein step (i) comprises:
   (a) loading an aqueous solution of glucosamine hydrochloride onto the column, and
   (b) washing the column with deionized water.
Paragraph 15. The method of any one of paragraphs 12-14, wherein the process further comprises, prior to step (i): (c) washing the ion exchange resin, and (d) loading the washed ion exchange resin into the column.
Paragraph 16. The method of any one of paragraphs 12-15, wherein the process further comprises, prior to step (i): (e) mixing orotic acid in an organic solvent, optionally the mixing is performed by stirring.
Paragraph 17. The method of paragraph 16, wherein the organic solvent is methanol.
Paragraph 18. The method of any one of paragraphs 12-17, wherein the process further comprises, after step (i): (f) isolating the glucosamine orotate salt, optionally the isolation is performed by evaporation of the deionized water and organic solvent under vacuum.
Paragraph 19. The method of any one of paragraphs 12-18, wherein the glucosamine orotate salt is represented by the following formula:

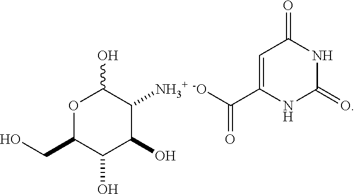

Paragraph 20. A composition comprising:
   (a) the glucosamine orotate salt of any one of paragraphs 1-11; and
   optionally, (b) one or more non-ionic excipients and/or one or more carriers.
Paragraph 21. The composition of paragraph 20, wherein the glucosamine orotate salt is present in an effective amount to improve or maintain the health of the endothelial glycocalyx of a subject.
Paragraph 22. The composition of paragraph 20 or 21, wherein the glucosamine orotate salt is present in an effective amount to restore, improve, or support the health of blood vessels, and/or to improve maintenance or repair of cartilage in joints or tendons in a subject.
Paragraph 23. The composition of any one of paragraphs 20-22, wherein the glucosamine orotate salt is present in an effective amount to restore, improve, or support the health of liver.
Paragraph 24. The composition of paragraph 20 or 21, wherein the glucosamine orotate salt is present in an effective amount to support glycosaminoglycans and hyaluronan synthesis locally in the skin of a subject.
Paragraph 25. The composition of any one of paragraphs 20-24, wherein glucosamine orotate salt is present in an effective amount to promote synthesis of glycosaminoglycans and hyaluronan.
Paragraph 26. The composition of any one of paragraphs 20-25, further comprising (1) diosmin and/or hesperidin; (2) citrulline and/or arginine; or a combination of (1) and (2).
Paragraph 27. The composition of any one of paragraphs 20-26, in a liquid form or solid form.
Paragraph 28. The composition of any one of paragraphs 20-27, in the form of dry powder, a tablet, a soft or hard chew, a gummy, a pill, or a capsule.
Paragraph 29. The composition of paragraph 27 or 28, wherein when in solid form, the one or more excipients comprises a desiccant.
Paragraph 30. The composition of any one of paragraphs 20-29, in a dosage form for administration to a subject, wherein the glucosamine orotate salt is present in the dosage form in an amount ranging from about 1 g to about 8 g, from about 2 g to about 8 g, or from about 4 g to about 6 g.
Paragraph 31. A method for providing a health benefit to a subject in need thereof, wherein the health benefit is improving or maintaining the health of the endothelial glycocalyx of the subject comprising:
   (i) administering the composition of any one of paragraphs 20-22 and 25-30 to the subject.
Paragraph 32. A method for providing a health benefit to a subject in need thereof, wherein the health benefit is supporting the health of the blood vessels and/or supporting the synthesis and/or maintenance of cartilage in joints and tendons in the subject, comprising:
   (i) administering the composition of any one of paragraphs 20-22 and 25-30 to the subject.
Paragraph 33. A method for providing a health benefit to a subject in need thereof, wherein the health benefit is mitigating pain and joint damage due to osteoarthritis in the subject, comprising:
   (i) administering the composition of any one of paragraphs 20-22 and 25-30 to the subject.
Paragraph 34. A method for supporting the health of the liver in a subject, comprising:
   (i) administering the composition of any one of paragraphs 20, 23, and 25-30 to the subject.
Paragraph 35. A method for protecting the liver against inflammatory damage or reducing inflammation in the liver in a subject, comprising:
   (i) administering the composition of any one of paragraphs 20, 23, and 25-30 to the subject.
Paragraph 36. The method of any one of paragraphs 31-35, wherein step (i) is repeated one or more times in a day.
Paragraph 37. The method of any one of paragraphs 31-36, wherein the composition is administered orally, parenterally, or topically to the subject.
Paragraph 38. The method of any one of paragraphs 31-37, further comprising repeating step (i) every one to two days, or every week, for a time period ranging from 1 week to 8 weeks or from 1 week to 4 weeks.
Paragraph 39. The method of any one of paragraphs 31-33 and 36-38, wherein the health benefit is indicated by:
   (a) visual inspection of cartilage and/or endothelial glycocalyx of the subject using an imaging tool compared to the appearance of the cartilage and/or endothelial glycocalyx prior to step (i); and/or
   (b) visual inspection of cartilage and/or endothelial glycocalyx of the subject using an imaging tool compared to the appearance of the cartilage and/or endothelial glycocalyx following oral administration of a control composition containing the same molar amounts of glucosamine when provided in the control composition in the form of glucosamine sulfate for the same time period.
Paragraph 40. The method of any one of paragraphs 31-33 and 36-39, wherein the health benefit is indicated by:
   (a) a change, wherein the change is an increase or decrease of at least 10%, in the level of one or more biological marker(s) in a biological sample (such as in the blood) of a subject following oral administration of the composition to the subject; and/or
   (b) a change, wherein the change is increase or decrease by at least 10%, in the level of one or more biological marker(s) in a biological sample (such as in the blood) of a subject compared to the oral administration of a control composition containing the same molar amount of glucosamine when provided in the control composition in the form of glucosamine sulfate using the same time period.
Paragraph 41. The method of any one of paragraphs 31-33 and 36-40, wherein the health benefit is indicated by:
   (a) a reduced pain level in the subject as measured using a pain assessment scale compared to the pain level in the subject prior to administering the composition as measured using the same pain assessment scale; and/or
   (b) a lower pain level in the subject as measured using a pain assessment scale compared to the pain level in the subject following oral administration of a control composition containing the same molar amounts of glucosamine when provided in the control composition in the form of glucosamine sulfate using the same time period and measured using the same pain assessment scale.
Paragraph 42. The method of any one of paragraphs 34 and 36-38, wherein the support of the health of the liver is indicated by a change (e.g., an increase or decrease of at least 10%) in the level of one or more biological marker(s) in a biological sample (such as in the blood) of a subject following oral administration of the composition to the subject.
Paragraph 43. The method of any one of paragraphs 35-38, wherein the protection of the liver or reduction of the inflammation in the liver is indicated by a change (e.g., an increase or decrease of at least 10%) in the level of one or more biological marker(s) in a biological sample (such as in the blood) of a subject following oral administration of the composition to the subject.
Paragraph 44. The method of paragraph 42 or 43, wherein the one or more biological marker(s) comprise aspartate aminotransferase (AST), alanine aminotransferase (ALT), gamma-glutamyl transferase (GGT), or one or more inflammatory cytokine(s), or a combination thereof.

The disclosed glucosamine orotate salt, compositions thereof, methods of making thereof, and methods of using thereof can be further understood through the following enumerated paragraphs.
Paragraph 1. A glucosamine orotate salt.
Paragraph 2. A glucosamine orotate salt produced by a process comprising:
   (i) passing glucosamine hydrochloride through a column and into a suspension of orotic acid to form the glucosamine orotate salt,
   wherein the column comprises an ion exchange resin capable of removing chloride ion from glucosamine hydrochloride to produce glucosamine free base, and
   optionally wherein the suspension of orotate acid is under stirring during step (i).
Paragraph 3. The glucosamine orotate salt of paragraph 1 or 2, having a purity of at least 80%, at least 85%, at least 90%, at least 95%, at least 98%, or at least 99%; or in a range from 80% to 100%, from 85% to 100%, from 90% to 100%, from 95% to 100%, from 98% to 100%, or from 99% to 100%.
Paragraph 4. The glucosamine orotate salt of any one of paragraphs 1-3, having a purity of at least 99.8%.
Paragraph 5. The glucosamine orotate salt of any one of paragraphs 1-4, wherein the molar ratio of glucosamine cation and orotate anion in the glucosamine orotate salt is about 1:1.
Paragraph 6. The glucosamine orotate of any one of paragraphs 2-5, wherein the molar ratio of glucosamine hydrochloride to orotic acid in step (i) is about 1:1.
Paragraph 7. The glucosamine orotate salt of any one of paragraphs 2-6, wherein step (i) comprises:
   (a) loading an aqueous solution of glucosamine hydrochloride onto the column, and
   (b) washing the column with deionized water,
   and into the suspension of orotic acid to form the glucosamine orotate salt.
Paragraph 8. The glucosamine orotate salt of any one of paragraphs 2-5, wherein the process further comprises, prior to step (i), (c) washing the ion exchange resin, and (d) loading the washed ion exchange resin into the column.
Paragraph 9. The glucosamine orotate salt of any one of paragraphs 2-8, wherein the process further comprises, prior to step (i), (e) mixing orotic acid in an organic solvent to form the suspension of orotic acid, optionally the mixing in step (e) is performed by stirring.
Paragraph 10. The glucosamine orotate salt of paragraph 9, wherein the organic solvent comprises methanol, ethanol, or both methanol and ethanol.
Paragraph 11. The glucosamine orotate salt of any one of paragraphs 2-10, wherein the process further comprises, after step (i), (f) isolating the glucosamine orotate salt, optionally step (f) is performed by evaporation of the deionized water and organic solvent under vacuum and optionally with warming at a temperature from about 25 °C to about 50 °C.
Paragraph 12. The glucosamine orotate salt of any one of paragraphs 1-11, in a crystal form.
Paragraph 13. The glucosamine orotate salt of paragraph 12, wherein the crystal form is a tetrahydrate crystal form, a dihydrate crystal form, and/or a 0.8 hydrate crystal form.
Paragraph 14. The glucosamine orotate salt of paragraph 12 or 13, wherein the glucosamine orotate salt is stable for at least 1 month, at least 3 months, at least 6 months, at least 1 year, at least 2 years, or at least 3 years, at standard pressure, at a temperature ranging from about 3 °C to about 45 °C, and at a humidity of ≤10% or ranging from about 30% to about 60%.
Paragraph 15. A method for producing glucosamine orotate salt, comprising:
   (i) passing glucosamine hydrochloride through a column and into a suspension of orotic acid to form the glucosamine orotate salt,
   wherein the column comprises an ion exchange resin capable of removing chloride ion from glucosamine hydrochloride to produce glucosamine free base, and
   optionally wherein the suspension of orotate acid is under stirring during step (i).
Paragraph 16. The method of paragraph 15, wherein the molar ratio of glucosamine hydrochloride to orotic acid in step (i) is about 1:1.
Paragraph 17. The method of paragraph 15 or 16, wherein step (i) comprises:
   (a) loading an aqueous solution of glucosamine hydrochloride onto the column, and
   (b) washing the column with deionized water,
   and into the suspension of orotic acid to form the glucosamine orotate salt.
Paragraph 18. The method of any one of paragraphs 15-17, wherein the process further comprises, prior to step (i), (c) washing the ion exchange resin, and (d) loading the washed ion exchange resin into the column.
Paragraph 19. The method of any one of paragraphs 15-18, wherein the process further comprises, prior to step (i), (e) mixing orotic acid in an organic solvent to form the suspension of orotic acid, optionally the mixing in step (e) is performed by stirring.
Paragraph 20. The method of paragraph 19, wherein the organic solvent comprises methanol, ethanol, or both methanol and ethanol.
Paragraph 21. The method of any one of paragraphs 15-20, wherein the process further comprises, after step (i), (f) isolating the glucosamine orotate salt, optionally step (f) is performed by evaporation of the deionized water and organic solvent under vacuum and optionally with warming at a temperature from about 25 °C to about 50 °C.
Paragraph 22. The method of any one of paragraphs 15-21, wherein the molar ratio of glucosamine cation and orotate anion in the glucosamine orotate salt is about 1:1.
Paragraph 23. The method of paragraph 21 or 22, wherein the glucosamine orotate salt is in a crystal form.
Paragraph 24. The method of paragraph 23, wherein the crystal form is a tetrahydrate crystal form, a dihydrate crystal form, and/or a 0.8 hydrate crystal form.
Paragraph 25. The method of paragraph 23 or 24, wherein the glucosamine orotate salt is stable for at least 1 month, at least 3 months, at least 6 months, at least 1 year, at least 2 years, or at least 3 years, at standard pressure, at a temperature ranging from about 3 °C to about 45 °C, and at a humidity of ≤10% or ranging from about 30% to about 60%.
Paragraph 26. A composition comprising:
   the glucosamine orotate salt of any one of paragraphs 1-14; and
   optionally one or more excipients and/or one or more carriers.
Paragraph 27. The composition of paragraph 26, comprising one or more excipients and/or one or more carriers, optionally wherein the one or more excipients comprise non-ionic excipients.
Paragraph 28. The composition of paragraph 26 or 27, wherein the glucosamine orotate salt is present in an effective amount to improve or maintain (a) the health of the endothelial glycocalyx and/or (b) mitochondrial function of a subject.
Paragraph 29. The composition of any one of paragraphs 26-28, wherein the glucosamine orotate salt is present in an effective amount to restore, improve, and/or support the health of blood vessels, and/or to improve maintenance or repair of cartilage in joints or tendons in a subject.
Paragraph 30. The composition of any one of paragraphs 26-28, wherein the glucosamine orotate salt is present in an effective amount to restore, improve, and/or support the health of a liver in a subject.
Paragraph 31. The composition of any one of paragraphs 26-28, wherein the glucosamine orotate salt is present in an effective amount to support glycosaminoglycan and hyaluronan synthesis locally in the skin of a subject.
Paragraph 32. The composition of any one of paragraphs 26-28, wherein the glucosamine orotate salt is present in an effective amount to reduce and/or ameliorate one or more symptom(s) associated with a mitochondrial disease or disorder in a subject.
Paragraph 33. The composition of any one of paragraphs 26-28, wherein the glucosamine orotate salt is present in an effective amount to reduce and/or ameliorate one or more symptom(s) associated with a disease or disorder that is associated with cGAS-STING activation in a subject.
Paragraph 34. The composition of any one of paragraphs 26-33, wherein glucosamine orotate salt is present in an effective amount to promote synthesis of glycosaminoglycans and hyaluronan in a subject.
Paragraph 35. The composition of any one of paragraphs 26-33, wherein glucosamine orotate salt is present in an effective amount to reduce local and/or systemic inflammation in a subject.
Paragraph 36. The composition of any one of paragraphs 26-35, further comprising one or more citrus flavonoids; and/or one or more nitric oxide elevating compounds.
Paragraph 37. The composition of any one of paragraphs 26-36, wherein the composition comprises one or more citrus flavonoids, and wherein the one or more citrus flavonoids comprise diosmin and/or hesperidin.
Paragraph 38. The composition of any one of paragraphs 26-37, wherein the composition comprises one or more nitric oxide elevating compounds, and wherein the one or more nitric oxide elevating compounds comprise arginine, citrulline, nitrate, plant extract(s) with high nitrate content (e.g., beet extract, arugula extract, etc.), isosorbide mononitrate, isosorbide dinitrate, nitroglycerin, nicorandil, molsidomine, and/or phosphodiesterase 5 (PDE5) inhibitor(s) (e.g., sildenafil, vardenafil, tadalafil, etc.).
Paragraph 39. The composition of any one of paragraphs 26-38, in a liquid form or solid form.
Paragraph 40. The composition of any one of paragraphs 26-39, in the form of dry powder, a tablet, a soft or hard chew, a gummy, a pill, or a capsule.
Paragraph 41. The composition of paragraph 39 or 40, wherein the composition is in solid form and further comprises a desiccant.
Paragraph 42. The composition of any one of paragraphs 26-41, in a dosage form for administration to a subject, wherein the glucosamine orotate salt is present in an amount ranging from about 300 mg to about 8 g, from about 300 mg to about 4 g, from about 300 mg to about 1 g, from about 300 mg to about 500 mg, from about 1 g to about 8 g, from about 1 g to about 4 g, from about 2 g to about 8 g, or from about 4 g to about 6 g.
Paragraph 43. A method for providing a health benefit to a subject in need thereof, wherein the health benefit is improving or maintaining the health of the endothelial glycocalyx of the subject, comprising:
   (i) administering the glucosamine orotate salt of any one of paragraphs 1-14 or the composition of any one of paragraphs 26-42 to the subject.
Paragraph 44. A method for providing a health benefit to a subject in need thereof, wherein the health benefit is supporting the health of the blood vessels and/or supporting the synthesis and/or maintenance of cartilage in joints and tendons in the subject, comprising:
   (i) administering the glucosamine orotate salt of any one of paragraphs 1-14 or the composition of any one of paragraphs 26-29 and 34-42 to the subject.
Paragraph 45. A method for providing a health benefit to a subject in need thereof, wherein the health benefit is mitigating pain and/or joint damage due to osteoarthritis in the subject, comprising:
   (i) administering the glucosamine orotate salt of any one of paragraphs 1-14 or the composition of any one of paragraphs 26-29 and 34-42 to the subject.
Paragraph 46. A method for providing a health benefit to a subject in need thereof, wherein the health benefit is supporting the health of the liver in a subject, comprising:
   (i) administering the glucosamine orotate salt of any one of paragraphs 1-14 or the composition of any one of paragraphs 26-28, 30, and 34-42 to the subject.
Paragraph 47. A method for providing a health benefit to a subject in need thereof, wherein the health benefit is protecting the liver against inflammatory damage and/or reducing inflammation in the liver in a subject, comprising:
   (i) administering the glucosamine orotate salt of any one of paragraphs 1-14 or the composition of any one of paragraphs 26-28, 30, and 34-42 to the subject.
Paragraph 48. A method for providing a health benefit to a subject in need thereof, wherein the health benefit is improving or maintaining mitochondrial function of the subject, comprising:
   (i) administering the glucosamine orotate salt of any one of paragraphs 1-14 or the composition of any one of paragraphs 26-42 to the subject.
Paragraph 49. A method for providing a health benefit to a subject in need thereof, wherein the health benefit is reducing and/or ameliorating one or more symptoms associated with a mitochondrial disease or disorder in a subject, comprising:
   (i) administering the glucosamine orotate salt of any one of paragraphs 1-14 or the composition of any one of paragraphs 26-28, 32, and 34-42 to the subject.
Paragraph 50. A method for providing a health benefit to a subject in need thereof, wherein the health benefit is reducing and/or ameliorating one or more symptom(s) associated with a disease or disorder that is associated with cGAS-STING activation in a subject, comprising:
   (i) administering the glucosamine orotate salt of any one of paragraphs 1-14 or the composition of any one of paragraphs 26-28, 33-42 to the subject.
Paragraph 51. The method of any one of paragraphs 43-50, further comprising administering to the subject one or more active agents that modulate monocyte cytokine release.
Paragraph 52. The method of any one of paragraphs 43-51, wherein step (i) is repeated one or more times in a day.
Paragraph 53. The method of any one of paragraphs 43-52, wherein the composition is administered orally, parenterally, or topically to the subject.
Paragraph 54. The method of any one of paragraphs 43-53, further comprising repeating step (i) every one to two days, for a time period ranging from 1 week to 8 weeks or from 1 week to 4 weeks; or repeating step (i) every week, for a time period ranging from 2 to 8 weeks or from 2 to 4 weeks.
Paragraph 55. The method of any one of paragraphs 43, 44, and 51-54, wherein the health benefit is indicated by:
   (a) visual inspection of cartilage and/or endothelial glycocalyx of the subject using an imaging tool compared to the appearance of the cartilage and/or endothelial glycocalyx prior to step (i); and/or
   (b) visual inspection of cartilage and/or endothelial glycocalyx of the subject using an imaging tool compared to the appearance of the cartilage and/or endothelial glycocalyx of a control subject, as measured under the same conditions.
Paragraph 56. The method of any one of paragraphs 43, 44, and 51-55, wherein the health benefit is indicated by:
   (a) a change, wherein the change is an increase or decrease of at least 10%, in the level of one or more biological marker(s) in a biological sample (such as in the blood) of the subject following administration of the glucosamine orotate salt or the composition to the subject; and/or
   (b) a change, wherein the change is increase or decrease by at least 10%, in the level of one or more biological marker(s) in the subject compared to a control subject, as measured under the same conditions.
Paragraph 57. The method of any one of paragraphs 45 and 51-56, wherein the health benefit is indicated by:
   (a) a reduced pain level in the subject as measured using a pain assessment scale compared to the pain level in the subject prior to administering the glucosamine orotate salt or the composition as measured using the same pain assessment scale; and/or
   (b) a lower pain level in the subject as measured using a pain assessment scale compared to the pain level in a control subject using the same pain assessment scale.
Paragraph 58. The method of any one of paragraphs 46 and 51-54, wherein the support of the health of the liver is indicated by, over a 90-day time period:
   maintaining the level of each of one or more biological marker(s) in the subject within a range from a value that is 50% below the lower limit of a normal range to a value that is 50% above the upper limit of a normal range of the respective biological biomarker following administration of the glucosamine orotate salt or the composition to the subject once or twice daily.
Paragraph 59. The method of paragraph 58, wherein the support of the health of the liver is indicated by maintaining the level of each of one or more biological marker(s) in the subject within a normal range of the respective biological biomarker following administration of the glucosamine orotate salt or the composition to the subject.
Paragraph 60. The method of paragraph 58 or 59, wherein the one or more biological marker(s) comprise aspartate aminotransferase (AST), alanine aminotransferase (ALT), total bilirubin, and/or direct bilirubin.
Paragraph 61. The method of any one of paragraphs 47, 48, and 51-54, wherein the protection of the liver or maintained mitochondrial function is indicated by, over a 90-day time period:
   (a) a stable body temperature;
   (b) a level of aspartate aminotransferase (AST) below about 96 U/L and/or a level of alanine aminotransferase (ALT) below about 110 U/L in a biological sample of the subject; and/or
   (c) a normal liver imaging result of the subject using an imaging tool, following administration of the glucosamine orotate salt or the composition to the subject once or twice daily.
Paragraph 62. The method of any one of paragraphs 47 and 51-54, wherein the reduction of the inflammation in the liver is indicated by:
   (a) a lowered level of aspartate aminotransferase (AST) and/or alanine aminotransferase (ALT) in the subject compared to the AST and/or ALT level in the subject prior to administering the composition; and/or
   (b) visual inspection of a liver imaging result of the subject using an imaging tool compared to the result of the subject prior to administering the composition, following administration of the glucosamine orotate salt or the composition to the subject.
Paragraph 63. The method of any one of paragraphs 49-55, wherein the health benefit is indicated by:
   (a) a lowered level of aspartate aminotransferase (AST) and/or alanine aminotransferase (ALT) in the subject compared to the AST and/or ALT level in the subject prior to administering the composition; and/or
   (b) visual inspection of a liver imaging result of the subject using an imaging tool compared to the result of the subject prior to administering the composition,
   following administration of the glucosamine orotate salt or the composition to the subject.

The present invention will be further understood by reference to the following non-limiting examples.

### EXAMPLES

### Example 1: Preparation of Glucosamine Orotate Salt

Organic cation salts of orotic acid, such as arginine orotate, lysine orotate, or meglumine orotate, can be readily produced by mixing a suspension of orotic acid in water with the respective cationic component (e.g., arginine, lysine, or meglumine), with heating and stirring. As the salts are formed, the orotate goes into solution and then isolated by crystallization during cooling. However, this method did not produce glucosamine orotate salt (other than at most a negligible amount).

Amberlite IRA-67 (free base, 812 g) was stirred gently with approximately 2 L deionized water, allowed to settle for approximately 1 min, and the turbid water layer was decanted. The wash was repeated twice, before the water layer appeared clear. The resin was loaded into a 60×5 cm glass column with a 70-100 μm frit. D-glucosamine hydrochloride (21.57 g) was dissolved in 200 mL D/I water and loaded onto the column. The column was washed with D/I water with a total of 1.1 L of D/I water, at which point the eluent had a pH approximately 6-7 as measured by pH strip. The eluent was collected dropwise directly into a flask containing a suspension of orotic acid monohydrate (17.41 g) in 200 mL of methanol while stirring. During the addition of the eluent, the suspension became a clear homogeneous solution. The solution was dried using rotary evaporation and then left in a vacuum desiccator over P₂O₅ to produce 35.4 g of glucosamine orotate salt as an off-white solid. The chemical structures of D-glucosamine hydrochloride, orotic acid monohydrate, and the resulting salt, glucosamine orotate salt, are provided below:

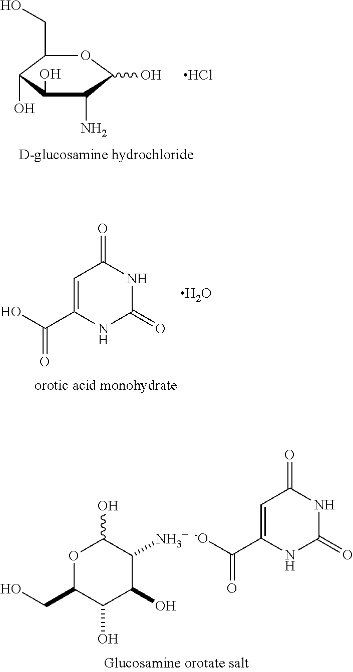

### Example 2: Determination of Water Solubility of Glucosamine Orotate Salt

An aliquot of distilled water was stirred at room temperature (e.g., 25-27 °C at 1 atm). D-glucosamine orotate salt as prepared in Example 1 was slowly added in measured quantities and the mixture was monitored for dissolution. Glucosamine orotate salt in water formed a clear solution at a concentration of 1 gram per ml (1:1 w/v). At higher concentrations, glucosamine orotate salt in water developed a syrup consistency, but without precipitation or visible undissolved particles. The highest concentration achieved in this test was 1.56 g/ml. Glucosamine orotate salt was fully dissolved at this concentration in less than 30 minutes.

The reported water solubility for meglumine orotate salt (N-methylglucosamine orotate) is 5% (1:20 w/v). Solubility of potassium orotate is 1:500 w/v or 0.2% in water. The solubility of free orotic acid in water is approximately 0.1 mg/ml or 1:1000 w/v. Micronization of orotic acid is capable of increasing its solubility by approximately 13-fold, to about 1.3%. The solubility of glucosamine orotate salt surprisingly reached 1.56:1 w/v or 156%, with more glucosamine orotate salt than water in the solution. The water solubility of glucosamine orotate salt is therefore far greater than that of other reported orotate salts.

The pH of a 14.2% solution of glucosamine orotate salt in water (prepared by addition of 0.4 ml of 1.56 g/ml glucosamine orotate salt into 4 ml of water to reduce viscosity) was measured at 3.97 with a calibrated pH meter (Oakton pH550, SN: PH550X23051050), and approximately 4.0 with a “pHydracid Narrow pH range 1-6” pH paper (Micro Essential Laboratory, N.Y., USA).

### Example 3: Glucosamine Orotate Salt for the Treatment of Osteoarthritis

In a subject experiencing symptoms of joint pain due to osteoarthritis, glucosamine orotate salt is administered orally in a daily dose of 5 grams per day, as a tablet for three weeks, and the subject is evaluated for reduced pain perception. The treatment may reduce perceived pain, which is evidence of a slower rate of cartilage degradation. This indicates biosynthesis of glycosaminoglycans and hyaluronan and attenuation of inflammatory tissue damage. Treatment is continued indefinitely because it is not limited by adverse effects. Glucosamine orotate salt is expected to be well-tolerated, not degrade the lining of the gastrointestinal tract, and not carry a substantial sodium chloride load.

The expected benefits of glucosamine orotate salt in osteoarthritis can be determined noninvasively using joint pain rating scales and by X-ray imaging or CT or MRI scans. Suitable imaging methods can be used to detect and measure the thickness and monitor the health of cartilage in a single scan. Additionally, the rate of cartilage thinning can be quantified longitudinally over the course of set time intervals, such as over six to 12 months.

### Example 4: Plasma Pharmacokinetics of Orotate Derived from Orally-Administered Glucosamine Orotate

### Materials and Methods

### Comparison Study

A pharmacokinetic study was performed in mice to measure plasma orotic acid, uridine and uracil in mice after oral administration of D-glucosamine orotate (GO), compared with molar equivalent doses of magnesium orotate (MgOro) and orotic acid (Oro). The latter two forms of orotate have limited water solubility, and very little pharmacokinetic information is available, despite their widespread use as dietary supplements and potential therapeutic agents. Glucosamine orotate displays much greater water solubility than either magnesium orotate or orotic acid, which may improve its absorption after oral administration.

Test drugs were suspended or dissolved in a vehicle containing 0.75% (w/v) hydroxypropyl methylcellulose (HPMC) in water. Mice (CD-1 mice weighing ~25 grams) received oral doses of 500 mg/kg GO, 249.41 mg/kg MgOro, or 232.80 mg/kg Oro by gavage in a volume of 0.01 ml/gram body weight. The molar amount of orotate was the same for all treatments (233 mg/kg orotate), with weight differences due to the counter ions, glucosamine and magnesium.

Mice were randomized to four groups of eight mice each for a total of 32 mice (four per cage), in a manner such that all groups had approximately the same average weight. On the day of the study, food was removed from all cages at 6 a.m., providing time for gastric emptying. The layout for the experiment involved oral gavage treatment of the groups with the compounds or vehicle and obtaining blood samples at 30, 60, 120, and 240 min, with two samples per mouse (Table 1).

TABLE 1
Experiment Layout for Comparison Study

| | No. of | Bleeding Time |
|---|---|---|
| Group ID | Animals | After Dosing |
| GO | 4 | 60 & 240 min |
| | 4 | 30 & 120 min |
| MgOro | 4 | 60 & 240 min |
| | 4 | 30 & 120 min |
| Oro | 4 | 60 & 240 min |
| | 4 | 30 & 120 min |
| HPMC Vehicle | 4 | 60 & 240 min |
| | 4 | 30 & 120 min |

Blood was collected by retroorbital bleeding, under light isoflurane anesthesia, into EDTA plasma collection tubes, and the blood was spun down at 1064 g (10,000 rpm in a 5417R Eppendorf centrifuge with a F-45-30-11 rotor) for 7 minutes to obtain plasma samples, which were frozen on dry ice. Individual blood samples were less than 200 microliters.

Orotate, uridine and uracil were measured in plasma samples after deproteination via HPLC with UV detection. Mice readily convert circulating uridine to uracil, so the sum of uridine plus uracil provides a better index of uridine release from the liver than does measurement of either uridine or uracil alone.

### Dosing Study

Another pharmacokinetic study was performed in mice to measure plasma orotic acid, uridine and uracil in mice after oral administration of glucosamine orotate (GO) at several doses and in several different regimens.

Mice (CD-1 mice weighing ~25 grams) received oral doses of 100, 200 or 400 mg/kg by gavage in a volume of 0.01 ml/gram body weight. GO was dissolved in a vehicle containing 0.75% (w/v) hydroxypropyl methylcellulose (HPMC) in water for oral administration.

Mice were randomized to 4 groups of 12 mice each in a manner such that all groups had approximately the same average body weight. On the day of the study, food was removed from all cages at 6 a.m., providing time for gastric emptying. The layout for the experiment involved oral gavage treatment of the groups with the compounds or vehicle and obtaining blood samples at 30, 60, 120, 180, 240 and 360 minutes, with two samples per mouse (Table 2).

TABLE 2
Experiment Layout for Dosing Study

| | No. of | Bleeding Time |
|---|---|---|
| Group ID | Animals | After Dosing |
| GO400 | 4 | 120 & 360 |
| | 4 | 60 & 240 |
| | 4 | 30 & 180 |
| GO200 | 4 | 120 & 360 |
| | 4 | 60 & 240 |
| | 4 | 30 & 180 |
| GO100 | 4 | 120 & 360 |
| | 4 | 60 & 240 |
| | 4 | 30 & 180 |
| HPMC Vehicle | 4 | 120 & 360 |
| | 4 | 60 & 240 |
| | 4 | 30 & 180 |
| Zero | 4 | Zero Time |

Blood was collected by retroorbital bleeding, under light isoflurane anesthesia, into EDTA plasma collection tubes, and the blood was spun down at 1064 g (10,000 rpm in a 5417R Eppendorf centrifuge with a F-45-30-11 rotor) for 7 minutes to obtain plasma samples, which were frozen on dry ice. Individual blood samples were less than 200 microliters.

Orotate, uridine and uracil were measured in plasma samples after deproteination via HPLC with UV detection. Mice readily convert circulating uridine to uracil, so the sum of uridine plus uracil provides a better index of uridine release from the liver than does measurement of either uridine or uracil alone.

### Results and Discussion

### Comparison Study

Glucosamine Orotate displayed a higher Cₘₐₓ (maximum concentration achieved) and AUC (“area under the curve”, the total amount measured in plasma integrated over time) than did Mg Orotate or Orotic Acid (Table 3). Glucosamine Orotate also yielded a higher concentration of circulating orotate at the 4-hour time point than did the two other compounds (Table 3).

TABLE 3
Circulating Orotate after oral Orotate Compounds Administration

| | | | Concentration |
|---|---|---|---|
| Compound | Cmax | AUC_{(0-14 hours)} | at 4 hours |
| Glucosamine Orotate | 249.6 μM | 35,118 μM × min | 57.9 μM ± 25 |
| Magnesium Orotate | 52.1 μM | 9,948 μM × min | 15.2 μM ± 3.3 |
| Orotic Acid | 144.6 μM | 23,890 μM × min | 21.2 μM ± 6.4 |

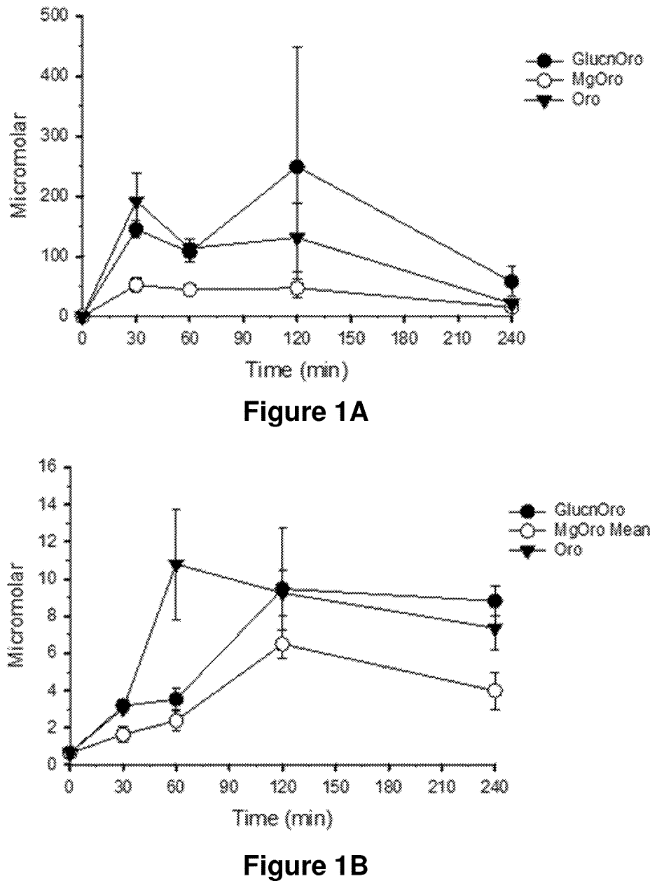

A single dose of each orotate compound resulted in sustained elevation of plasma uridine and uracil, equivalent to about 10 micromolar uridine at steady state (FIG. 1A). Concentrations of uridine after Mg Orotate were lower, in accord with the lower absorption of orotate from this compound (FIG. 1B). Chronic elevation of uridine to about 10 micromolar is sufficient for activation of a range of physiological effects in the periphery. Direct uptake and anabolism of orotate by tissues other than the liver can also augment net elevation of intracellular uridine nucleotides. The dose of glucosamine orotate tested in this study demonstrated its efficacy in disease models.

The mice received doses of orotate of 233 mg/kg (excluding the contribution of counter ions). By standard allometric scaling, this dose should correspond to about 20 mg/kg in humans, which corresponds to a quantity of 1-2 grams of orotate. Therefore, the selected dose in this pharmacokinetic study is in a meaningful range for translation to human physiology.

### Dosing Study

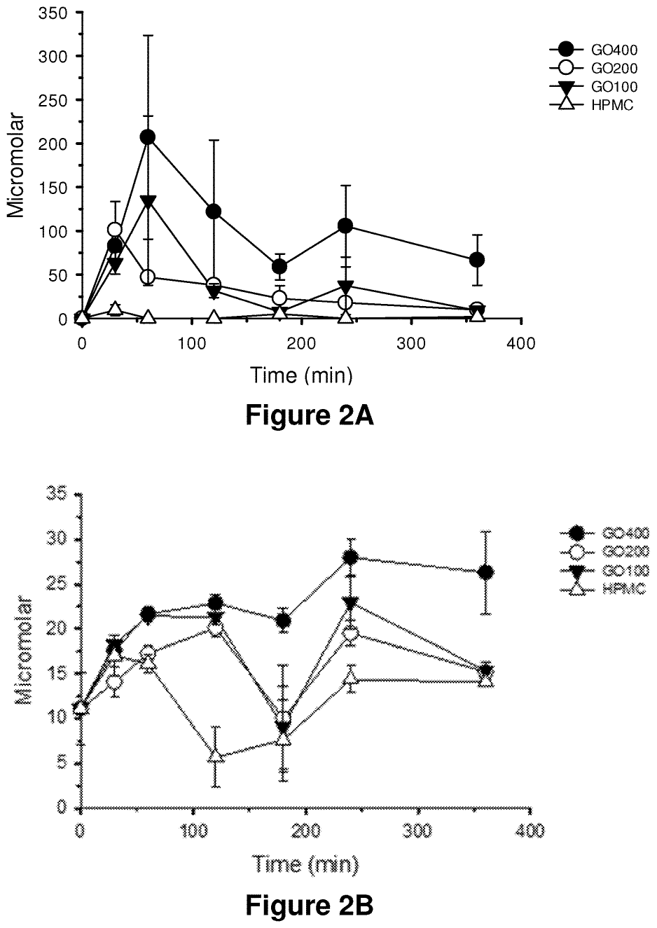

A single dose of each orotate compound resulted in sustained elevation of plasma orotate, uridine and uracil. Glucosamine orotate at 100 and 200 mg/kg behaved similarly, whereas 400 mg/kg resulted in substantially higher concentrations of orotate at all time points (FIG. 2A and Table 4) and also uridine+uracil (FIG. 2B and Table 5), especially at later time points.

TABLE 4
Orotic acid in plasma after oral Glucosamine Orotate administration

| Compound | Cmax | Concentration at 4 hrs | Concentration at 6 hrs |
|---|---|---|---|
| HPMC Vehicle | 9.5 μM | 0 μM ± 0 | 1.9 μM ± 1.9 |
| GO 100 mg/kg | 134.7 μM | 37.4 μM ± 33 | 9.1 μM ± 0.2 |
| GO 200 mg/kg | 101.1 μM | 17.8 μM ± 1.0 | 9.9 μM ± 0.5 |
| GO 400 mg/kg | 206.9 μM | 105 μM ± 47 | 66.7 μM ± 29.9 |

TABLE 5
Uridine + Uracil in plasma after oral Glucosamine Orotate administration

| Compound | Cmax | Concentration at 4 hrs | Concentration at 6 hrs |
|---|---|---|---|
| HPMC Vehicle | 17.0 μM | 14.4 μM ± 1.5 | 14.0 μM ± 0.2 |
| GO 100 mg/kg | 23.0 μM | 23.0 μM ± 3.0 | 15.4 μM ± 0.9 |
| GO 200 mg/kg | 20.1 μM | 19.6 μM ± 1.4 | 15.2 μM ± 0.5 |
| GO 400 mg/kg | 28.0 μM | 28.0 μM ± 2.0 | 26.3 μM ± 4.6 |

The liver, which is the primary organ for de novo synthesis of uridine nucleotides via the de novo pathway, for which orotate is an intermediate, has a high capacity for uptake and anabolism of orotate, mediated both by higher densities of organic acid transporters and anabolic enzymes than in other tissues, along with the kidney to a lesser extent. Phosphoribosyl pyrophosphate (PRPP), metabolically-activated ribose, is also a limiting cofactor for conversion of orotate to uridine nucleotides. Orotate has no metabolic fate other than conversion to uridine nucleotides or excretion, and its anabolism to UMP is not subject to feedback inhibition by uridine nucleotides, unlike earlier steps in the pathway at the level of the CAD enzyme complex or via incorporation of exogenous uridine via uridine kinase. Orotate passing from the gut into the hepatic portal vein is efficiently taken up and its anabolism likely not saturated at the glucosamine orotate doses of 100 and 200 mg/kg, while the higher dose of 400 mg/kg orotate appears to exceed capacity of the liver for rapid uptake, resulting in sustained higher concentrations of circulating orotate. Uridine nucleotides in the liver elevated by exogenous orotate act as a reservoir for release of uridine into the circulation over time, as hepatic uridine nucleotides reduce to normal concentrations. The rate of dephosphorylation of nucleotides and release into the circulation limits the concentration of circulating uridine (and uridine+uracil).

### Example 5: Glucosamine Orotate Protects Against Liver Damage Caused by Bacterial Endotoxin

### Materials and Methods

Female Balb/C mice (eight weeks old with bodyweight >20 grams) were divided into four groups of six animals each. One group of animals was untreated to provide basal values for serum chemistry indices of tissue damage. Mice in the remaining three groups received a single dose of 3 mg/kg Salmonella Typhimurium endotoxin (LPS) by intraperitoneal (IP) injection in a volume 0.01 ml saline per gram of body weight. Prior to endotoxin administration, mice received oral gavage doses of either vehicle (0.75% (w/v) HPMC in water) or glucosamine orotate in a volume of 0.01 ml vehicle per gram body weight. D-Glucosamine orotate (GO) was administered at dose of 522 mg/kg, corresponding to 200 mg/kg of free orotic acid. Eighteen hours after endotoxin administration, blood samples were collected for measurement of serum chemistry indicators of tissue damage. The schedule of treatments is shown in Table 6 below.

TABLE 6
Schedule of Treatments

| Group | 8AM | 11 AM | 1 PM | 7AM Next Day |
|---|---|---|---|---|
| Control (no LPS) | | HPMC | Saline | Blood |
| | | Vehicle | | Collection |
| LPS | | HPMC | LPS 3 | Blood |
| | | Vehicle | mg/kg | Collection |
| LPS + GO 522 mg/kg | | GO 522 | LPS 3 | Blood |
| | | mg/kg | mg/kg | Collection |
| LPS + GO 522 mg/kg | GO 522 | GO 522 | LPS 3 | Blood |
| x2 | mg/kg | mg/kg | mg/kg | Collection |

Blood was collected by retroorbital bleeding under isoflurane anesthesia into serum separator tubes, and the tubes were spun down at 1064 g (10,000 rpm in a 5417R Eppendorf centrifuge with a F-45-30-11 rotor) for 7 minutes to obtain serum samples, which were frozen on dry ice prior to extraction. Serum markers for liver and kidney injury were measured at a commercial preclinical analytical laboratory.

### Results and Discussion

This study was designed to determine whether oral treatment with glucosamine orotate can attenuate inflammatory liver damage caused by bacterial endotoxin (LPS or lipopolysaccharide). Enterally-derived endotoxin leaking into the portal vein from the intestine is a cofactor for initiation and progression of inflammatory liver injury in multiple conditions, including fatty liver disease (MASH or metabolic-associated steatohepatitis), inflammatory bowel disease, alcoholic liver injury and viral hepatitis.

LPS treatment resulted in elevation of the liver transaminases AST and ALT and both Total and Direct (conjugated) Bilirubin, which are all markers for inflammatory liver cell injury. ALT and AST are released from hepatocytes with damage membranes, and serum Bilirubin reflects liver function, as this marker, a hemoglobin degradation product, is removed from the circulation by processing (conjugation) in the liver to make it more water-soluble followed by elimination via bile ducts and urine. LPS also caused hypoglycemia and elevation of Blood Urea Nitrogen (BUN), a marker for kidney injury.

The results of glucosamine orotate treatment are summarized in Table 7 below. Glucosamine orotate treatment resulted in attenuation of the elevation of AST and ALT, and completely prevented elevation of Bilirubin. The mice receiving two doses of glucosamine orotate had higher blood glucose than those treated with LPS plus vehicle or LPS plus a single dose of glucosamine orotate. BUN was not further elevated beyond the LPS effect by either a single or two doses of GO, indicating that plasma orotic acid after administration of these dosages was below a threshold for precipitation in the kidneys.

TABLE 7
Results of Glucosamine Orotate Treatment

| | | | Bilirubin | Bilirubin | | |
|---|---|---|---|---|---|---|
| | AST | ALT | (Total) | (Direct) | Glucose | BUN |
| Treatment | U/L | U/L | mg/dL | mg/dL | mg/dL | mg/dL |
| Normal | 107 ± 18 | 51 ± 16 | 0.5 ± 0.1 | 0.4 ± 0.0 | 189 ± 12 | 15 ± 1 |
| LPS + Vehicle | 695 ± 206 | 869 ± 223 | 1.0 ± 0.4 | 0.9 ± 0.3 | 59 ± 4 | 50 ± 0.8 |
| LPS + GO | 445 ± 151 | 570 ± 154 | 0.5 ± 0.1 | 0.3 ± 0.1 | 57 ± 5 | 56 ± 0.7 |
| 522 mg/kg | | | | | | |
| LPS + GO | 406 ± 182 | 571 ± 237 | 0.5 ± 0.2 | 0.4 ± 0.1 | 77 ± 8 | 51 ± 0.9 |
| 522 mg/kg x2 | | | | | | |

Values are Mean ± SEM
AST = Aspartate Aminotransferase
ALT = Alanine Aminotransferase
BUN = Blood Urea Nitrogen

### Example 6: Dose-Response for Protection with Orally-Administered Glucosamine Orotate Against Liver Damage Caused by Bacterial Endotoxin

The purpose of this study was to determine whether oral treatment with Glucosamine orotate can attenuate inflammatory liver damage caused by bacterial endotoxin (LPS or lipopolysaccharide), comparing doses of 261 mg/kg D-Glucosamine orotate (GO) with 522 mg/kg GO. In human clinical conditions, in addition to LPS exposure due to acute infections with gram-negative bacteria, enterally-derived endotoxin leaking into the portal vein from the intestine is a cofactor for initiation and progression of inflammatory liver injury in multiple conditions, including fatty liver disease (MASH or metabolic-associated steatohepatitis), inflammatory bowel disease, alcoholic liver injury and viral hepatitis.

### Materials and Methods

Female Balb/C mice (eight weeks old with bodyweight >20 grams) were divided into four groups of six animals each. One group of animals was untreated to provide basal values for serum chemistry indices of tissue damage. Mice in the remaining three groups received a single dose of 5 mg/kg Salmonella Typhimurium endotoxin (LPS) by intraperitoneal (IP) injection in a volume 0.01 ml saline per gram of body weight. Prior to endotoxin administration, mice received oral gavage doses of either Vehicle (0.75% HPMC in water; hydroxypropylmethylcellulose) or Glucosamine orotate in a volume of 0.01 ml vehicle per gram body weight. Glucosamine orotate was administered at doses of 261 mg/kg (corresponding to 100 mg/kg free orotic acid), or 522 mg/kg (corresponding to 200 mg/kg of free orotic acid). Groups designated with a “+” in the table received an additional dose of Glucosamine orotate after the LPS injection, at 2 μm. Eighteen hours after endotoxin administration, blood samples were collected for measurement of serum chemistry indicators of tissue damage. The schedule of treatments is shown in Table 8 below.

TABLE 8
Schedule of Glucosamine Orotate (“GO”) Treatments

| | | | | | 7 a.m. Next |
|---|---|---|---|---|---|
| Group | 8 a.m. | 11 a.m. | 1 p.m. | 2 p.m. | Day |
| Control (no | HPMC | HPMC | Saline | Vehicle | Blood |
| LPS) | Vehicle | Vehicle | | | Collection |
| LPS | HPMC | HPMC | LPS 5 | Vehicle | Blood |
| | Vehicle | Vehicle | mg/kg | | Collection |
| LPS + GO | GO 261 | GO 261 | LPS 5 | Vehicle | Blood |
| 261 mg/kg | mg/kg | mg/kg | mg/kg | | Collection |
| LPS + GO | GO 261 | GO 261 | LPS 5 | GO 261 | Blood |
| 261 mg/kg | mg/kg | mg/kg | mg/kg | mg/kg | Collection |
| + | | | | | |
| LPS + GO | GO 522 | GO 522 | LPS 5 | Vehicle | Blood |
| 522 mg/kg | mg/kg | mg/kg | mg/kg | | Collection |
| LPS + GO | GO 522 | GO 261 | LPS 5 | GO 261 | Blood |
| 522 mg/kg | mg/kg | mg/kg | mg/kg | mg/kg | Collection |
| + | | | | | |

Blood was collected by retroorbital bleeding under isoflurane anesthesia into serum separator tubes, and the tubes were spun down at 1064 g (10,000 rpm in a 5417R Eppendorf centrifuge with a F-45-30-11 rotor) for 7 minutes to obtain serum samples, which were frozen on dry ice prior to extraction. Serum markers for liver and kidney injury were measured at a commercial preclinical analytical laboratory.

### Results

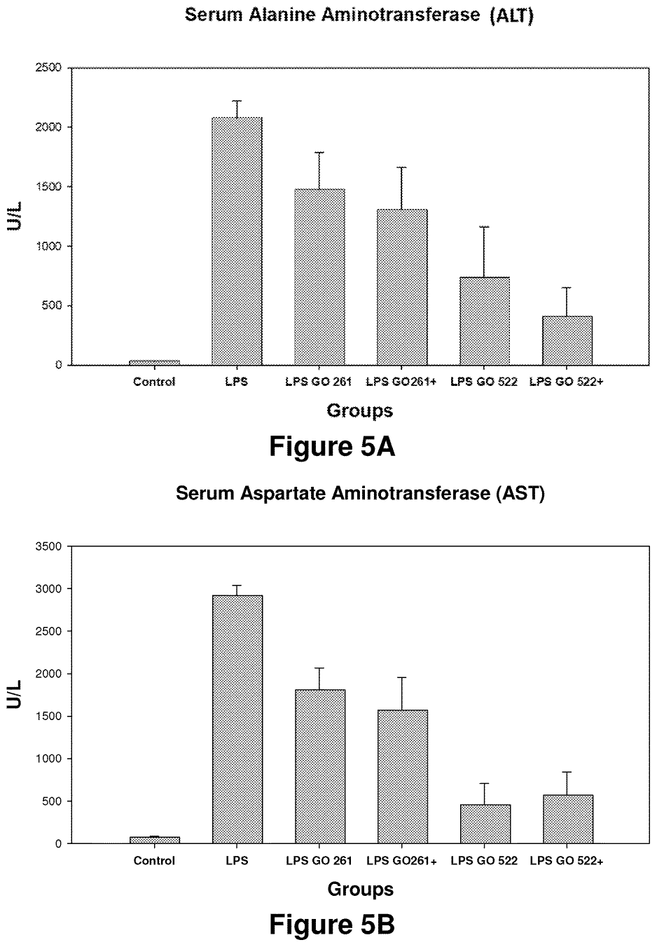

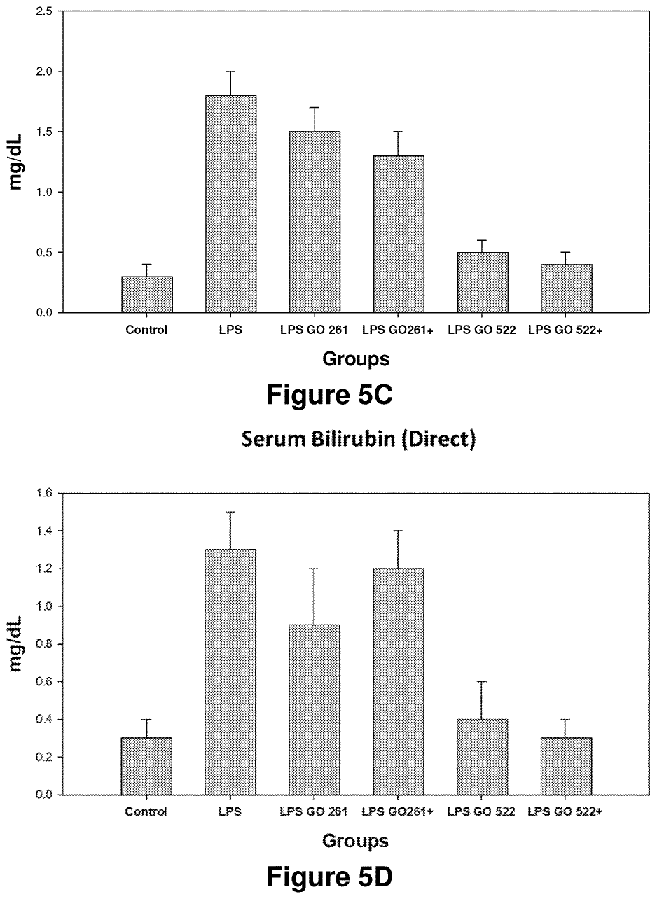

LPS treatment resulted in substantial elevation of the liver transaminases AST and ALT and both Total and Direct (conjugated) Bilirubin, which are all markers for inflammatory liver cell injury. ALT and AST are released from hepatocytes with damage membranes, and serum Bilirubin reflects liver function, as this marker, a hemoglobin degradation product, is removed from the circulation by processing (conjugation with glucuronic acid, yielding “Direct” bilirubin) in the liver to make it more water-soluble followed by elimination via bile ducts and urine. GO treatment resulted in attenuation of the elevation of AST and ALT, and nearly completely prevented elevation of both Total and Direct Bilirubin as shown in FIGS. 5A-5D and Table 9 below.

TABLE 9
Results of Glucosamine Orotate Treatment

| | | | Bilirubin | Bilirubin | |
|---|---|---|---|---|---|
| | ALT | AST | (Total) | (Direct) | Glucose |
| Treatment | U/L | U/L | mg/dL | mg/dL | mg/dL |
| Normal | 34 ± 2 | 77 ± 12 | 0.3 ± 0.1 | 0.3 ± 0.1 | 201 ± 14 |
| LPS + Vehicle | 2078 ± 142 | 2919 ± 121 | 1.8 ± 0.2 | 1.3 ± 0.2 | 76 ± 3 |
| LPS + GO 261 mg/kg | 1477 ± 312 | 1808 ± 258 | 1.5 ± 0.2 | 0.9 ± 0.3 | 73 ± 3 |
| LPS + GO 261 mg/kg + | 1307 ± 356 | 1571 ± 387 | 1.3 ± 0.2 | 1.2 ± 0.2 | 70 ± 3 |
| LPS + GO 522 mg/kg | 735 ± 426 | 459 ± 249 | 0.5 ± 0.1 | 0.4 ± 0.2 | 74 ± 10 |
| LPS + GO 522 mg/kg + | 408 ± 242 | 572 ± 271 | 0.4 ± 0.1 | 0.3 ± 0.1 | 63 ± 6 |

Values are Mean ± SEM
ALT = Alanine Aminotransferase
AST = Aspartate Aminotransferase

In the pharmacokinetic study described in Example 4, plasma orotic acid, uridine and uracil were similar for oral doses of both 261 and 522 mg/kg glucosamine orotate (corresponding to 100 and 200 mg/kg orotic acid respectively), likely because orotate at these doses was predominantly taken up from the portal vein into the liver on first pass and converted to intracellular uridine nucleotides, which was not saturated at 261 mg/kg. Furthermore, there is a threshold effect wherein conversion of exogenous orotate into uridine nucleotides causes feedback inhibition of early steps in de novo uridine nucleotide synthesis at the level of the CAD enzyme complex or salvage of circulating uridine by uridine kinase. Therefore, an administered dose of orotate must be sufficient to overcompensate for inhibited de novo and salvage uridine nucleotide synthesis before total uridine nucleotides are elevated. Augmentation of hepatic uridine nucleotides with orotate via UMP Synthase is not subject to product feedback inhibition, enabling therapeutic elevation of hepatic uridine nucleotides once a threshold dose is exceeded.

The stronger protective activity of 522 mg/kg glucosamine orotate versus 261 mg/kg likely reflects degrees of elevation of total intracellular uridine nucleotides, including UDP-sugars. The approximate equivalence of protection in groups that received an additional dose of glucosamine orotate one hour after LPS shows that the dosing prior to LPS provided long-lasting protection, with blood sampling a full 18 hours after LPS injection and 19 hours after the last dose of glucosamine orotate. Long-lasting protection in this model is consistent with once or twice daily dosing for chronic conditions. A dose of 522 mg/kg in a mouse translates into a 12× reduction due to standard allometric scaling based on body surface area, or 522/12 mg/kg 44 mg/kg. In a 70 kg human subject, 44 mg/kg is a dose of approximately 3 grams of glucosamine orotate (522/12×70=3.045 g).

### Example 7: Glucosamine Orotate Tetrahydrate Analysis

### Materials and Methods

HPLC measurement was performed to assess the purity and HPLC chromatographic properties of Glucosamine Orotate.

The HPLC system included the following modules: QuatPump, Agilent Model G1311A, SN: DE91609832; Thermostatted Auto-Sampler, ALS, Agilent Model G1329A, SN: DE91604864; Thermostatted Column Compartment, TCC, Agilent Model G1316A, SN: DE63073156; Diode Array UV/VIS detector, DAD, Agilent Model G1315B, SN: DE01608992; Prep/Analytical Degasser Model DG-4400 DEGASSEX (Japan), SN: 046B0520. The column used had the following parameters: Zorbax Bonus-RP, Rapid Resolution High Throughput, 4.6×75 mm, 1.8-micron particles, 600 bar, Agilent PN: 830668-901, SN: USCGV01881, LN: B20307. Circular surface area 16.61903 mm².

Parameters of the HPLC gradient method (RP30% D20m37oC0-5 mL.M) are presented in Table 10 below:

TABLE 10
Parameters of HPLC Gradient

| 0, min | 70% C | 30% D | |
|---|---|---|---|
| 1.5 | 70 | 30 | |
| 9.5 | 0 | 100 | P = 94 bar at time “0” |
| 10.5 | 0 | 100 | Column temperature 37° C. |
| 11.5 | 70 | 30 | Auto-sampler tray temperature 20° C. |
| 20 | 70 | 30 | Flow 0.5 mL/min |

Test item solutions and Baseline/background samples were made in HPLC LCMS grade water and injected at a volume of 10 μL. Absorbance was recorded at 210, 230, 260, 280 and 480 nm, with bandwidth 4 nm. Absorbance at 280 nm was used for concentration evaluation and for related calculations. Lab air temperature was 22 °C and relative humidity was 23%.

Orotic acid (uracil-6-carboxylic acid, MW 156.10, anhydrous, Thermo Scientific Cat. No.: 352881000, LOT: 345300, 100 g, received from VWR 9 Jan. 2025) was used as reference standard. D-glucosamine orotate (GO, Lot: 002-16, MW of anhydrous GO=335.3) was used as the test item.

The experimental protocol is as follows:
1. The exact weights of GO and orotic acid were measured on calibrated Analytical balance (Mettler Toledo, Model AG104, SN: 1120141802, Cal Due 30 Nov. 2025).
2. A sequence of freshly made samples of GO and orotic acid in water was prepared in clear glass 20 mL scintillation vials topped with screw-caps/HDPE septa.
3. Orotic acid did not dissolve in water at RT or warm water, but dissolved in hot water at 10 mM, then diluted to 5, 2 and 1 mM.
4. GO dissolved while vortexing in RT water within 1 min at 10 mM, then diluted in a similar way.
5. Solutions were transferred into clear glass, 1.5 mL, High Recovery, Agilent Auto-sampler vials and were put to run on HPLC.

Images of GO powder were examined under an OMAX microscope equipped with digital camera and ScopeImage 9.0 software. Glass slides were 1 mm thick and were cleaned with 70% alcohol prior use.

### Results and Discussion

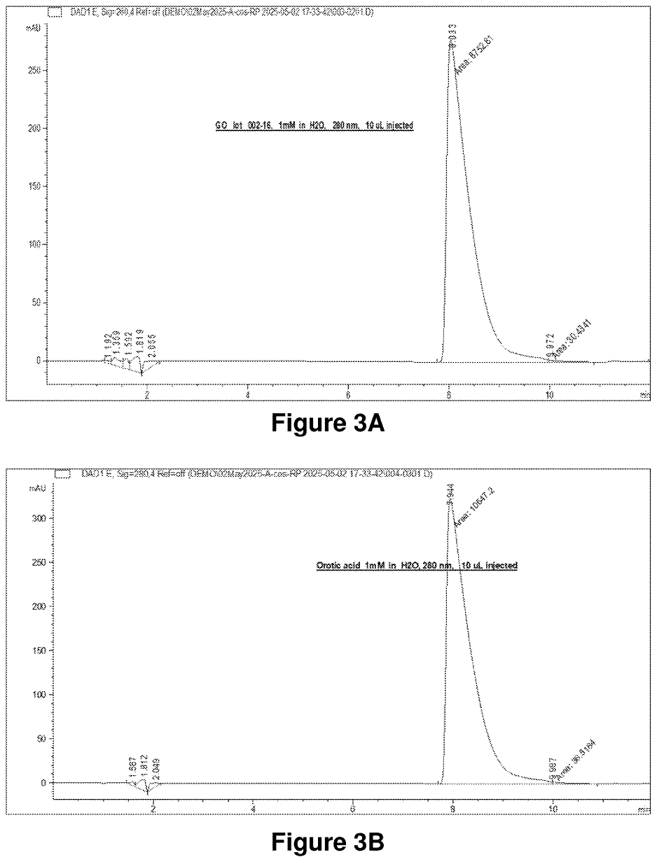

The chromatograms of GO (FIG. 3A) and orotic acid (FIG. 3B) showed peak areas at 280 nm. Relative peak area for orotic acid from GO versus analytical standard orotic acid was calculated as follows:

10647.2/8752.8=1.2164

This provided a factor for calculating the apparent Formula Weight for GO hydrate. The calculated formula weight for anhydrous GO was 335.3 Daltons.

1.2164×335.3 g/mol=407.87 g/mol

The difference between the apparent formula weight for GO versus the orotic acid analytical standard provided a basis for determining whether the weight difference could be due to stoichiometric incorporation of water molecules into the crystal lattice for GO.

407.87 g/mol−335.2 g/mol=72.67 g/mol

The difference between the measured apparent formula weight of GO and the MW for orotic acid was calculated as 72.67 g/mol. No ions or other molecules except water were in contact with GO during its preparation from glucosamine hydrochloride and orotic acid on an ion exchange column. Therefore, this formula weight difference was due to the presence of water molecules. The MW of water is 18 g/mol. Division of 72.67 g/mol by 18 g/mol is 4.037, in agreement with the presence of 4 water molecules per molecule of GO in its crystalline powder form. GO held at room temperature in ambient humidity does not lose or gain weight, indicating stable association of 4 molecules of water per GO unit in its crystalline lattice. Microscopic images of GO powder confirmed the crystalline powder form of GO, as illustrated in FIGS. 4A and 4B.

Glucosamine sulfate and other glucosamine salts are unstable, as is free glucosamine without a counter ion. Glucosamine sulfate is hygroscopic, capturing moisture from the atmosphere that results in glucosamine sulfate turning into a paste consistency and undergoing oxidative degradation which yields brown products. The instability of glucosamine sulfate was addressed by addition of sodium chloride, with assertions that the NaCl formed a stable complex with glucosamine sulfate. However, later studies indicated that combining glucosamine sulfate with NaCl simply results in formation of mixtures of separate crystals of glucosamine hydrochloride and sodium sulfate, not stable complexes of glucosamine sulfate with stoichiometric amounts of NaCl.

Glucosamine orotate produced by the method described in Example 1 is a white powder that is stable at room temperature in open air. One factor underlying its stability may be that the crystalline lattice of GO includes water molecules present in stoichiometric whole numbers of water molecules per molecule of GO. HPLC analysis showed that the discrepancy between GO content of the powder as determined by gravimetric measurement versus assay by HPLC was in close agreement with stoichiometric inclusion of four molecules of water per molecule of GO. Moisture generally destabilizes glucosamine and glucosamine salts, but in the case of GO, water molecules may fill holes in the crystal lattice structure of GO powder, yielding stable glucosamine orotate tetrahydrate. This represents the first report of stoichiometric hydrate salts of glucosamine.

### Example 8: Effects of Glucosamine Orotate Salt on Hypothermia and Liver Injury Induced by Bacterial Endotoxin

### Materials and Methods

This study was designed to assess core body temperature before and after administration of bacterial endotoxin (Salmonella typhimurium LPS, 6 mg/kg i.p.) in mice treated with D-Glucosamine orotate (GO) (522 mg/kg or 200 mg/kg orotate equivalent) or Vehicle prior to LPS administration. Blood samples were collected for measurement of serum markers of tissue damage 18 hours after LPS administration, focusing on the liver. The treatment schedule is shown in Table 11 below.

TABLE 11
Schedule of GO/Vehicle Treatments

| Group | Day 1 | | Day 2 | | | Day 3 |
|---|---|---|---|---|---|---|
| (n = 10/grp) | 8 AM | 1 PM | 8AM | 11 AM | 1 PM | 7AM |
| LPS + | Vehicle | Vehicle | Vehicle | Vehicle | LPS | Blood |
| Vehicle | p.o. | p.o. | p.o. | p.o. | 6 mg/kg | Collection |
| | | | | | i.p. | |
| LPS + GO | GO p.o. | GO p.o. | GO p.o. | GO p.o. | LPS | Blood |
| 522 mg/kg | | | | | 6 mg/kg | Collection |
| | | | | | i.p. | |

The rectal temperature of the animals was measured one hour before LPS injection (normal temperature) and 18 hours after LPS injection, immediately before blood collection by retroorbital bleeding under isoflurane anesthesia into serum separator tubes. After spontaneous clotting, the tubes were centrifuged at 1064 g (10,000 rpm in a 5417R Eppendorf centrifuge with a F-45-30-11 rotor) for 7 minutes to obtain serum samples, which were frozen on dry ice for storage and transport for analysis.

### Results and Discussion

Mice or rats undergo impairments in body temperature regulation during endotoxin-induced systemic inflammation, initially with fever followed by profound hypothermia as a physiological response to survive circulatory shock and impaired oxygen utilization secondary to mitochondrial failure. During LPS shock, the body temperature of mice can equilibrate with room temperature. This hypothermia prolongs survival time versus warming affected mice toward normal body temperature, since the hypothermia is protective in settings of low capacity for oxygen delivery or utilization. However, mice with serious hypothermia after endotoxin do not recover and die within about 24 hours after the LPS.

Mitochondria are important for maintaining body temperature and preventing hypothermia, expending a large fraction of their potential energy to provide body heat, at the expense of generation of ATP for other purposes. LPS causes depletion of mitochondrial DNA in the liver, resulting in both failure of oxidative phosphorylation and also activation of secondary inflammation due to leakage of mitochondrial DNA fragments into the cytosol, where they can activate the cGAS-STING signaling pathway. cGAS-STING activates interferon signaling and other inflammatory pathways as an antiviral response to cytosolic double-stranded DNA, which can be pathologically activated by ectopic mitochondrial DNA, exacerbating inflammation and tissue damage.

In this study, mice treated with LPS plus GO felt warmer to the touch than did mice treated with LPS plus Vehicle. Studies were conducted to measure core body temperatures (Table 12) and correlate them with circulating markers for tissue damage and protection (Table 13).

The data in Table 12 shows that mice treated with intraperitoneal LPS plus oral vehicle displayed severe hypothermia, with average body temperature nearly 20 degrees F lower than normal baseline temperature recorded prior to LPS. The mice in this group were immobile in their cages. In contrast, mice pretreated with oral GO prior to LPS maintained an average body temperature only about 3 degrees F lower than normal, and mice in this group were capable of walking in their cages. GO treatment maintained substantial capacity for energy metabolism and thermogenesis, which may involve both better hemodynamics and especially prevention of mitochondrial failure in the liver and possibly other organs as well, including skeletal muscle.

TABLE 12
Core Body Temperatures of Mice Treated with GO vs. Vehicle

| | Core Body Temperature (Degrees F.) | |
|---|---|---|
| | Group | |
| | Before LPS | 18 hr after LPS |
| | Mean ± SEM | Mean ± SEM |
| LPS + Vehicle | 98.1 ± 0.2 | 79.2 ± 0.8 |
| LPS + GO | 98.9 ± 0.5 | 95.1 ± 2.1 |

The data in Examples 8-14 was each obtained from a different experiment. It is understood that the core body temperature of mice is affected by the surrounding temperatures. Small changes in housing temperature for mice can lead to considerable changes in recorded body temperature. As the housing temperatures varied among different experiments, it was expected that the core body temperature data of mice among different experiments fluctuated as a result of differences in housing temperatures. Regardless, the core body temperature data of mice from the same experiment was comparable and meaningful, with good internal consistency within single experiments.

The data in Table 13 shows that GO treatment protected the livers of mice against inflammatory injury due to LPS administration, reducing elevations in the transaminases ALT and

AST and in both Direct (conjugated) and Total Bilirubin.

TABLE 13
Circulating Markers of Mice Treated with GO vs. Vehicle

| | | | Bilirubin | Bilirubin |
|---|---|---|---|---|
| | | | Total | Direct |
| | ALT U/L | AST U/L | mg/dL | mg/dL |
| | Hepatocyte Damage | | Liver Function | |
| Normal (no LPS) | 34 ± 2 | 77 ± 12 | 0.30 ± 0.10 | 0.30 ± 0.10 |
| LPS + Vehicle | 1311 ± 295 | 1512 ± 341 | 0.74 ± 0.15 | 0.85 ± 0.16 |
| LPS + GO | 86 ± 9 | 148 ± 22 | 0.32 ± 0.03 | 0.37 ± 0.04 |

ALT = Alanine Aminotransferase
AST = Aspartate Aminotransferase

### Example 9: Comparison of Glucosamine Orotate Versus Orotic Acid or Glucosamine HCL Against LPS-Induced Hypothermia and Liver Damage

### Materials and Methods

Female Balb/C Mice (8 weeks old, weighing >20 grams) housed in a room held at 72-73 degrees F were randomly divided into treatment groups (n=8 mice/group), though with similar average body weights per cage.

Mice each received four oral gavage doses of treatment agents, 2 doses on the day before LPS and 2 doses on the morning of LPS treatment at 8 AM and 11 AM. 6 mg/kg LPS (Salmonella typhimurium endotoxin) was administered by intraperitoneal injection at 1 PM. The following morning, 18 hours after LPS injection, rectal body temperatures were measured with a digital thermocouple thermometer. Mice were then anesthetized with isoflurane and blood samples were collected into serum separator tubes. Following coagulation and centrifugation, the resulting serum was analyzed for serum chemistry markers for liver and kidney injury. The treatment schedule is shown in Table 14 below.

TABLE 14
Schedule of GO, Vehicle, Orotic Acid, and Glucosamine HCl Treatments

| | | | Day 2 | | | |
|---|---|---|---|---|---|---|
| | Day 1 | | | Group | | Day 3 |
| Group (n = 8/grp) | 8 AM | 1 PM | 8AM | (n = 8/grp) | 8 AM | 1 PM |
| Control (no LPS) | HPMC | HPMC | HPMC | Control | HPMC | HPMC |
| | Vehicle | Vehicle | Vehicle | (no LPS) | Vehicle | Vehicle |
| 8 Am and | HPMC | HPMC | HPMC | 8 Am and | HPMC | HPMC |
| 11 AM LPS + | Vehicle | Vehicle | Vehicle | 11 AM LPS + | Vehicle | Vehicle |
| Vehicle | | | | Vehicle | | |
| LPS + GO | Go | GO | GO | LPS + GO | Go | GO |
| 522 mg/kg | | | | 522 mg/kg | | |
| LPS + Orotate | Orotate | Orotate | Orotate | LPS + Orotate | Orotate | Orotate |
| 200 mg/kg | | | | 200 mg/kg | | |
| LPS + Orotate | Orotate + | Orotate + | Orotate + | LPS + Orotate | Orotate + | Orotate + |
| 200 mg/kg + | GcN | GIN | GlcN | 200 mg/kg + | GlcN | GicN |
| GlcN | | | | GIN | | |
| 322 mg/kg | | | | 322 mg/kg | | |

GO = D-Glucosamine Orotate
Oro or Orotate = Orotic Acid
GlcN = Glucosamine HCl
LPS = Lipopolysaccharide; endotoxin from Salmonella typhimurium

### Results and Discussion

As demonstrated in Examples 2 and 4 above, GO displays better water solubility than do other salts of orotic acid and consequently delivers orotate into the circulation more effectively. Furthermore, GO co-delivers equimolar amounts of glucosamine with the orotate, and glucosamine displays beneficial biochemical and pharmacological activity in models of LPS-induced liver injury, including improvement of survival.

The data in Table 15 shows that oral GO was more effective than were equimolar amounts of each of its constituents, orotic acid or glucosamine as well as a mixture of the two agents, which did, however, outperform the individual components orotic acid and glucosamine HCl. It is likely that one facet of the greater efficacy of GO is due to better absorption related to the much higher water solubility of GO versus orotic acid (>1:1 vs ~1:10,000 respectively). Orotic acid and glucosamine HCl do not readily form soluble GO when simply mixed in water, so the effect of glucosamine on efficacy may also involve biochemical effects of glucosamine in addition to its solubilizing effect as a component of GO.

TABLE 15
Effects of GO, Orotic Acid, and Glucosamine
HCl on body temperatures of mice 18 hours
after administration of S. typhimurium endotoxin

| | Core Body Temperature (Degrees F) | |
|---|---|---|
| Group | Mean ± SEM | Median |
| Normal (no LPS) | 99.2 ± 0.2 | 98.9 |
| LPS + Vehicle | 76.7 ± 0.4 | 76.7 |
| LPS + GO | 87.8 ± 2.5 | 86.7 |
| LPS + Orotic Acid | 79.5 ± 1.9 | 77.1 |
| LPS + Orotic Acid + | 83.9 ± 3.3 | 79.9 |
| Glucosamine | | |
| LPS + Glucosamine | 76.3 ± 0.3 | 76.6 |

The data in Table 16 shows that LPS treatment resulted in substantial elevation of the liver transaminases AST and ALT and both Total and Direct (conjugated) Bilirubin, which are all markers for inflammatory liver cell injury. ALT and AST are released from hepatocytes with damaged membranes, and serum Bilirubin reflects liver function, as this marker, a hemoglobin degradation product, is removed from the circulation by processing (conjugation with glucuronic acid, yielding “Direct” bilirubin) in the liver to make it more water-soluble followed by elimination via bile ducts and urine. GO treatment resulted in attenuation of the elevation of AST and ALT, and nearly completely prevented elevation of both Total and Direct Bilirubin.

TABLE 16
Serum chemistry markers of Mice 18 hours after
administration of S. typhimurium endotoxin

| | | | TOTAL | BILI- |
|---|---|---|---|---|
| | | | BILI- | RUBIN |
| | ALT | AST | RUBIN | DIRECT |
| Groups | U/L | U/L | mg/dL | mg/dL |
| Control | 43 ± 13 | 77 ± 20 | 0.3 ± 0.0 | 0.2 ± 0.0 |
| (Normal) | | | | |
| LPS + Vehicle | 1558 ± 57 | 1566 ± 76 | 1.9 ± 0.1 | 1.5 ± 0.1 |
| LPS + GO | 207 ± 62 | 317 ± 80 | 0.4 ± 0.1 | 0.4 ± 0.1 |
| LPS + Oro | 648 ± 153 | 701 ± 134 | 0.8 ± 0.1 | 0.6 ± 0.1 |
| LPS + GlcN | 1581 ± 130 | 1686 ± 106 | 1.2 ± 0.1 | 0.9 ± 0.1 |
| LPS + Oro + | 861 ± 282 | 976 ± 283 | 0.7 ± 0.1 | 0.5 ± 0.1 |
| GlcN | | | | |

ALT = Alanine Aminotransferase;
AST = Aspartate Aminotransferase

The data in Table 17 shows that LPS also caused hypoglycemia and elevation of Blood Urea Nitrogen (BUN), a marker for kidney injury. High dosages of orotic acid can accumulate in the kidneys, leading to kidney damage that can be measured using the markers shown in Table 17. BUN was not further elevated beyond the LPS damage after treatment with GO, indicating that plasma orotate after administration of GO was below a threshold for precipitation in the kidneys. Glucose and creatinine did not elevate above the LPS damage after administration of GO. Further, the levels of creatinine, glucose, and BUN in mice treated with GO were comparable to those in mice treated with Orotic acid and GlcN respectively. These results demonstrated that GO did not cause further kidney damage in mice beyond the damage cause by LPS.

TABLE 17
Creatinine, Glucose, and BUN levels 18 hours after
administration of S. typhimurium endotoxin.

| | Creatinine | GLUCOSE | BUN |
|---|---|---|---|
| Mean | mg/dl | mg/dL | mg/dL |
| Control (Normal) | 0.31 ± 0.06 | 212 ± 13 | 20 ± 1 |
| LPS + Vehicle | 0.51 ± 0.03 | 63 ± 10 | 63 ± 8 |
| LPS + GO | 0.50 ± 0.08 | 52 ± 7 | 74 ± 5 |
| LPS + Oro | 0.65 ± 0.07 | 55 ± 5 | 89 ± 5 |
| LPS + GlcN | 0.62 ± 0.02 | 76 ± 7 | 69 ± 3 |
| LPS + Oro + GlcN | 0.75 ± 0.12 | 66 ± 13 | 65 ± 8 |

BUN = Blood Urea Nitrogen

These results show that GO was more effective than orotic acid alone for prevention of AST and ALT elevation in response to systemic inflammation induced by LPS. Glucosamine alone had no apparent effect in this model and did not improve the effect of orotic acid on these measurements. However, Glucosamine alone did reduce the rise in both Total and Direct Bilirubin caused by LPS, showing some protection of liver function. Furthermore, Glucosamine also appeared to improve the beneficial effect of orotic acid on maintenance of body temperature after LPS, though without a significant effect of Glucosamine alone on body temperature.

### Example 10: Glucosamine Orotate versus Orotic Acid, Magnesium Orotate or Potassium Orotate against LPS-Induced Hypothermia and Liver Damage

### Materials and Methods

Female Balb/C Mice (8 weeks old, weighing >20 grams) housed in a room held at 72-73 degrees F were randomly divided into treatment groups (n=8 mice/group), though with similar average body weights per cage.

Equimolar doses of GO, orotic acid, magnesium orotate, or potassium orotate were administered to groups of 8 mice each as indicated in Table 18 below.

Mice each received four oral gavage doses of treatment agents, 2 doses on the day before LPS and 2 doses on the morning of LPS treatment at 8 AM and 11 AM. 6 mg/kg LPS (Salmonella typhimurium endotoxin) was administered by intraperitoneal injection at 1 PM. The following morning, 18 hours after LPS injection, rectal body temperatures were measured with a digital thermocouple thermometer. Mice were then anesthetized with isoflurane and blood samples were collected into serum separator tubes. Following coagulation and centrifugation, the resulting serum was analyzed for serum chemistry markers for liver and kidney injury. The treatment schedule is shown in Table 19 below.

TABLE 18
GO, Vehicle, Orotic Acid, Magnesium
Orotate, and Potassium Orotate Groups

| | Number of | Compound | LPS |
|---|---|---|---|
| Group | Mice | (mg/Kg) or HPMC | (6 mg/Kg) |
| GO (D-Glucosamine | 8 | 521.9 | ✓ |
| Orotate) | | | |
| Orotic Acid | 8 | 200 | ✓ |
| Mg Orotate | 8 | 214.3 | ✓ |
| K Orotate | 8 | 248.8 | ✓ |
| Vehicle + LPS | 8 | HPMC | ✓ |
| Vehicle no LPS | 3 | HPMC | Saline |

GO = D-Glucosamine Orotate
Mg Orotate = Magnesium Orotate
K Orotate = Potassium Orotate
LPS = Lipopolysaccharide; endotoxin from Salmonella typhimurium

TABLE 19
Schedule of GO, Vehicle, Orotic Acid, Magnesium Orotate,
and Potassium Orotate Treatments

| Group | Day 1 | | Day 2 | | | Day 3 |
|---|---|---|---|---|---|---|
| (n = 8/grp) | 8 AM | 1 PM | 8 AM | 11 AM | 1 PM | 7AM |
| Normal | HPMC | HPMC | HPMC | HPMC | Saline | Blood |
| (no LPS) | Vehicle | Vehicle | Vehicle | Vehicle | | Collection |
| LPS + | HPMC | HPMC | HPMC | HPMC | LPS | Blood |
| Vehicle | Vehicle | Vehicle | Vehicle | Vehicle | 6 mg/kg | Collection |
| LPS + GO | Go | GO | GO | GO | LPS | Blood |
| | | | | | 6 mg/kg | Collection |
| LPS + | Orotate | Orotate | Orotate | Orotate | LPS | Blood |
| Orotic Acid | | | | | 6 mg/kg | Collection |
| LPS + Mg | Mg | Mg | Mg | Mg | LPS | Blood |
| Orotate | Orotate | Orotate | Orotate | Orotate | 6 mg/kg | Collection |
| LPS + K | K | K | K | K | LPS | Blood |
| Orotate | Orotate | Orotate | Orotate | Orotate | 6 mg/kg | Collection |

### Results and Discussion

The data in Table 20 shows that intraperitoneal LPS with oral treatments of Vehicle resulted in severe hypothermia, with a core body temperature of 77 degrees F, approximately 20 degrees F below that of normal mice. GO treatment attenuated LPS-induced hypothermia, with a mean temperature of 91 degrees F. Orotic acid also attenuated the drop in body temperature caused by LPS, whereas neither magnesium orotate nor potassium orotate counteracted LPS-induced hypothermia at 18 hours.

TABLE 20
Effects of GO versus other orotate salts on body temperatures of
mice 18 hours after administration of S. typhimurium endotoxin

| | Core Body Temperature (Degrees F) | |
|---|---|---|
| Group | Mean ± SEM | Median |
| Normal (no LPS) | 98.0 ± 0.5 | 97.8 |
| LPS + Vehicle | 77.1 ± 0.4 | 76.9 |
| LPS + GO | 91.0 ± 3.0 | 92.8 |
| LPS + Orotic Acid | 88.7 ± 3.0 | 87.3 |
| LPS + Mg Orotate | 79.2 ± 6.0 | 75.8 |
| LPS + K Orotate | 75.6 ± 0.3 | 75.4 |

The data in Table 21 shows that LPS treatment resulted in substantial elevation of the liver transaminases AST and ALT and both Total and Direct (conjugated) Bilirubin, which are all markers for inflammatory liver cell injury. GO treatment resulted in attenuation of the elevation of AST and ALT, and of both Total and Direct Bilirubin. Orotic acid was less effective than GO for preventing elevation of the serum markers of liver injury, and Mg Orotate and K Orotate were both less effective than was GO or orotic acid.

TABLE 21
Effects of GO versus other orotate salts on serum chemistry markers of
Mice 18 hours after administration of S. typhimurium endotoxin

| | | | TOTAL | BILIRUBIN |
|---|---|---|---|---|
| Groups | ALT | AST | BILIRUBIN | DIRECT |
| N = 6/group | U/L | U/L | mg/dL | mg/dL |
| Control (Normal) | 43 ± 13 | 77 ± 20 | 0.3 ± 0.0 | 0.2 ± 0.0 |
| LPS + Vehicle | 1380 ± 174 | 1449 ± 171 | 1.7 ± 0.1 | 1.4 ± 0.1 |
| LPS + GO | 423 ± 28 | 496 ± 173 | 0.5 ± 0.1 | 0.4 ± 0.1 |
| LPS + Orotate | 1049 ± 297 | 1245 ± 338 | 0.9 ± 0.2 | 0.7 ± 0.2 |
| LPS + Mg Orotate | 1581 ± 130 | 1005 ± 332 | 1.3 ± 0.3 | 1.0 ± 0.3 |
| LPS + K Orotate | 1550 ± 174 | 1655 ± 196 | 1.7 ± 0.2 | 1.3 ± 0.2 |

ALT = Alanine Aminotransferase;
AST = Aspartate Aminotransferase

### Example 11: Comparison of Glucosamine Orotate Versus Calcium Orotate or Sodium Orotate Against LPS-Induced Hypothermia and Liver Damage

### Materials and Methods

Female Balb/C Mice (8 weeks old, weighing >20 grams) housed in a room held at 72-73 degrees F were randomly divided into treatment groups (n=8 mice/group), though with similar average body weights per cage.

Equimolar doses of GO, orotic acid, calcium orotate, or sodium orotate as indicated in Table 22 below were administered to groups of 8 mice each.

Mice each received four oral gavage doses of treatment agents, two on the day before LPS at 8 AM and 1 PM, and 2 doses on the morning of LPS treatment at 8 AM and 11 AM. 6 mg/kg LPS (Salmonella typhimurium endotoxin) was administered by intraperitoneal injection at 1 PM. The following morning, 18 hours after LPS injection, rectal body temperatures were measured with a digital thermocouple thermometer. Mice were then anesthetized with isoflurane and blood samples were collected into serum separator tubes. Following coagulation and centrifugation, the resulting serum was analyzed for serum chemistry markers for liver and kidney injury. The treatment schedule is shown in Table 23 below.

TABLE 22
GO, Vehicle, Orotic Acid, Calcium
Orotate, or Sodium Orotate Groups

| | Number | Oral Compound | LPS |
|---|---|---|---|
| Group | of Mice | (mg/Kg) or HPMC | (6 mg/Kg i.p.) |
| GO | 8 | 521.88 | ✓ |
| CaOro | 8 | 224.38 | ✓ |
| NaOro | 8 | 228.16 | ✓ |
| HPMC-LPS | 8 | HPMC | ✓ |
| Normal | 3 | HPMC | — |
| Control | | | |

GO = D-Glucosamine Orotate
Ca Orotate = Calcium Orotate
Na Orotate = Sodium Orotate
LPS = Lipopolysaccharide; endotoxin from Salmonella typhimurium

TABLE 23
Schedule of GO, Vehicle, Orotic Acid, Calcium Orotate,
or Sodium Orotate Treatments

| Group | Day 1 | | Day 2 | | | Day 3 |
|---|---|---|---|---|---|---|
| (n = 8/grp) | 8 AM | 1 PM | 8 AM | 11 AM | 1 PM | 7 AM |
| Normal | HPMC | HPMC | HPMC | HPMC | Saline | Blood |
| (no LPS) | Vehicle | Vehicle | Vehicle | Vehicle | | Collection |
| LPS + | HPMC | HPMC | HPMC | HPMC | LPS | Blood |
| Vehicle | Vehicle | Vehicle | Vehicle | Vehicle | | Collection |
| LPS + GO | Go | GO | GO | GO | LPS | Blood |
| | | | | | | Collection |
| LPS + Ca | Ca | Ca | Ca | Ca | LPS | Blood |
| Orotate | Orotate | Orotate | Orotate | Orotate | | Collection |
| LPS + K | Na | Na | Na | Na | LPS | Blood |
| Orotate | Orotate | Orotate | Orotate | Orotate | | Collection |

### Results and Discussion

Metal salts of orotic acid deliver not only orotate into the bloodstream, but also the counter ions, which may have adverse effects in some conditions. The relative efficacies of sodium and calcium salts of orotate compared to Glucosamine Orotate were tested in the LPS model of liver injury.

The data in Table 24 shows that intraperitoneal LPS with oral treatments of HPMC Vehicle resulted in severe hypothermia, with a core body temperature of 80.6 F, about 17 degrees F below that of normal mice. Oral GO treatment attenuated LPS-induced hypothermia, with a mean temperature of 82.3 F. Calcium orotate and sodium orotate both exacerbated LPS-induced hypothermia at 18 hours after LPS.

TABLE 24
Effects of GO vs Ca Orotate or Na Orotate on body temperatures of
mice 18 hours after administration of S. typhimurium endotoxin

| | Core Body Temperature |
|---|---|
| | (Degrees F) |
| Group | Mean ± SEM |
| Normal (no LPS) | 98.0 ± 0.5 |
| LPS + Vehicle | 80.6 ± 2.7 |
| LPS + GO | 82.3 ± 2.8 |
| LPS + Ca Orotate | 76.9 ± 0.5 |
| LPS + Na Orotate | 77.9 ± 0.4 |

The data in Table 25 shows that LPS treatment resulted in elevation of the liver transaminases AST and ALT and both Total and Direct (conjugated) Bilirubin. GO treatment resulted in attenuation of the elevation of AST and ALT, and of both Total and Direct Bilirubin. In contrast, Ca Orotate and Na Orotate exacerbated rather than ameliorated LPS-induced elevation of the tested markers for liver injury. These counterproductive effects are likely due to delivery of excessive amounts of the Calcium and Sodium counter ions for orotate, rather than to poor delivery of orotate, for example due to low water solubility of Ca orotate and Na orotate, which would not be expected to result in exacerbation of liver injury by LPS. Therefore, GO is more active than either Ca orotate or Na orotate, and is furthermore safer than the metal ion salts in the LPS model of liver injury.

TABLE 25
Serum chemistry markers 18 hours after administration
of S. typhimurium endotoxin

| | | | TOTAL | BILIRUBIN |
|---|---|---|---|---|
| Groups | ALT | AST | BILIRUBIN | DIRECT |
| N = 6/group | U/L | U/L | mg/dL | mg/dL |
| Control (Normal) | 43 ± 13 | 77 ± 20 | 0.3 ± 0.0 | 0.2 ± 0.0 |
| LPS + Vehicle | 800 ± 170 | 907 ± 162 | 0.7 ± 0.1 | 0.9 ± 0.1 |
| LPS + GO | 466 ± 155 | 580 ± 169 | 0.6 ± 0.2 | 0.8 ± 0.2 |
| LPS + Ca Orotate | 980 ± 221 | 1113 ± 284 | 1.0 ± 0.2 | 1.2 ± 0.2 |
| LPS + Na Orotate | 1281 ± 186 | 1407 ± 201 | 1.1 ± 0.2 | 1.3 ± 0.2 |

ALT = Alanine Aminotransferase;
AST = Aspartate Aminotransferase

### Example 12: Comparison of Glucosamine Orotate Alone or in Combination with Arginine or Citrulline Against LPS-Induced Hypothermia and Liver Damage

### Materials and Methods

Female Balb/C Mice (8 weeks old, weighing >20 grams) housed in a room held at 72-73 degrees F were randomly divided into treatment groups (n=8 mice/group), though with similar average body weights per cage.

Mice each received four oral gavage doses of treatment agents, two on the day before LPS and 2 doses on the morning of LPS treatment at 8 AM and 11 AM. 6 mg/kg LPS (Salmonella typhimurium endotoxin) was administered by intraperitoneal injection at 1 PM. The following morning, 18 hours after LPS injection, rectal body temperatures were measured with a digital thermocouple thermometer. Mice were then anesthetized with isoflurane and blood samples were collected into serum separator tubes. Following coagulation and centrifugation, the resulting serum was analyzed for serum chemistry markers for liver and kidney injury. The treatment schedule is shown in Table 26 below.

TABLE 26
Schedule of GO, Vehicle, GO + Arginine, GO + Citrulline,
and Lysine Orotate Treatments

| | Day 1 | | Day 2 | | | Day 3 |
|---|---|---|---|---|---|---|
| Group (n = 8/grp) | 8 AM | 1 PM | 8 AM | 11 AM | 1 PM | 7 AM |
| Control (no LPS) | HPMC | HPMC | HPMC | HPMC | Saline | Blood |
| | Vehicle | Vehicle | Vehicle | Vehicle | | Collection |
| 8 Am and | HPMC | HPMC | HPMC | HPMC | LPS | Blood |
| 11 AM LPS + | Vehicle | Vehicle | Vehicle | Vehicle | 6 mg/kg | Collection |
| Vehicle | | | | | | |
| LPS + GO | Go | GO | GO | GO | LPS | Blood |
| 522 mg/kg | | | | | 6 mg/kg | Collection |
| LPS + GO + | GO + | GO + | GO + | GO + | LPS | Blood |
| Arginine | Arginine | Arginine | Arginine | Arginine | 6 mg/kg | Collection |
| LPS + GO + | GO + | GO + | GO + | GO + | LPS | Blood |
| Citrulline | Citrulline | Citrulline | Citrulline | Citrulline | 6 mg/kg | Collection |
| LPS + Lysine | GlcN | GlcN | GlcN | GlcN | LPS | Blood |
| Orotate | | | | | 6 mg/kg | Collection |

GO = D-Glucosamine Orotate
LPS = Lipopolysaccharide; endotoxin from Salmonella typhimurium

### Results and Discussion

Nitric Oxide (NO) is an important physiological mediator of vascular tone, affecting blood pressure and delivery of oxygen and nutrients to tissue. NO is produced by nitric oxide synthase, using an amino group on arginine as the immediate precursor. Citrulline is produced as the product of deamination of arginine during formation of NO. However, circulating citrulline can be converted to arginine, especially in the kidney, by scavenging one equivalent of ammonia. In some conditions, citrulline is better for sustained delivery of arginine to tissues, as oral arginine absorbed into the portal vein is efficiently metabolized in the liver, and arginine produced from citrulline in the kidneys bypasses the liver for systemic distribution. The net effect of arginine or orotate in combination with GO is unknown, including in severe systemic inflammation, where NO could either exacerbate damage by enhancing inflammatory hypotension, or it could provide benefits by preventing vasoconstriction without excessive lowering of blood pressure. A combination of GO with either an equimolar dose of arginine or citrulline was tested versus GO alone in a model of LPS-induced septic shock to determine either deleterious or beneficial effects of either amino acid, using serum markers of tissue damage and body temperature maintenance as endpoints.

The data in Table 27 shows that LPS with oral Vehicle resulted in a substantial decrease in body temperature, a protective response to reduce energy expenditure and oxygen requirements, but which generally does not favor survival and recovery. GO treatment resulted in higher core body temperature at 18 hours after LPS, and both Arginine and especially citrulline enhanced the effect of GO on body temperature. Lysine orotate had a beneficial effect on body temperature smaller than that of GO alone.

TABLE 27
Effects of GO alone or with arginine or
citrulline on body temperatures of mice 18
hours after administration of S. typhimurium endotoxin

| | Core Body Temperature (Degrees F) | | |
|---|---|---|---|
| Group | Mean ± SEM | Median | 75^{th} Percentile |
| Normal (no LPS) | 98.3 ± 0.4 | 98.5 | 99.2 |
| LPS + Vehicle | 76.1 ± 0.5 | 75.9 | 77.5 |
| LPS + GO | 82.9 ± 2.5 | 79.0 | 88.5 |
| LPS + GO + Arginine | 85.4 ± 2.4 | 82.4 | 91.5 |
| LPS + GO + Citrulline | 90.7 ± 3.1 | 92.3 | 98.7 |
| LPS + Lysine Orotate | 75.6 ± 0.3 | 78.8 | 82.8 |

The data in Table 28 shows that LPS treatment resulted in substantial elevation of the liver transaminases AST and ALT and both Total and Direct (conjugated) Bilirubin. GO treatment resulted in attenuation of the elevation of AST and ALT, and also reduced elevation of both Total and Direct Bilirubin. Arginine and Citrulline both enhanced the protective effect of GO, with citrulline more effective for reducing these markers than was arginine.

TABLE 28
Serum chemistry markers 18 hours after administration
of S. typhimurium endotoxin.

| | | | TOTAL | BILI- |
|---|---|---|---|---|
| | | | BILI- | RUBIN |
| | ALT | AST | RUBIN | DIRECT |
| Groups | U/L | U/L | mg/dL | mg/dL |
| Control | 52 ± 8 | 81 ± 6 | 0.2 ± 0.0 | 0.3 ± 0.0 |
| (Normal) | | | | |
| LPS + Vehicle | 1695 ± 52 | 1811 ± 53 | 1.3 ± 0.2 | 1.7 ± 0.2 |
| LPS + GO | 1017 ± 62 | 1083 ± 271 | 1.1 ± 0.3 | 1.3 ± 0.4 |
| LPS + GO + | 803 ± 247 | 882 ± 249 | 0.7 ± 0.2 | 0.9 ± 0.2 |
| Arginine | | | | |
| LPS + GO + | 652 ± 276 | 717 ± 269 | 0.5 ± 0.2 | 0.6 ± 0.2 |
| Citrulline | | | | |
| LPS + Lysine | 861 ± 304 | 1171 ± 325 | 0.7 ± 0.2 | 0.9 ± 0.2 |
| Orotate | | | | |

ALT = Alanine Aminotransferase;
AST = Aspartate Aminotransferase

The data in Table 29 shows that LPS also caused hypoglycemia and elevation of Blood Urea Nitrogen (BUN), a marker for kidney injury. As discussed in Example 9 above, high dosages of orotic acid can accumulate in the kidneys, leading to kidney damage. The data in Table 29 shows that BUN was not further elevated beyond the LPS damage after treatment with GO, indicating that plasma orotate after administration of GO was below a threshold for precipitation in the kidneys. Glucose did not elevate above the LPS damage, while creatinine increased slightly above the LPS damage after administration of GO, but not to a level that could cause further kidney damage. Further, the levels of creatinine, glucose, and BUN in mice treated with GO were comparable to those in mice treated with Orotic acid, GlcN, and Orotic acid+GlcN respectively. These results demonstrated that GO did not cause further kidney damage in mice beyond the damage caused by LPS.

TABLE 29
Creatinine, Glucose, and BUN levels 18 hours after
administration of S. typhimurium endotoxin.

| | Creatinine | GLUCOSE | BUN |
|---|---|---|---|
| Mean | mg/dL | mg/dL | mg/dL |
| Control (Normal) | 0.46 ± 0.03 | 240 ± 15 | 21 ± 1 |
| LPS + Vehicle | 0.65 ± 0.03 | 67 ± 5 | 80 ± 3 |
| LPS + GO | 0.81 ± 0.07 | 54 ± 7 | 80 ± 6 |
| LPS + Oro | 0.72 ± 0.10 | 67 ± 8 | 101 ± 11 |
| LPS + GlcN | 0.62 ± 0.10 | 61 ± 5 | 63 ± 11 |
| LPS + Oro + GlcN | 0.89 ± 0.06 | 64 ± 4 | 100 ± 7 |

BUN = Blood Urea Nitrogen

### Example 13: Effects of 5-Hydroxydecanoic Acid (5-HD) on protection by Glucosamine Orotate against LPS-Induced Hypothermia and Liver Damage

Female Balb/C Mice (8 weeks old, weighing >20 grams) housed in a room held at 72-73 degrees F were randomly divided into treatment groups (n=8 mice/group), though with similar average body weights per cage.

GO was dissolved in 0.7% HPMC for oral dosing; HPMC was also the oral vehicle for mice that did not receive GO. LPS and 5-HD were dissolved in 0.9% saline for intraperitoneal (i.p.) injection. Individual treatment doses were 512 mg/kg p.o. of GO, 6 mg/kg i.p. of LPS, and 5 mg/kg i.p. of 5-HD (one hour before LPS and 10 mg/kg i.p. one hour after LPS).

Mice each received four oral gavage doses of treatment agents, two on the day before LPS and 2 doses on the morning of LPS treatment at 8 AM and 11 AM. 6 mg/kg LPS (Salmonella typhimurium endotoxin) was administered by intraperitoneal injection at 1 PM. Two groups of mice received 5-HD 1 hour before and 1 hour after LPS. On following morning at 7 AM, 18 hours after LPS injection, rectal body temperatures were measured with a digital thermocouple thermometer. Mice were then anesthetized with isoflurane and blood samples were collected into serum separator tubes. Following coagulation and centrifugation, the resulting serum was analyzed for serum chemistry markers for liver and kidney injury. The treatment schedule is shown in Table 30 below.

TABLE 30
Schedule of GO, Vehicle, and 5-Hydroxydecanoic Acid Treatments

| | Day 1 | | Day 2 | | | | | Day 3 |
|---|---|---|---|---|---|---|---|---|
| Group (n = 8/grp) | 8 AM | 1 PM | 8 AM | 11 | 12 PM | 1 PM | 2 PM | 7 AM |
| Control (no LPS) | HPMC | HPMC | HPMC | HPMC | Saline | Saline | Saline | Blood |
| | (Vehicle) | | | | | | | Collection |
| LPS + Vehicle | HPMC | HPMC | HPMC | HPMC | Saline | LPS | Saline | Blood |
| | | | | | | | | Collection |
| LPS + GO | GO | GO | GO | GO | Saline | LPS | Saline | Blood |
| | | | | | | | | Collection |
| LPS + GO + | GO | GO | GO | GO | 5-HD | LPS | 5-HD | Blood |
| 5-HD | | | | | | | | Collection |
| LPS + 5-HD | GO | GO | GO | GO | S-HD | LPS | 5-HD | Blood |
| | | | | | | | | Collection |

GO = D-Glucosamine Orotate
LPS = Lipopolysaccharide; endotoxin from Salmonella typhimurium

### Results and Discussion

Orotic acid is an intermediate in de novo synthesis or uridine nucleotides. The only known biochemical fate for the orotate moiety of GO is conversion to orotidine monophosphate (OMP) and then to Uridine Monophosphate (UMP) by the UMP Synthase enzyme complex. UMP is further phosphorylated to yield UTP (uridine triphosphate) which in turn is a precursor for UDP sugars such as UDP-glucose, RNA, and cytidine nucleotides.

Maintenance of thermogenesis and prevention of late (18 hr after LPS) elevation of circulating KC/GRO are consistent with attenuation of LPS-induced mitochondrial dysfunction by GO. An important regulatory effect of uridine nucleotides, specifically UDP (uridine diphosphate), is activation of the mitochondrial ATP-sensitive potassium channel (mito-KATP).

The mito-KATP is embedded in the mitochondrial inner membrane (IM) and mediates regulated passage of potassium ions across the IM to compensate for reciprocal osmotic swelling and shrinkage of the matrix and intermembrane space (IMS), which occurs when there is a mismatch between proton pumping by the electron transport chain (ETC) and utilization of proton gradients for ATP synthesis from ADP. IMS swelling during matrix shrinkage can disrupt the outer membrane, impair the creatine/phosphocreatine cycle for efficient transfer of energy equivalents throughout the cytosol, and inhibit fuel oxidation, thereby exacerbating reductive stress. Mitochondrial membrane permeabilization can result in leakage of mitochondrial DNA (mtDNA) into the cytosol where it can trigger acute or chronic inflammation via the cGAS-STING pathway, an antiviral system that responds to ectopic double-stranded DNA. The cGAS-STING pathway is known to be activated during severe hepatic inflammation induced by LPS, where it amplifies inflammatory damage via activation of IRF3 (interferon response factor 3) and other inflammatory signaling hubs such as NFkB. LPS is also known to cause partial depletion of intact hepatic mtDNA (but not nuclear DNA), consistent with its leakage from damage mitochondria.

The mito-KATP is a fundamental mediator of cytoprotective effects of ischemic preconditioning (IPC), preventing catastrophic tissue failure during subsequent severe ischemia, e.g. in the heart, after induction of IPC brief episodes of protective controlled ischemia. Mito-KATP activation prevents disruption of mitochondrial architecture and outer membrane permeabilization during either excess energy demand or impaired ETC function.

As an alternative to IPC signaling pathways activated by brief ischemia, Uridine Diphosphate (UDP) in the IMS is a potent activator of the mito-KATP, but UTP is not. The UDP/UTP ratio in the IMS is in equilibrium with the ADP/ATP ratio via nucleoside diphosphate kinase; this ratio reflects cellular energy status, integrating cytosolic demand and capacity for ATP synthesis. When the ADP/ATP ratio declines, UDP concentrations in the IMS rise rapidly (UTP normally exceeds UDP) activating the mito-KATP, thereby maintaining mitochondrial membrane architecture and preventing membrane disruption and energy failure.

Without being bound by a specific mechanism, one effect of GO that could contribute to its protective effect against LPS damage could be elevation of intracellular uridine nucleotides sufficient to increase UTP+UDP concentrations in the mitochondrial IMS, which then increase the sensitivity of the mito-KATP to activation during energy distress.

5-HD (5-hydroxydecanoic acid) is a standard inhibitor of the mitochondrial ATP-sensitive potassium channel. 5-HD is known to block both physiological ischemic preconditioning and the cytoprotective effects of exogenous uridine against both mitochondrial energy failure and LPS-induced inflammatory damage to the lungs and other tissues. The effect of 5-HD injections on efficacy of GO in the LPS model of liver inflammatory damage and hypothermia was tested, with 5-HD given by intraperitoneal injection one hour before and 1 hour after LPS.

The data in Table 31 shows that LPS with oral Vehicle resulted in a substantial decrease in body temperature, which is in part a protective response to reduce energy expenditure and oxygen requirements, but which generally does not ultimately favor survival and recovery. Mitochondrial failure also contributes to hypothermia in this model. GO treatment resulted in higher mean and median core body temperature at 18 hours after LPS, and 5-HD markedly reduced the protective effect of GO.

TABLE 31
Effects of GO alone or with 5-Hydroxydecanoic acid
(5-HD) on body temperatures of mice 18 hours after
administration of S. typhimurium endotoxin

| | Core Body Temperature (Degrees F) | | |
|---|---|---|---|
| Group | Mean ± SEM | Median | 75^{th} Percentile |
| Normal (no LPS) | 98.3 ± 0.4 | 98.5 | 99.0 |
| LPS + Vehicle | 77.5 ± 0.3 | 77.9 | 78.0 |
| LPS + GO | 89.3 ± 3.8 | 95.0 | 98.7 |
| LPS + GO + 5-HD | 82.5 ± 2.4 | 78.8 | 86.9 |
| LPS + 5HD | 77.6 ± 0.2 | 77.6 | 78.3 |

The data in Table 32 shows that GO both protected against LPS-induced hypothermia and strongly reduced elevation of serum markers of liver injury. 5-HD markedly reduced the protective effects of GO against both hypothermia and inflammatory liver injury. This strongly supports the idea that GO prevented mitochondrial failure, likely by boosting intracellular and intramitochondrial uridine nucleotides, which facilitates protective activation of the mito-KATP in the liver. The mito-KATP is a primary mediator of protective effects of ischemic preconditioning in tissues, and as such is a key regulator of one of the most important pathways in tissues for preventing bioenergetic failure during compromised delivery of blood, oxygen or nutrients to tissues.

TABLE 32
Effects of GO and 5-Hydroxydecanoic acid on
serum chemistry markers 18 hours
after administration of S. typhimurium endotoxin

| | | | Bilirubin | Bilirubin |
|---|---|---|---|---|
| | | | Total | Direct |
| | ALT U/L | AST U/L | mg/dL | mg/dL |
| | Hepatocyte Damage | | Liver Function | |
| Normal (no LPS) | 78 ± 28 | 93 ± 24 | 0.2 ± 0.0 | 0.2 ± 0.0 |
| LPS + Vehicle | 1721 ± 46 | 1820 ± 49 | 1.2 ± 0.1 | 1.4 ± 0.1 |
| LPS + GO | 159 ± 56 | 263 ± 62 | 0.3 ± 0.0 | 0.3 ± 0.0 |
| LPS + GO + 5HD | 1359 ± 268 | 1527 ± 283 | 1.0 ± 0.3 | 1.4 ± 0.4 |
| LPS + 5HD | 1529 ± 257 | 1660 ± 276 | 1.3 ± 0.2 | 1.6 ± 0.2 |

ALT = Alanine Aminotransferase;
AST = Aspartate Aminotransferase

Activation of the mito-KATP by GO represents a possible mechanism that can account for both prevention of energy failure (reflected in maintenance of thermogenesis after LPS) and prevention of amplification of liver injury that occurs after LPS administration during release of mtDNA, which can in turn activate cGAS-STING or TLR9 signaling, both of which cause inflammation in response to ectopic double-stranded DNA in the cytosol. These results showed that inhibiting the mitochondrial ATP-sensitive potassium channel blocks the protective effects of glucosamine orotate salt in the LPS model on both body temperature and serum markers of liver injury. This was the first demonstration that orotate in any form activates this channel, and it has implications for treating a range of important disorders.

Release of mtDNA in the liver not only triggers local inflammation, but also in other tissues. Some mtDNA released from hepatic mitochondria is exported into circulation, as is cGAMP (cyclic guanosine-adenosine monophosphate), the first mediator of cGAS-STING activation induced by mtDNA. Circulating mtDNA and/or cGAMP can be free in plasma or packaged in extracellular vesicles and can be taken up into multiple cell types where they can activate cGAS-STING pathway signaling and inflammatory injury. cGAS-STING activation contributes to pathological inflammation in a wide variety of both acute and chronic disorders, such as trauma, infection, sepsis, neuroinflammation, chronic inflammation associated with aging, vascular inflammation, including contribution to atherosclerosis, especially in metabolic disorders including type 2 diabetes and fatty liver, and inflammatory bowel diseases. Thus, these results also demonstrated GO's preventative effects in other diseases and disorders related to cGAS-STING activation besides liver.

### Example 14: Time Course of Effects of Glucosamine Orotate Salt on Hypothermia and Circulating Cytokines Induced by Bacterial Endotoxin in Mice

### Materials and Methods

A cohort of 45 eight-week-old female Balb/C mice, more than 20 grams in weight, was divided into six groups of 7 mice each, with three remainder mice serving as untreated Controls.

Mice received four doses of oral D-Glucosamine orotate (GO) (521.9 mg/kg po; 2 doses on the day before and the day of LPS injection) or Vehicle (0.5% hydroxypropyl methylcellulose; HPMC) followed by LPS (6 mg/kg i.p.) one hour after the last dose of oral GO or Vehicle. Rectal temperatures were measured immediately prior to blood collection at the times after LPS indicated for groups in Table 33.

TABLE 33
GO, Vehicle, and Control Groups

| | | | Blood |
|---|---|---|---|
| | | Compound | Collection |
| | Number | (mg/Kg) or | Time after |
| Group | of Mice | HPMC | LPS |
| GO 1 | 7 | GO | 1 hr |
| HPMC 1 | 7 | HPMC | 1 hr |
| GO 4 | 7 | GO | 4 hr |
| HPMC 4 | 7 | HPMC | 4 hr |
| GO 18 | 7 | GO | 18 hr |
| HPMC 18 | 7 | HPMC | 18 hr |
| Control | 3 | — | No LPS |

The rectal temperature of the animals was measured immediately before blood collection by retroorbital bleeding under isoflurane anesthesia into serum separator tubes. After spontaneous clotting, the tubes were centrifuged at 1064 g (10,000 rpm in a 5417R Eppendorf centrifuge with a F-45-30-11 rotor) for 7 minutes to obtain serum samples, which were frozen on dry ice for storage and transport for analysis.

A time course study was conducted on blood samples 1, 4 and 18 hours after administration of LPS to the mice. Cytokine changes in serum in response to LPS in mice pretreated with GO or with Vehicle were measured. Baseline cytokine concentrations were measured in mice that were not treated with LPS. IFN-γ, IL-1β, IL-2, IL-4, IL-5, IL-6, IL-10, IL-12p70, KC/GRO, and TNF-α were quantified in mouse serum samples using the MSD Mouse V-PLEX Proinflammatory Panel 1 (Catalog: N05048A-1, Lot: Z0048042). Measurements were acquired using the MESO SECTOR S 600 MM instrument, and ECL signals were converted to concentration (pg/mL) using a four-parameter logistic (4PL) standard curve fit.

Livers from the same mice used for collection of blood samples for cytokine measurements were removed following blood sampling and euthanasia and were rapidly frozen in liquid nitrogen for subsequent analysis of phosphoprotein signaling components in the cGAS-STING pathway. Liver lysates were prepared by homogenizing the frozen left lateral lobes in 2 ml of RIPA lysis buffer. Mouse liver tissue was homogenized using gentleMACS M Tubes (Order No. 130-093-236, Lot: 9231100233, Miltenyi Biotec) on a gentleMACS Dissociator (SN: 7155, REF: 130-093-235, TYP: A05442, Miltenyi Biotec) using the Protein_01 program. Tissue was lysed in ice-cold pH 8.0 RIPA buffer containing 20 mM Tris-HCl, 150 mM NaCl, 1 mM EDTA, 1 mM EGTA, 1% NP-40, and 0.5% sodium deoxycholate.

Protease inhibitor cocktail (MSD, R0010930) and phosphatase inhibitor cocktails (MSD, R0010916 and R0010963) were added immediately before lysis. Protein concentration was quantified using the Pierce 660 nm Protein Assay Reagent (Ref. 22660, Lot: ZI3983-7). Lysates were aliquoted and stored at −80 °C until further use.

Jess Simple Western System (S/N: JS-5358, ProteinSimple) with 12-230 kDa Separation Kit (Ref: SM-W004-1, Lot: P531756, Protein Simple) and Anti-rabbit Detection Kit (Ref: DM-001, Lot: P533074, Protein Simple) was used to analyze STING, TBK1, and IRF3, along with their phosphorylated forms. Samples and antibodies were prepared according to the manufacturer's protocol. Lysates generated in RIPA lysis buffer supplemented with protease and phosphatase inhibitors were diluted to the desired protein concentration using 0.1× sample buffer. Samples were mixed with 5× master mix (4:1 lysate to buffer), denatured at 70 °C for 20 min, and stored on ice. The 5× master mix contained 400 mM DTT and detergent.

Primary antibodies shown in Table 34 below were diluted in Antibody Diluent 2, and anti-rabbit secondary antibodies were used as provided. Chemiluminescent substrate (luminol+peroxide) was prepared immediately before use. Samples and reagents were loaded into assay plates, centrifuged at 1,000×g for 5 min, and processed on the Jess Simple Western system using Compass (Simple Western) software. The automated workflow included capillary electrophoresis, protein immobilization, immunoprobing, and chemiluminescent detection. Data were analyzed using Compass software to generate electropherograms and quantitative protein expression values.

TABLE 34
List of Antibodies for Analysis of STING, TBK1, and IRF3

| Analyte | Vendor | Catalog | Antigen |
|---|---|---|---|
| pSTING (Ser365 | Abcam | AB324229 | phospho STING |
| mouse, Ser 366 | | | (S365) |
| human) | | | |
| STING/TMEM173 | Novus | NBP3-18816 | human (215-379) |
| pTBK1 (Ser 172 | Abclonal | AP1026 | syntehtic peptide |
| human and mouse) | | | (S172) |
| TBK1 | Novus | NBP3-19537- | human TBK1 |
| | | 100ul | |
| pIRF3 | Thermofisher | PA5-36775 | human IRF3 |
| | | | (phospho 385) |
| IRF3 | Cell Signaling | 4302S | not provided |

### Results and Discussion

### Body Temperature

The data in Table 35 shows that mice treated with intraperitoneal LPS plus oral vehicle displayed severe hypothermia at 18 hours after LPS, but not at the earlier time points, with average body temperature nearly 20 degrees F lower than normal baseline temperature.

TABLE 35
Core Body Temperatures of Mice
Treated with GO vs. Vehicle

| | Core Body Temperature after | | |
|---|---|---|---|
| | LPS (Degrees F, Mean ± SEM) | | |
| | 1 Hour | 4 Hours | 18 Hours |
| Normal (no LPS) | 97.7 ± 0.6 | 97.7 ± 0.6 | 97.7 ± 0.6 |
| LPS + Vehicle | 98.3 ± 0.2 | 97.7 ± 0.3 | 78.1 ± 0.2 |
| LPS + GO | 98.4 ± 0.2 | 99.4 ± 0.3 | 93.1 ± 2.4 |

Survival after LPS

The data in Table 36 shows at the 18 hour time point, 2 mice were found dead in their cages in the LPS+Vehicle group, and one more mouse from this group died after measurement of body temperature but before a blood sample could be collected. No other mice from either group at any other time points died prior to terminal blood sampling.

TABLE 36
Mouse Survival after Treatment with GO vs. Vehicle

| | Mouse Survival After LPS | | |
|---|---|---|---|
| | 1 Hour | 4 Hours | 18 Hours |
| LPS + GO | 7/7 | 7/7 | 7/7 |
| LPS + Vehicle | 7/7 | 7/7 | 4/7 |

Circulating Cytokines in Serum Collected after LPS

After acute administration of bacterial endotoxin (LPS or Lipopolysaccharide) to mammals, several waves of circulating cytokines are produced by several different cell types and with differing durations and times of peak concentrations. Initially, after intraperitoneal injection of LPS, cytokines including TNFa are rapidly released from Kupffer cells and other resident leukocytes in hepatic sinuses. Secondary waves of cytokines reflect tissue responses to the first wave, and include both counter-inflammatory and pro-inflammatory cytokines from various classes of monocytes and DAMPS (disease-associated molecular patterns; endogenous proteins that trigger inflammatory injury). At later time points, persistent or rising serum or plasma cytokines can indicate pathological tissue damage that may not recover rapidly once cytokine waves recede.

Cytokine patterns can provide insight into modes of action of drugs, including agents that protect against inflammatory damage. The data in Tables 37-44 below are grouped by the timing of peak concentrations of cytokines, which reflect sequential processes in the evolution and resolution of inflammatory pathology.

The data in Tables 37 and 38 show concentrations of TNFa and IL-10 at 1 hour, 4 hours, and 18 hours after administration of LPS. The concentrations for TNFa and IL-10 peaked at 1 hour after LPS administration. TNFa is rapidly produced by Kupffer cells, resident macrophages in the hepatic sinuses, in response to LPS; it is considered the critical mediator of LPS-initiated liver injury. The data in Tables 37 and 38 shows that GO did not strongly suppress TNF-α or IL-10 at the 1 and 4 hour time points. The protective effect of GO against severe hypothermia, mortality and liver injury (detected with serum transaminases and bilirubin measurements) was therefore likely not mediated at the level of initial cytokine release from Kupffer cells or other liver-resident monocytes.

TABLE 37
Serum TNF-α of Mice Treated with GO vs.
Vehicle at 1 Hour, 4 Hours, and 18 Hours

| | TNFa after LPS pg/ml (Mean ± SEM) | | |
|---|---|---|---|
| | 1 Hour | 4 Hours | 18 Hours |
| Normal (no LPS) | 0.0 ± 0.0 | 0.0 ± 0.0 | 0.0 ± 0.0 |
| LPS + Vehicle | 10796.6 ± 696.5 | 1393.1 ± 72.1 | 736.9 ± 182.7 |
| LPS + GO | 9011.6 ± 1085.5 | 1228.8 ± 78.7 | 300.5 ± 82.2 |

TABLE 38
Serum IL-10 of Mice Treated with GO vs.
Vehicle at 1 Hour, 4 Hours, and 18 Hours

| | IL-10 after LPS pg/ml (Mean ± SEM) | | |
|---|---|---|---|
| | 1 Hour | 4 Hours | 18 Hours |
| Normal (no LPS) | 10.3 ± 0.8 | 10.3 ± 0.8 | 10.3 ± 0.8 |
| LPS + Vehicle | 1845.7 ± 135.3 | 948.4 ± 48.5 | 1038.8 ± 218.3 |
| LPS + GO | 1893.0 ± 229.6 | 740.6 ± 78.3 | 723.8 ± 0.8 |

The data in Tables 39-42 show concentrations of IL-6, IL-12p70 (IL-12), IL-1b, and IL-5 at 1 hour, 4 hours, and 18 hours after administration of LPS. The concentrations for IL-6, IL-12p70 (IL-12), IL-1b, and IL-5 peaked at 4 hours after LPS administration. The data in Table 39 shows that IL-6 was undetectable in plasma from normal mice not treated with LPS. After LPS, IL-6 was strongly increased, with a peak at 4 hours. GO-treated mice displayed a small reduction in peak IL-6 at 4 hours, but at 18 hours, IL-6 concentrations in the GO-treated group were less than 15% of the concentration in mice pretreated with HPMC vehicle before LPS. The data in Table 40 shows that IL-12 was higher at 18 hours in the LPS+Vehicle group than in the LPS+GO mice. IL-12 is a driver of Interferon-gamma production and may contribute to the elevation of IFN-g at 18 hours in Table 43. Interferon-gamma is produced by several cell types in the liver, especially NK (natural killer) cells.

TABLE 39
Serum IL-6 of Mice Treated with GO vs.
Vehicle at 1 Hour, 4 Hours, and 18 Hours

| | IL-6 after LPS pg/ml (Mean ± SEM) | | |
|---|---|---|---|
| | 1 Hour | 4 Hours | 18 Hours |
| Normal (no LPS) | 0.0 ± 0.0 | 0.0 ± 0.0 | 0.0 ± 0.0 |
| LPS + Vehicle | 32368 ± 1813 | 18177532 ± 1774912 | 402498 ± 138504 |
| LPS + GO | 56035 ± 9228 | 16419418 ± 1739728 | 50128 ± 37877 |

TABLE 40
Serum IL-12p70 of Mice Treated with GO vs.
Vehicle at 1 Hour, 4 Hours, and 18 Hours

| | IL-12p70 after LPS pg/ml (Mean ± SEM) | | |
|---|---|---|---|
| | 1 Hour | 4 Hours | 18 Hours |
| Normal (no LPS) | 0.0 ± 0.0 | 0.0 ± 0.0 | 0.0 ± 0.0 |
| LPS + Vehicle | 393.6 ± 42.0 | 1678.6 ± 111.4 | 822.4 ± 166.5 |
| LPS + GO | 574.6 ± 77.8 | 1459.0 ± 102.0 | 336.8 ± 136.8 |

TABLE 41
Serum IL-1 b of Mice Treated with GO vs.
Vehicle at 1 Hour, 4 Hours, and 18 Hours

| | IL-1b after LPS pg/ml (Mean ± SEM) | | |
|---|---|---|---|
| | 1 Hour | 4 Hours | 18 Hours |
| Normal (no LPS) | 1.1 ± 0.5 | 1.1 ± 0.5 | 1.1 ± 0.5 |
| LPS + Vehicle | 5.3 ± 0.5 | 222.5 ± 18.5 | 131.2 ± 76.1 |
| LPS + GO | 6.1 ± 2.4 | 281.7 ± 30.2 | 23.7 ± 5.8 |

TABLE 42
Serum IL-5 of Mice Treated with GO vs.
Vehicle at 1 Hour, 4 Hours, and 18 Hours

| | IL-5 after LPS pg/ml (Mean ± SEM) | | |
|---|---|---|---|
| | 1 Hour | 4 Hours | 18 Hours |
| Normal (no LPS) | 3.4 ± 0.5 | 3.4 ± 0.5 | 3.4 ± 0.5 |
| LPS + Vehicle | 6.5 ± 0.9 | 43.8 ± 13.1 | 10.8 ± 3.7 |
| LPS + GO | 7.9 ± 0.9 | 73.2 ± 7.8 | 17.4 ± 5.2 |

The data in Tables 43 and 44 show concentrations of IFN-g and KC/GRO at 1 hour, 4 hours, and 18 hours after administration of LPS. The concentrations for IFN-g and KC/GRO peaked at 18 hours after LPS administration. The data in Table 44 shows that GO treatment did not reduce the peak KC/GRO concentration at 4 hours after LPS. In the mice receiving HPMC Vehicle plus LPS, KC/GRO was still elevated at 18 hours, but was strongly diminished at this time point in the mice that received GO treatment. This indicated better maintenance or recovery of metabolic and mitochondrial function in GO-treated mice at the 18 hour time point.

TABLE 43
Serum Interferon-gamma of Mice Treated with
GO vs. Vehicle at 1 Hour, 4 Hours, and 18 Hours

| | Interferon-Gamma after | | |
|---|---|---|---|
| | LPS pg/ml (Mean ± SEM) | | |
| | 1 Hour | 4 Hours | 18 Hours |
| Normal (no LPS) | 1.1 ± 0.2 | 1.1 ± 0.2 | 1.1 ± 0.2 |
| LPS + Vehicle | 1.5 ± 0.1 | 398.4 ± 194.0 | 2183.5 ± 560 |
| LPS + GO | 1.2 ± 0.1 | 277.5 ± 48.9 | 669.8 ± 363.0 |

TABLE 44
Serum KC/GRO of Mice Treated with GO vs.
Vehicle at 1 Hour, 4 Hours, and 18 Hours

| | KC/GRO (CXCL1) after | | |
|---|---|---|---|
| | LPS pg/ml (Mean ± SEM) | | |
| | 1 Hour | 4 Hours | 18 Hours |
| Normal (no LPS) | 72.2 ± 8.4 | 72.2 ± 8.4 | 72.2 ± 8.4 |
| LPS + Vehicle | 54059 ± 2170 | 173007 ± 5212 | 167055 ± 48853 |
| LPS + GO | 60306 ± 5277 | 173322 ± 9694 | 27435 ± 12983 |

KC/GRO is a chemoattractant molecule also known as CXCL1. It acts to recruit monocytes, especially neutrophils, into tissues, in this case the liver, where the infiltrating cells can exacerbate inflammatory injury both by secreting cytokines and by producing Neutrophil Extracellular Traps (NET). Some neutrophils undergo a form of programmed cell death termed “NETosis” in which they extrude their DNA, laden with citrullinated histones (a critical mediator of NETosis is the enzyme PAD4, peptidyl arginine deiminase 4, which converts arginine residues in histones and other proteins to citrulline, resulting in uncoiling and extrusion of neutrophil DNA) and antimicrobial proteins including myeloperoxidase (MPO) and NE (neutrophil elastase (NE). The extruded neutrophil DNA and its associated proteins can trap and inactivate circulating pathogens but also exacerbate inflammatory tissue damage. The human homolog of KC/GRO is IL-8. Human livers secrete IL-8 during surgical stress or trauma; IL-8 is reported to be a better marker for detecting and quantifying liver distress after hepatic surgery than is CRP or other cytokines or acute phase proteins, and more specifically, IL-8 is released from the liver in response to altered NAD+/NADH ratios secondary to mitochondrial and metabolic dysfunction.

The data in Tables 45 and 46 show that IL-2 and IL-4 were not strongly affected by GO. IL-2 is largely produced by CD4+ and CD8+T helper cells and IL-4 is produced by activated Th2 cells. These cell types do not appear to play a major role in LPS-induced liver injury.

TABLE 45
Serum IL-2 of Mice Treated with GO vs.
Vehicle at 1 Hour, 4 Hours, and 18 Hours

| | IL-2 after LPS pg/ml (Mean ± SEM) | | |
|---|---|---|---|
| | 1 Hour | 4 Hours | 18 Hours |
| Normal (no LPS) | 1.9 ± 0.4 | 1.9 ± 0.4 | 1.9 ± 0.4 |
| LPS + Vehicle | 19.1 ± 1.6 | 20.1 ± 1.9 | 4.4 ± 1.1 |
| LPS + GO | 17.9 ± 3.1 | 17.4 ± 1.6 | 15.3 ± 5.2 |

TABLE 46
Serum IL-4 of Mice Treated with GO vs.
Vehicle at 1 Hour, 4 Hours, and 18 Hours

| | IL-4 after LP pg/ml S (Mean ± SEM) | | |
|---|---|---|---|
| | 1 Hour | 4 Hours | 18 Hours |
| Normal (no LPS) | 0.0 ± 0.0 | 0.0 ± 0.0 | 0.0 ± 0.0 |
| LPS + Vehicle | 1.4 ± 0.2 | 1.5 ± 0.1 | 0.5 ± 0.2 |
| LPS + GO | 0.9 ± 0.1 | 1.0 ± 0.2 | 0.0 ± 0.0 |

These results showed that glucosamine orotate salt improves the resilience of hepatocytes in the face of a severe circulating cytokine storm, which is consistent with the mechanism of glucosamine orotate salt preventing mitochondrial failure by activation of the mito-KATP in hepatocytes. The overall pattern of cytokines over time after LPS shows that GO had a small effect on early cytokine release and the underlying cytokine dynamics among monocytes. GO appears to primarily improve resilience of the liver in the presence of inflammatory cytokines that otherwise cause severe liver injury, severe hypothermia, and mortality. GO can be thus complementary to treatments for inflammatory injury that act via modulation of cytokine release from monocytes.

Effects of GO on Intracellular cGAS-STING Pathway Activation by LPS

Consequences of elevated extracellular cytokines are often mediated by intracellular phosphoprotein signaling cascades, which can be assessed by measurement of regulated protein phosphorylation by specific kinases. An important pathway mediating feed-forward amplification of inflammatory injury in the liver and other tissues is the cGAS-STING system, which responds to ectopic double-stranded DNA in the cytosol. cGAS (cyclic Guanosine-Adenosine Monophosphate Synthase) is a cytosolic protein that responds to cytosolic double-stranded DNA by activating interferon signaling and other inflammatory processes. Viral DNA thereby activates an antiviral response, but non-infectious displaced mitochondrial or nuclear DNA can also induce pathological acute or chronic cGAS-STING activation. When cGAS encounters cytosolic double-stranded DNA, it catalyzes formation of cGAMP (cyclic Guanosine-Adenosine Monophosphate), a cyclic dinucleotide. cGAMP activates STING (STimulator of INterferon Genes) resulting in phosphorylation to yield phosphor-STING, which in turn activates IRF3 (Interferon Response Factor 3) with TBK1 (TANK-binding kinase) as an intermediate, also activated by phosphorylation, as well as other intracellular mediators of inflammatory responses including NFkB.

Protein phosphorylation in intracellular signaling pathways can be quantified via Western Blots, in which proteins in a tissue lysate are separated according to molecular weight and net charge by electrophoresis, detected with labeled antibodies and quantified by densitometry of images of electrophoresis gel lanes. Generally, the key parameter is the ratio of phosphorylated to total protein for a specific signaling protein. The Jess system (ProteinSimple, San Jose, CA, USA) acts via similar principles, separating proteins by capillary electrophoresis, facilitating selective binding of appropriate antibodies to selected proteins and quantitation of relative amounts by electropherogram imaging of fluorescence signals from labeled antibodies bound to their target proteins.

This system was used to assess activation of several members of the intracellular cGAS-STING activation pathway in mice treated with LPS, after dosing with oral Glucosamine Orotate or oral Vehicle (HPMC).

The data in Table 47 shows that the ratio of phospho-STING to total STING was higher in the LPS+Vehicle group than in the LS+GO group at both 1 hour and 4 hours after LPS. Similarly, the data in Table 48 shows that at the 4 hour time point, the ratio of phospho-TBK1 to total TBK1 was higher in the LPS+Vehicle group than in animals receiving LPS and GO. These results are consistent with GO reducing activation of the cGAS-STING pathway, with TBK1 downstream of phospho-STING. It is known that mice with genetic downregulation or knockout of cGAS-STING signaling display attenuation of liver damage caused by LPS, and similarly, chemical inhibitors of CGAS-STING pathway activation can also reduce inflammatory injury caused by LPS.

TABLE 47
Effects of GO on Phospho-STING/STING
ratios in livers at 1 and 4 hours after LPS

| | Time | | Phospho-STING/STING Ratios | |
|---|---|---|---|---|
| Group | Point | | Peak Heights | Peak Areas |
| LPS + Vehicle | 1 | hour | 0.155 ± 0.015 | 0.283 ± 0.048 |
| LPS + GO | 1 | hour | 0.092 ± 0.012 * | 0.135 ± 0.061 * |
| | | | * Less than 1 hr LPS ± Vehicle group, | |
| | | | P = 0 .013 | |
| | | | ** Less than 1 hr LPS ± Vehicle group, | |
| | | | P = 0.09 | |
| | | | (Mann Whitney test) | |
| LPS + Vehicle | 4 | hours | 0.332 ± 0.016 | 0.800 ± 0.049 |
| LPS + GO | 4 | hours | 0.153 ± 0.022 | 0.347 ± 0.080 |

* Less than 4 hr LPS ± Vehicle group,
P = 0.008
(Mann Whitney Test)

TABLE 48
Effects of GO on Phospho-TBK1/TBK1
ratios in livers at 4 hours after LPS

| | Time | Phospho-TBK1/TBK1 Ratios | |
|---|---|---|---|
| Group | Point | Peak Heights | Peak Areas |
| LPS + Vehicle | 4 hours | 0.105 ± 0.013 | 0.107 ± 0.010 |
| LPS + GO | 4 hours | 0.055 ± 0.008 | 0.055 ± 0.006 |

* Less than 4 hr LPS ± Vehicle group,
P = 0.03
** Less than 4 hr LPS ± Vehicle group,
P = 0.008
(Mann Whitney Test)

### Example 15: Glucosamine Orotate Stability Study

### Materials and Methods

Three separate batches of D-Glucosamine orotate (GO) were analyzed by HPLC, with UV absorbance detection at multiple wavelengths, and then placed in an incubator at elevated temperature 45 degrees C for 14 days. The high temperature is intended to accelerate and exacerbate potential degradation during storage.

50-100 mg portions of Glucosamine orotate salts were placed onto glass petri dishes without lids and incubated for 2 weeks in digital bench-top incubator with low humidity (<10%).

The control group of samples was kept in tightly closed screw cap glass scintillation vials and in a plastic (HDPE) container.

After the completion of 2 weeks incubation, the exact weights were recorded and compounds were dissolved in water at 1 mM concentrations prior HPLC analysis. A properly calibrated analytical balance and calibrated pipets were used in the experiment, and solutions were prepared in the same protocol for each group and for every lot.

The HPLC analysis included reverse-phase mode chromatography on 1.8-micron particles columns, and absorbance records at 260 nm, 280 nm, 284 nm and 202 nm. The gradient and data acquisition programs were shown to be able to resolve orotate, as well as uracil, uridine, adenosine, AMP, UMP and related compounds, including HMF.

### Results and Discussion

Production of Glucosamine Orotate via ion exchange chromatography, which involves conversion of glucosamine hydrochloride to glucosamine free base, yields glucosamine orotate as an isolatable salt by evaporation of the solvent, which includes water with or without an additional polar solvent such as methanol or ethanol. Aqueous solvent is removed from GO by evaporation under vacuum, optionally in the presence of a desiccant such as phosphorus Pentoxide (P₂O₅). Crystals of GO retain water, which may involve incorporation of water molecules into the crystal lattice, yielding stoichiometric ratios of water to GO. GO produced under different drying conditions can yield crystals with different morphologies and particle sizes, and also with different amounts of incorporated or residual water. For example, GO crystals with 4 molecules of water per molecule of glucosamine orotate were produced by holding GO in a vacuum desiccator with phosphorus pentoxide (P₂O₅) desiccant for 7 days. GO crystals with different morphology and lower water content were produced by holding them under vacuum with P₂O₅ for at least 7 additional days.

The most vulnerable facet of GO to potential chemical degradation during storage is conversion of glucosamine to 5-(hydroxymethyl) furfural (HMF) by elimination of a water molecule. Other reducing sugars can also yield HMF as a degradant. Some glucosamine salts such as glucosamine sulfate are unstable and degrade to HMF and other products over time, limiting their usefulness as dietary supplements or pharmaceutical agents. Relatively stabilized complexes of glucosamine sulfate combined with sodium chloride were reported and used in clinical trials, but were later found to be mixtures of separate crystals of glucosamine hydrochloride and sodium sulfate, both formed after addition of sodium chloride to glucosamine sulfate.

Under the HPLC conditions used for analysis, no impurities, including HMF were detectable. The lower limits of detection (LLOD) for HMF were determined to be 0.052 micromolar, with a lower limit of quantitation of HMF of 0.197 micromolar. The GO and HMF, both standards and samples, were analyzed under the same HPLC conditions, so the maximum amount of HMF that could be present but unseen as a fraction of GO would be 0.052/1000 micromolar=0.000052, or 0.0052%. Therefore, if the minimum amount of detectable HMF was present, the GO would still be 99.9948% pure.

Daily temperatures and humidity were monitored in an incubation chamber. Temperatures remained within a band of 45.3 to 45.7 degrees C, and relative humidity was below 10% on all days as well. After 14 days at 45 degrees C, GO displayed both physical and chemical stability.

Microscopic examination of powders and crystals under 100×, 400× and 1000× magnifications revealed no changes in the shapes, sizes, morphology, and grouping of GO in any of the three lots before and after incubation for 2 weeks at 45 °C and low humidity=<10%.

HPLC analysis of 1 mM solutions of GO lots after incubation showed that no impurities, no new or unknown peaks appeared as a result of 2 weeks incubation. The peak shapes and Retention Times remained the same.

All 3 lots showed reduction in water content as the result of prolonged drying under vacuum with P₂O₅. For example, prior to incubation, lots “GO Lot 002-16-before”, “GO Lot 002-26-before” and “GO Lot 002-32-before” showed corresponding water contents 2.0, 2.4 and 2.3, respectively. After incubation, the water contents decreased to 1.4, 2.1 and 2.1, respectively.

These results demonstrated consistent production of GO with purity essentially 100%, with any impurities below levels of detection under the rigorous HPLC conditions used for analysis. GO was stable under accelerated degradation conditions for 14 days, with no chemical degradation or physical changes in crystalline particles detected, apart from loss of some residual water.

1-63. (canceled)

64. A glucosamine orotate salt.

65. The glucosamine orotate salt of claim 64, having a purity of at least 80%, at least 85%, at least 90%, at least 95%, at least 98%, or at least 99%; or in a range from 80% to 100%, from 85% to 100%, from 90% to 100%, from 95% to 100%, from 98% to 100%, or from 99% to 100%.

66. The glucosamine orotate salt of claim 64, having a purity of at least 99.8%.

67. The glucosamine orotate salt of claim 64, wherein the molar ratio of glucosamine cation and orotate anion in the glucosamine orotate salt is about 1:1.

68. The glucosamine orotate salt of claim 64, in a crystal form.

69. The glucosamine orotate salt of claim 68, wherein the crystal form is a tetrahydrate crystal form, a dihydrate crystal form, and/or a 0.8 hydrate crystal form.

70. The glucosamine orotate salt of claim 68, wherein the crystal form is stable for at least 1 month, at least 3 months, at least 6 months, at least 1 year, at least 2 years, or at least 3 years, at standard pressure, at a temperature ranging from about 3 °C to about 45 °C, and at a humidity of ≤10% or ranging from about 30% to about 60%.

71. A glucosamine orotate salt produced by a process comprising:
(i) passing glucosamine hydrochloride through a column and into a suspension of orotic acid to form the glucosamine orotate salt,
wherein the column comprises an ion exchange resin capable of removing chloride ion from glucosamine hydrochloride to produce glucosamine free base, and
optionally wherein the suspension of orotate acid is under stirring during step (i).

72. A composition comprising
the glucosamine orotate salt of claim 64; and
optionally one or more excipients and/or one or more carriers.

73. The composition of claim 72, comprising one or more excipients and/or one or more carriers.

74. The composition of claim 72, wherein the glucosamine orotate salt is present in an effective amount to improve or maintain the health of the endothelial glycocalyx; and/or
mitochondrial function of a subject.

75. The composition of claim 72, wherein glucosamine orotate salt is present in an effective amount to promote synthesis of glycosaminoglycans and hyaluronan; and/or reduce local and/or systemic inflammation in a subject.

76. The composition of claim 72, wherein the glucosamine orotate salt is present in an effective amount to restore, improve, or support the health of blood vessels, and/or to improve maintenance or repair of cartilage in joints or tendons of a subject.

77. The composition of claim 72, wherein the glucosamine orotate salt is present in an effective amount to restore, improve, or support the health of liver of a subject.

78. The composition of claim 72, wherein the glucosamine orotate salt is present in an effective amount to support glycosaminoglycans and hyaluronan synthesis locally in the skin of a subject.

79. The composition of claim 72, wherein the glucosamine orotate salt is present in an effective amount to reduce and/or ameliorate one or more symptom(s) associated with a mitochondrial disease or disorder of a subject.

80. The composition of claim 72, wherein the glucosamine orotate salt is present in an effective amount to reduce and/or ameliorate one or more symptom(s) associated with a disease or disorder that is associated with cGAS-STING activation of a subject.

81. The composition of claim 72, further comprising one or more citrus flavonoids; and/or one or more nitric oxide elevating compounds.

82. The composition of claim 72, further comprising one or more citrus flavonoids, wherein the one or more citrus flavonoids comprise diosmin and/or hesperidin.

83. The composition of claim 72, further comprising one or more nitric oxide elevating compounds, wherein the one or more nitric oxide elevating compounds comprise arginine, citrulline, nitrate, plant extract(s) with high nitrate content, isosorbide mononitrate, isosorbide dinitrate, nitroglycerin, nicorandil, molsidomine, and/or phosphodiesterase 5 (PDE5) inhibitor(s).

84. The composition of claim 72, in a liquid form or solid form.

85. The composition of claim 72, in the form of dry powder, a tablet, a soft or hard chew, a gummy, a pill, or a capsule.

86. The composition of claim 72, wherein the glucosamine orotate salt is in a crystal form.

87. The composition of claim 72, in a dosage form for administration to a subject, wherein the glucosamine orotate salt is present in an amount ranging from about 300 mg to about 8 g, from about 300 mg to about 4 g, from about 300 mg to about 1 g, from about 300 mg to about 500 mg, from about 1 g to about 8 g, from about 1 g to about 4 g, from about 2 g to about 8 g, or from about 4 g to about 6 g.

88. A method for producing glucosamine orotate salt, comprising:
(i) passing glucosamine hydrochloride through a column and into a suspension of orotic acid to form the glucosamine orotate salt,
wherein the column comprises an ion exchange resin capable of removing chloride ion from glucosamine hydrochloride to produce glucosamine free base, and
optionally wherein the suspension of orotate acid is under stirring during step (i).

89. The method of claim 88, wherein the molar ratio of glucosamine hydrochloride to orotic acid in step (i) is about 1:1.

90. The method of claim 88, wherein the process further comprises, after step (i), (f) isolating the glucosamine orotate salt, optionally step (f) is performed by evaporation of the deionized water and organic solvent under vacuum and optionally at a temperature from about 25 °C to about 50 °C.

91. The method of claim 90, wherein the glucosamine orotate salt is in a crystal form.

92. The method of claim 91, wherein the crystal form is in a tetrahydrate crystal form, a dihydrate crystal form, and/or a 0.8 hydrate crystal form.

93. A method for providing a health benefit to a subject in need thereof comprising:
(i) administering the composition of claim 72 to the subject,
wherein the health benefit is:
(a) improving or maintaining the health of the endothelial glycocalyx;
(b) supporting the health of the blood vessels and/or supporting the synthesis and/or maintenance of cartilage in joints and tendons;
(c) mitigating pain and/or joint damage due to osteoarthritis;
(d) supporting the health of the liver;
(e) protecting the liver against inflammatory damage and/or reducing inflammation in the liver;
(f) improving or maintaining mitochondrial function;
(g) reducing or ameliorating one or more symptoms associated with a mitochondrial disease or disorder; and/or
(h) reducing or ameliorating one or more symptom(s) associated with a disease or disorder that is associated with cGAS-STING activation,
in the subject.
